(19) United States
(12) Patent Application Publication
(10) Pub. No.: US 2024/0317801 A1
(43) Pub. Date: Sep. 26, 2024

(54) PEPTIDOMIMETIC MATRIPTASE 2 INHIBITORS AND USES THEREOF

(71) Applicant: Disc Medicine, Inc., Watertown, MA (US)

(72) Inventors: Srikanth Venkatraman, Edison, NJ (US); Vu Hong, Cambridge, MA (US); Savina Malancona, Rome (IT); Christian Montalbetti, Rome (IT); Paola Fezzardi, Rome (IT)

(73) Assignee: Disc Medicine, Inc., Watertown, MA (US)

(21) Appl. No.: 18/268,107

(22) PCT Filed: Dec. 16, 2021
(86) PCT No.: PCT/US2021/063770

§ 371 Date: Jun. 16, 2023

Related U.S. Application Data
(60) Provisional application No. 63/127,008, filed on Dec. 17, 2020.

(51) Int. Cl. C07K 5/087; A61K 38/00

(52) U.S. Cl. CPC C07K 5/0812; A61K 38/00

(57) ABSTRACT

The present disclosure is directed, in part, to compounds, or pharmaceutically acceptable salts thereof, for modulating the activity of Matriptase 2 (“Mat-2”), or a mutant thereof. The present disclosure also provides pharmaceutically acceptable compositions comprising compounds of the present disclosure and methods of using said compositions in the treatment of various disease and disorders related to Mat-2.

### CROSS REFERENCE TO RELATED APPLICATIONS

This application claims priority to U.S. Provisional Application No. 63/127,008, filed Dec. 17, 2020 which is hereby incorporated by reference in its entirety.

### TECHNICAL FIELD OF THE DISCLOSURE

The present disclosure is directed, in part, to compounds, or pharmaceutically acceptable salts thereof, for modulating the activity of Matriptase 2 (“Mat-2”), or a mutant thereof. The disclosure also provides pharmaceutically acceptable compositions comprising compounds of the present disclosure and methods of using said compositions in the treatment of various diseases and disorders related to Mat-2.

### BACKGROUND OF THE DISCLOSURE

Matriptase-2 is a cell surface serine protease with a modular structure. The enzyme activity of matriptase-2 reduces hepcidin expression through the suppression of bone morphogenetic protein (BMP)/sons of mothers against decapentaplegic homologue protein (SMAD) signaling. Hepcidin regulates the absorption of iron into the body. When the hepcidin level is abnormally low, iron overload occurs. For example, excessive iron absorption is one of the main features of β-thalassemia and can lead to severe morbidity and mortality. Recent evidence suggests that loss of or inhibition of matriptase-2 activity leads to an increase in hepcidin production by the liver, which results in lowered circulating iron burden. Thus there is a need to identify a Mat-2 modulator for the treatment of these and other conditions. The present embodiments described herein fulfill these needs and others.

### SUMMARY OF EMBODIMENTS

The present disclosure provides compounds, or pharmaceutically acceptable salts thereof, in part, modulate the activity of Matriptase-2. The compounds can have, for example, a formula as described herein. In some embodiments, the compound is selected from a compound described herein. In some embodiments, methods of treating the diseases, disorders, and/or conditions, as described herein, are provided.

In some embodiments, the present disclosure provides compounds having a formula of Formula T, or pharmaceutically acceptable salts thereof:

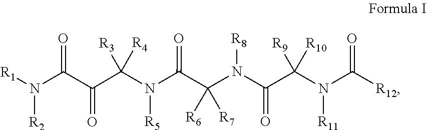

wherein the variables are as defined herein.

The present disclosure also provides pharmaceutical compositions comprising a compound, or a pharmaceutically acceptable salt thereof, as described herein. In some embodiments, the disclosure provides compositions comprising a compound of the present disclosure or a pharmaceutically acceptable derivative thereof and a pharmaceutically acceptable carrier, adjuvant, or vehicle.

The present disclosure also provides methods of using compounds of the present disclosure, pharmaceutically acceptable salts thereof, and pharmaceutically acceptable compositions thereof, for treating a variety of diseases, disorders, or conditions associated with relative or absolute hepcidin deficiency, or diseases, disorders, or conditions in which regulating iron metabolism by increasing hepcidin production by the liver may be therapeutically useful. In some embodiments, the disorder, disease, and/or condition is related to iron overloading. In some embodiments, the iron overload disorder, disease, or condition is hemochromatosis Type 1, 2a, 2b and 3 (hemochromatosis, HFE hemochromatosis (Type 1), juvenile hemochromatosis (types 2a and 2b),), hepcidin deficiency, transfusional iron overload, African iron overload, iron overload cardiomyopathy, or the like.

### DETAILED DESCRIPTION OF CERTAIN EMBODIMENTS

The disclosure may be more fully appreciated by reference to the following description, including the following definitions and examples. Certain features of the disclosed compositions and methods that are provided and described herein in the context of separate aspects may also be provided in combination in a single aspect. Alternatively, various features of the disclosed compositions and methods that are, for brevity, described in the context of a single aspect, may also be provided separately or in any subcombination.

At various places in the present specification, substituents of compounds are disclosed in groups or in ranges. It is specifically intended that the embodiments include each and every individual subcombination of the members of such groups and ranges. For example, the term “C1-6 alkyl” or “C1-C6 alkyl” is specifically intended to individually disclose methyl, ethyl, C3 alkyl, C4 alkyl, C5 alkyl, and C6 alkyl.

It is further appreciated that certain embodiments, which are, for clarity, described in the context of separate embodiments, can also be provided in combination in a single embodiment. Conversely, various features of the embodiments, which are, for brevity, described in the context of a single embodiment, can also be provided separately or in any suitable sub-combination.

All percentages and ratios used herein, unless otherwise indicated, are by weight.

Compounds of the present disclosure include those described generally herein and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “alkyl,” when used alone or as part of a substituent group, refers to a straight- or branched-chain hydrocarbon group, a spirocyclic group, or a fused or bridged bicyclic group, each of which has from 1 to 12 carbon atoms (“C₁-C₁₂”), preferably 1 to 6 carbons atoms (“C₁-C₆”), in the group. Examples of alkyl groups include methyl (Me, C₁ alkyl), ethyl (Et, C₂ alkyl), n-propyl (C₃ alkyl), isopropyl (C₃ alkyl), butyl (C₄ alkyl), isobutyl (C₄ alkyl), sec-butyl (C₄ alkyl), tert-butyl (C₄ alkyl), pentyl (C₅ alkyl), isopentyl (C₅ alkyl), tert-pentyl (C₅ alkyl), hexyl (C₆ alkyl), isohexyl (C₆ alkyl), and the like. The term “spirocyclic group” refers to spirocyclic compounds in which the two rings share only one single atom, the spiro atom, which is usually a quaternary carbon. Examples of spirocyclic compounds are spiro[2,3]undecane, spiro[3,3]heptane, and spiro[5,5]undecane. The term “fused bicyclic group” refers to fused bicyclic compounds, in which two rings share two adjacent atoms. Examples of fused bicyclic compounds include bicyclo[4.4.0]decane, α-thujene and decalin and the like. The term “bridged bicyclic group” refers to bridged bicyclic compounds, in which the two rings share three or more atoms, separating the two bridgehead atoms by a bridge containing at least one atom. Examples of bridged bicyclic compounds include bicyclo[2.2.1]heptane, bicyclo[1,1,1] pentane, bicyclo[2.2.1]heptane, bicyclo[2.2.2]octane, bicyclo[3.3.1]nonane, bicyclo[3.3.3]undecane, and the like. The term “haloalkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group, wherein one or more of the hydrogen atoms in the group have been replaced by a halogen atom. Examples of haloalkyl groups include trifluoromethyl (—CF₃, C₁haloalkyl), trifluoroethyl (—CH₂CF₃, C₂haloalkyl), and the like.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “treatment ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

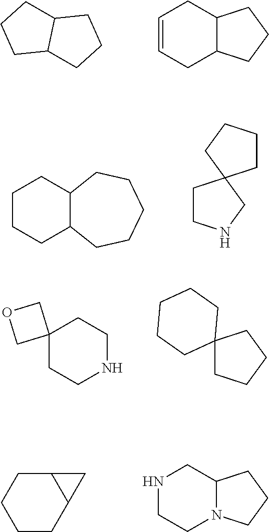

Exemplary bridged bicyclics include:

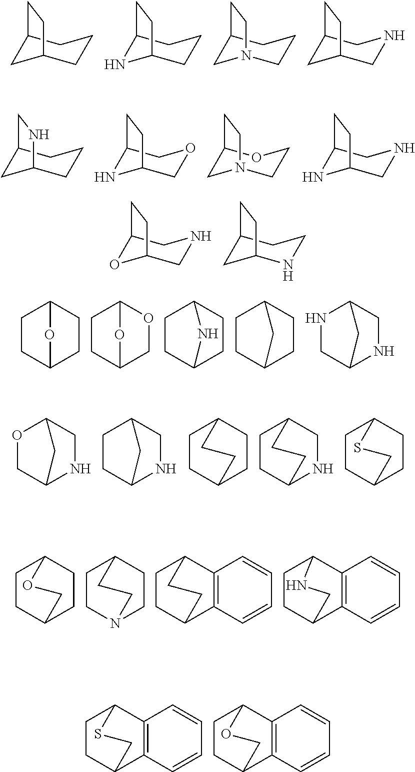

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen or; a substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

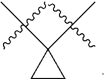

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon, are within the scope of this disclosure. Such compounds are useful, for example, as analytical tools, as probes in biological assays, or as therapeutic agents in accordance with the present disclosure. In certain embodiments, a compound of the disclosure comprises one or more deuterium atoms.

As used herein, the term “inhibitor” is defined as a compound that binds to and/or inhibits Matriptase 2, or a mutant thereof, with measurable affinity. In certain embodiments, an inhibitor has an IC₅₀ and/or binding constant of less than about 100 μM, less than about 50 μM, less than about 20 μM, less than about 10 μM, or less than about 5 μM.

The terms “measurable affinity” and “measurably inhibit,” as used herein, means a measurable change in Matriptase 2, or a mutant thereof, activity between a sample comprising a compound of the present disclosure, or composition thereof, and Matriptase 2, or a mutant thereof, and an equivalent sample comprising Matriptase 2, or a mutant thereof, in the absence of said compound, or composition thereof.

The term “halo” or “halogen” refers to chloro, fluoro, bromo, or iodo.

The term “oxo” refers to an oxygen atom (i.e., ═O) as a divalent substituent, forming a carbonyl group when attached to a carbon (e.g., C═O), or attached to a nitrogen or sulfur heteroatom forming a nitroso, sulfinyl, or sulfonyl.

The term “cycloalkyl” when used alone or as part of a substituent group refers to monocyclic, bicyclic, or tricyclic, non-aromatic hydrocarbon groups having from 3 to 10 carbon atoms (“C₃-C₁₀”), preferably from 3 to 6 carbon atoms (“C₃-C₆”), or from 3 to 7 carbon atoms (“C₃-C₇”). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “heterocycloalkyl” when used alone or as part of a substituent group refers to any three to fourteen membered monocyclic, bicyclic, or tricyclic saturated ring structure containing at least one heteroatom selected from the group consisting of O, N, and S. Heterocycloalkyl groups may be described with respect to the number of atoms in the group, or with respect to the number of carbon atoms in the group.

The term “aryl” when used alone or as part of a substituent group, refers to a mono- or bicyclic-aromatic hydrocarbon ring structure having 6 or 10 carbon atoms in the ring system. Examples of aryl groups are phenyl and naphthyl.

The term “heteroaryl” when used alone or as part of a substituent group, refers to a mono-, bi-, or tricyclic-aromatic ring structure including carbon atoms as well as up to four heteroatoms selected from nitrogen, oxygen, and sulfur. Heteroaryl rings can include a total of 5, 6, 9, 10, or 14 ring atoms. Heteroaryl groups may be described with respect to the number of atoms in the group or with respect to the number of carbon atoms in the group. Thus, the term “5-14 membered heteroaryl” refers to a heteroaryl group containing between 5 and 14 ring atoms. The term —C₄-C₆ heteroaryl, for example, refers to a heteroaryl group containing four to six carbon atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like.

When a range of carbon atoms is used herein, for example, C₁-C₆ all ranges, as well as individual numbers of carbon atoms, are encompassed. For example, “C₁-C₃” includes C₁-C₃, C₁-C₂, C₂-C₃, C₁, C₂, and C₃. The range of carbon atoms may be expressed with alternative expressions. For example, the term “C₁-C₆” is an alternative expression of “C₁-C₆”.

When a ring system is described herein as having a range of members, for example, “5-14-membered”, all ranges, as well as individual numbers of atoms are encompassed. For example, “5-14-membered” includes 5-6-membered, 5-10-membered, 6-9-membered, 5-membered, 6-membered, 7-membered, 8-membered, and the like.

As used herein, “alkoxy” refers to an —O-alkyl group. Example alkoxy groups include methoxy, ethoxy, propoxy (e.g., n-propoxy and isopropoxy), t-butoxy, and the like.

The term “alkenyl” when used alone or as part of a substituent group refers to a straight- or branched-chain group having from 2 to 12 carbon atoms (“C₂-C₁₂”), preferably 2 to 6 carbons atoms (“C₂₋₆”), in the group, wherein the group includes at least one carbon-carbon double bond of alkenyl groups include vinyl (—CH═CH₂; C₂alkenyl), allyl (—CH₂— CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄ alkenyl), 2-butenyl (—CH₂CH═CHCH₃; C₄ alkyl), pentenyl (CH═CHCH₂CH₂CH₃ or CH₂═CHCH₂CH₂CH₂—; C₅ alkenyl), and the like.

The term “alkynyl” when used alone or as part of a substituent group refers to a straight- or branched-chain group having from 2 to 12 carbon atoms (“C₂-C₁₂”), preferably 2 to 6 carbons atoms (“C₂-C₆”), in the group, wherein the group includes at least one carbon-carbon triple bond. Examples of alkynyl groups include ethynyl (—C≡CH; C₂ alkynyl), propargyl (—CH₂—CH≡CH; C₃ alkynyl), and the like.

The compounds described herein can be asymmetric (e.g., having one or more stereocenters). All stereoisomers, such as enantiomers and diastereomers, are intended unless otherwise indicated. Compounds provided herein that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present embodiments. Geometric isomers of the compounds of the present embodiments are described and may be isolated as a mixture of isomers or as separated isomeric forms.

Compounds provided herein may also include tautomeric forms. All tautomeric forms are encompassed.

In some embodiments, the compounds may exist as rotational isomers. In some embodiments, the compounds exist as mixtures of rotational isomers in any proportion. In other embodiments, the compounds exist as particular rotational isomers, substantially free of other rotational isomers.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, and salts thereof, are substantially isolated. By “substantially isolated” is meant that the compound is at least partially or substantially separated from the environment in which is formed or detected. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Also provided herein are pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as, but not limited to, amines; alkali or organic salts of acidic residues such as, but not limited to, carboxylic acids; and the like. The pharmaceutically acceptable salts include, but are not limited to, the conventional non-toxic salts of the parent compound formed, for example, from non-toxic inorganic or organic acids. The pharmaceutically acceptable salts can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound provided herein with one or more solvent molecules.

“Subject” includes humans. The terms “human,” “patient,” and “subject” are used interchangeably herein.

As used herein, the phrase “in need thereof” means that the animal or mammal (subject) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. In some embodiments, the subject in need thereof is suspected of having the condition that needs to be treated.

As used herein, the phrase “integer from X to Y” means any integer that includes the endpoints. For example, the phrase “integer from X to Y” or “1-5” or “1 to 5” means 1, 2, 3, 4, or 5 or any value therein if not modified by the term “integer.”

“Compounds of the present disclosure,” “compounds as described herein” and equivalent expressions, are meant to embrace compounds of any formula or structural representation as described herein, as well as their subgenera, which expression includes the stereoisomers (e.g., enantiomers, diastereomers) and constitutional isomers (e.g., tautomers) of the various compounds and formula provided for herein as well as pharmaceutically acceptable salts or solvates thereof, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of the present disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well known in the art.

Throughout the description, where compositions are described as having, including, or comprising specific components, or where processes are described as having, including, or comprising specific process steps, it is contemplated that compositions described herein also consist essentially of, or consist of, the recited components, and that the processes described herein also consist essentially of, or consist of, the recited processing steps. Further, it should be understood that the order of steps or order for performing certain actions are immaterial so long as the process remains operable. Moreover, two or more steps or actions can be conducted simultaneously.

Compounds of the present disclosure, and pharmaceutical compositions thereof, are useful as inhibitors of Matriptase 2, or a mutant thereof. Without wishing to be bound by any particular theory, it is believed that compounds of the present disclosure, and pharmaceutical compositions thereof, may inhibit the activity of Matriptase 2, or a mutant thereof, and thus treat certain diseases, disorders, or conditions associated with relative or absolute hepcidin deficiency, or diseases, disorders, or conditions in which regulating iron metabolism by increasing hepcidin production by the liver may be therapeutically useful, such as those described herein.

In some embodiments, provided are compounds having a formula of Formula I, or pharmaceutically acceptable salts thereof:

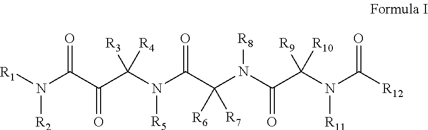

In some embodiments, the disclosure is directed to compounds of Formula (I).

In some embodiments, the disclosure is directed to pharmaceutically acceptable salts or solvates of compounds of Formula (I).

In some embodiments, R₁, R₂, R₃, R₄, R₅, R₆, R₇, R₈, R₉, R₁₀, R₁₁, and R₁₂ are each, independently, are each independently H, D, halo, alkyl, alkoxy, alkenyl, alkynyl, haloalkyl, haloalkoxy, aryl, cycloalkyl, heteroaryl, heterocycloalkyl, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)R^{a}NR^{a}R^{a}, S(O)NR^{a}R^{a}, S(O)₂R^{a}, SF₅, P(O)R^{a}R^{a}, P(O)(OR^{a})(OR^{a}), B(OR^{a})₂ and S(O)₂NR^{a}R^{a}.

In some embodiments, when R₁, R₂, R₃, R₄, R₅, R₆, R₇, R₈, R₉, R₁₀, R₁₁, or R₁₂ is alkyl, alkoxy, alkenyl, alkynyl, haloalkyl, haloalkoxy, aryl, cycloalkyl, heteroaryl, heterocycloalkyl, then R₁, R₂, R₃, R₄, R₅, R₆, R₇, R₈, R₉, R₁₀, R₁₁, or R₁₂ is optionally substituted with at least one R^{b} substituent.

In some embodiments, R₁, R₂, R₃, R₄, R₅, R₆, R₇, R₈, R₉, R₁₀, R₁₁, or R₁₂ is H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a} S(O)₂R^{a}, SF₅, P(O)R^{a}R^{a}, P(O)(OR^{a})(OR^{a}), B(OR^{a})₂, or S(O)₂NR^{a}R^{a}.

In some embodiments, when R₁, R₂, R₃, R₄, R₅, R₆, R₇, R₈, R₉, R₁₀, R₁₁, or R₁₂ is C₁₋₆ alkyl, C₁₋₆ alkoxy, C₂₋₆alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, or (4-14 membered heterocycloalkyl)-C₁₋₄ alkyl, then R₁, R², R³, R⁴, or R⁵ is optionally substituted with 1, 2, 3, 4 or 5 independently selected R^{b} substituents.

In some embodiments, R₁, R₂, R₃, R₄, R₅, R₆, R₇, R₈, R₉, R₁₀, R₁₁, or R₁₂ is C₁₋₆alkyl, for example, C₆ alkyl, C₅ alkyl, C₄ alkyl, C₃ alkyl, C₂ alkyl, C₁ alkyl, methyl, ethyl, isopropyl, and the like, wherein R₁, R₂, R₃, R₄, R₅, R₆, R₇, R₈, R₉, R₁₀, R₁₁, or R₁₂ is optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is C₁₋₆ alkoxy, for example, C₆ alkoxy, C₅ alkoxy, C₄ alkoxy, C₃ alkoxy, C₂ alkoxy, C₁ alkoxy, methoxy, ethoxy, isopropoxy, and the like, wherein R₁, R₂, R₃, R₄, R₅, R₆, R₇, R₈, R₉, R₁₀, R₁₁, or R₁₂ is optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is C₂₋₆ alkenyl, for example, C₆ alkenyl, C₅ alkenyl, C₄ alkenyl, C₃ alkenyl, C₂ alkenyl, ethenyl, propenyl, isopropenyl, and the like, wherein R₁, R₂, R₃, R₄, R₅, R₆, R₇, R₈, R₉, R₁₀, R₁₁, or R₁₂ is optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is C₂₋₆ alkynyl, for example, C₆ alkynyl, C₅ alkynyl, C₄ alkynyl, C₃ alkynyl, C₂ alkynyl, ethynyl, 2-propynyl (i.e., propargyl), and the like, substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is C₆₋₁₀ aryl, for example, C₆ aryl, C₇ aryl, C₈ aryl, C₉ aryl, C₁₀ aryl, phenyl, naphthyl, and the like, optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is C₃₋₁₀cycloalkyl, for example, C₁₀cycloalkyl, C₉cycloalkyl, C₈cycloalkyl, C₇cycloalkyl, C₆ cycloalkyl, C₅ cycloalkyl, C₄ cycloalkyl, C₃ cycloalkyl, cyclopropyl, cyclobutyl, cyclopentyl, cyclohexyl, cycloheptyl and the like, optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is cyclopropyl, optionally substituted with 1, 2, 3, 4, or 5 independently selected R^{b} substituents. In some embodiments, R₁, R₂, R₃, R₄, R₅, R₆, R₇, R₈, R₉, R₁₀, R₁₁, or R₁₂ is cyclobutyl, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is a 5-14 membered heteroaryl, for example, 5 membered heteroaryl, 6 membered heteroaryl, 7 membered heteroaryl, 8 membered heteroaryl, 9 membered heteroaryl, 10 membered heteroaryl, 11 membered heteroaryl, 12 membered heteroaryl, 13 membered heteroaryl, 14 membered heteroaryl, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is 4-10 membered heterocycloalkyl, for example, 10 membered heterocycloalkyl, 9 membered heterocycloalkyl, 8 membered heterocycloalkyl; 7 membered heterocycloalkyl, 6 membered heterocycloalkyl, 5 membered heterocycloalkyl, 4 membered heterocycloalkyl, piperidinyl, pyrrolidinyl, tetrahydropyranyl, tetrahydrofuranyl, oxetanyl, and the like, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is C₆₋₁₀ aryl-C₁₋₄ alkyl, for example, C₆₋₁₀ aryl-C₁ alkyl, C₆₋₁₀ aryl-C₂ alkyl, C₆₋₁₀ aryl-C₃ alkyl, C₆₋₁₀ aryl-C₄ alkyl, C₆ aryl-C₁ alkyl, C₆ aryl-C₂ alkyl, C₆ aryl-C₃ alkyl, C₆ aryl-C₄ alkyl, C₁₀ aryl-C₁ alkyl, C₁₀ aryl-C₂ alkyl, C₁₀ aryl-C₃ alkyl, C₁₀ aryl-C₄ alkyl, —CH₂-phenyl, —CH₂CH₂-phenyl, —CH₂— naphthyl, —CH₂CH₂-naphthyl, and the like, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is C₃₋₁₀ cycloalkyl-C₁₋₄ alkyl, for example, C₃₋₁₀ cycloalkyl-C₁ alkyl, C₃₋₁₀ cycloalkyl-C₂ alkyl, C₃₋₁₀ cycloalkyl-C₃ alkyl, C₃₋₁₀ cycloalkyl-C₄ alkyl, C₃₋₆ cycloalkyl-C₁ alkyl, C₃₋₆ cycloalkyl-C₂ alkyl, C₃₋₆ cycloalkyl-C₃ alkyl, C₃₋₆ cycloalkyl-C₄ alkyl, C₅₋₆ cycloalkyl-C₁ alkyl, C₅₋₆ cycloalkyl-C₂ alkyl, C₅₋₆ cycloalkyl-C₃ alkyl, C₅₋₆ cycloalkyl-C₄ alkyl, and the like, optionally substituted with 1, 2, 3, 4, or 5 independently selected R^{b} substituents.

In some embodiments, R₁, R₂, R₃, R₄, R₅, R₆, R₇, R₈, R₉, R₁₀, R₁₁, or R₁₂ is cyclopropylmethyl, optionally substituted with 1, 2, 3, 4, or 5 independently selected R^{b} substituents. In some embodiments, R₁, R₂, R₃, R₄, R₅, R₆, R₇, R₈, R₉, R₁₀, R₁₁, or R₁₂ is cyclobutylmethyl, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is a 5-14 membered heteroaryl, for example, 5 membered heteroaryl, 6 membered heteroaryl, 7 membered heteroaryl, 8 membered heteroaryl, 9 membered heteroaryl, 10 membered heteroaryl, 11 membered heteroaryl, 12 membered heteroaryl, 13 membered heteroaryl, 14 membered heteroaryl, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like, optionally substituted with 1, 2, 3, 4, or 5 independently selected R^{b} substituents.

In other embodiments, R₁, R₂, R₃, R₄, R₅, R₆, R₇, R₈, R₉, R₁₀, R₁₁, or R₁₂ is 4-10 membered heterocycloalkyl, for example, 10 membered heterocycloalkyl, 9 membered heterocycloalkyl, 8 membered heterocycloalkyl; 7 membered heterocycloalkyl, 6 membered heterocycloalkyl, 5 membered heterocycloalkyl, 4 membered heterocycloalkyl, piperidinyl, pyrrolidinyl, tetrahydropyranyl, tetrahydrofuranyl, and the like, optionally substituted with 1, 2, 3, 4, or 5 independently selected R^{b} substituents.

In other embodiments, or one or more selected from the group consisting of R₁ and R₂, R₁ and R₄, R₃ and R₄, R₆ and R₇, R₇ and R₈, and R₉ and R₁₀, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In other embodiments, or one or more selected from the group consisting of R₁ and R₄, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In other embodiments, or one or more selected from the group consisting of R₃ and R₄, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In other embodiments, or one or more selected from the group consisting of R₆ and R₇, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In other embodiments, or one or more selected from the group consisting of R₇ and R₈, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In other embodiments, or one or more selected from the group consisting of R₉ and R₁₀, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents.

In some embodiments, each R^{a} is independently selected from H, D, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl.

In some embodiments, when R^{a} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a} is optionally substituted with 1, 2, 3, 4, or 5 independently selected R^{d} substituents.

In some embodiments, each R^{b} substituent is independently selected from a bond, H, D, halo, oxo, C₁₋₁₀ alkyl, C₁₋₆ alkoxy, C₁₋₄haloalkyl, C₁₋₄haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR'S(O)R^{c}, NR'S(O)(═NR^{c})R^{c}, NR'S(O)₂R^{c}, NR'S(O)₂NR^{c}R^{c}, S(O)R^{c}, S(O)NR^{c}R^{c}, S(O)₂R^{c} or S(O)₂NR^{c}R^{c};

In some embodiments, when R^{b} is C₁₋₁₀ alkyl, C₁₋₆ alkoxy, C₁₋₄haloalkyl, C₁. 4haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{b} is optionally substituted with 1, 2, or 3 independently selected R^{d} substituents.

In some embodiments, each R^{c} is independently selected from H, D, OH, C₁₋₆ alkyl, C₁₋₆ alkoxy, C₁₋₄haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl.

In some embodiments, when R^{c} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents.

In some embodiments, each R^{f} is independently selected from halogen, C₁₋₁₀ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R_{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R^{g}, P(O)(OR^{g})(OR^{g}), S(O)R^{g}, S(O)NR^{g}R^{g}, S(O)₂R₉, NR^{g}S(O)₂R^{g}, NR^{g} S(O)₂NR^{g}R^{g}, and S(O)₂NR^{g}R^{g};

In some embodiments, when R^{f} is C₁₋₄alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆. 10 aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents.

In some embodiments, each Rⁿ is independently selected from C₁₋₁₀ alkyl, C₁₋₄haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}.

In some embodiments, each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, SF₅, P(O)R^{e}R^{e}, P(O)(OR^{e})(OR^{e}), S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e},

In some embodiments, when R^{d} is C₁₋₆alkyl, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents.

In some embodiments, each R^{e} is independently selected from H, D, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,

In some embodiments, when R^{e} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents.

In some embodiments, each R^{g} is independently selected from H, D, C₁₋₆ alkyl, C₁₋₄haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,

In some embodiments, when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents.

In some embodiments, each R^{P} is independently selected from C₁₋₁₀ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r}.

In some embodiments, each R^{o} or R^{r} is independently selected from H, D, C₁₋₁₀ alkyl, C₃₋₆cycloalkyl, C₆₋₁₀ aryl, 5 or 6-membered heteroaryl, C₁₋₄haloalkyl, C₂₋₄alkenyl, and C₂₋₄ alkynyl,

In some embodiments, when R^{o} or R^{r} is C₁₋₁₀ alkyl, C₃₋₆ cycloalkyl, C₆₋₁₀ aryl, 5 or 6-membered heteroaryl, C₂₋₄ alkenyl, and C₂₋₄ alkynyl, then R^{o} or R^{r} is optionally substituted with 1, 2 or 3 independently selected R^{q} substituents.

In some embodiments, each R^{q} is independently selected from D, OH, CN, —COOH, NH₂, halo, C₁₋₆alkyl, C₁₋₆ haloalkyl, C₁₋₆ alkoxy, C₁₋₆ haloalkoxy, C₁₋₄ alkylthio, phenyl, 5-6 membered heteroaryl, C₃₋₆ cycloalkyl, 4-6 membered heterocycloalkyl, —CONHR^{t}, —NHC(O)R^{t}, —OC(O)R^{t}, —C(O)OR^{t}, —C(O)R^{t}, —SO₂R^{t}, —NHSO₂R^{t}, —SO₂NHR^{t} and NR^{t}R^{t},

In some embodiments, when R^{q} is C₁₋₆ alkyl, phenyl, 4-6 membered heterocycloalkyl or 5-6 membered heteroaryl, then R^{q} is optionally substituted with OH, CN, —COH, NH₂, C₁₋₆ alkoxy, C₃₋₆cycloalkyl or 4-6 membered heterocycloalkyl; and

In some embodiments, each R^{t} is independently C₁₋₆ alkyl.

In some embodiments, R₁, R₂, R₃, R₄, R₅, R₆, R₇, R₈, R₉, R₁₀, R₁₁, and R₁₂ are each, independently, H, D, OH, NH₂, NHC(═NH)NH₂, NHR^{a}, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In other embodiments, R₁, R₂, R₃, R₄, R₅, R₆, R₇, R₈, R₉, R₁₀, R₁₁, and R₁₂ are each, independently, H. In other embodiments, R₁, R₂, R₃, R₄, R₅, R₆, R₇, R₈, R₉, R₁₀, R₁₁, and R₁₂ are each, independently, D. In other embodiments, R₁, R₂, R₃, R₄, R₅, R₆, R₇, R₈, R₉, R₁₀, R₁₁, and R₁₂ are each, independently, OH. In other embodiments, R₁, R₂, R₃, R₄, R₅, R₆, R₇, R₈, R₉, R₁₀, R₁₁, and R₁₂ are each, independently, NH₂. In other embodiments, R₁, R₂, R₃, R₄, R₅, R₆, R₇, R₈, R₉, R₁₀, R₁₁, and R₁₂ are each, independently, NHC(═NH)NH₂. In other embodiments, R₁, R₂, R₃, R₄, R₅, R₆, R₇, R₈, R₉, R₁₀, R₁₁, and R₁₂ are each, independently, NHR^{a}. In other embodiments, R₁, R₂, R₃, R₄, R₅, R₆, R₇, R₈, R₉, R₁₀, R₁₁, and R₁₂ are each, independently, optionally substituted C₁-C₆ alkyl. In other embodiments, R₁, R₂, R₃, R₄, R₅, R₆, R₇, R₈, R₉, R₁₀, R₁₁, and R₁₂ are each, independently, optionally substituted C₁-C₆ hydroxyalkyl. In other embodiments, R₁, R₂, R₃, R₄, R₅, R₆, R₇, R₈, R₉, R₁₀, R₁₁, and R₁₂ are each, independently, optionally substituted C₁-C₆ alkoxy. In other embodiments, R₁, R₂, R₃, R₄, R₅, R₆, R₇, R₈, R₉, R₁₀, R₁₁, and R₁₂ are each, independently, optionally substituted cycloalkyl. In other embodiments, R₁, R₂, R₃, R₄, R₅, R₆, R₇, R₈, R₉, R₁₀, R₁₁, and R₁₂ are each, independently, optionally substituted cycloheteroalkyl.

In some embodiments, R₅ is H.

In some embodiments, R₈ is H.

In some embodiments, R₁₁ is H.

In some embodiments, R₃ is H.

In some embodiments, R₆ is H.

In some embodiments, R₉ is H.

In some embodiments, provided are compounds having a formula of

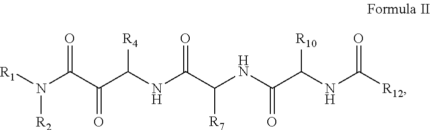

or pharmaceutically acceptable salts or solvates thereof, wherein the variables are as defined herein.

In some embodiments, the disclosure is directed to compounds of Formula (II).

In some embodiments, the disclosure is directed to pharmaceutically acceptable salts or solvates of compounds of Formula (II).

In some embodiments, provided are compounds has a formula of

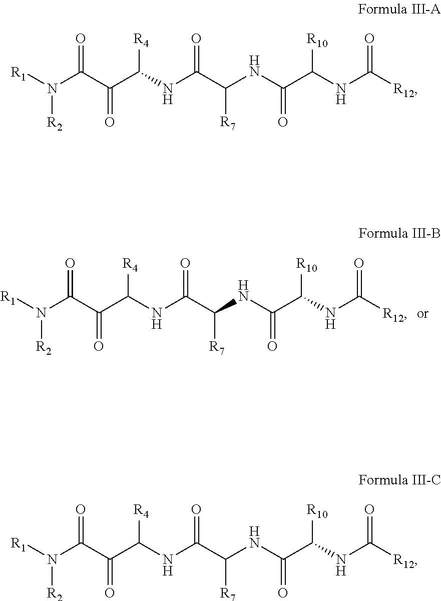

or pharmaceutically acceptable salts or solvates thereof, wherein the variables are as defined herein. In some embodiments, provided are compounds has a formula of

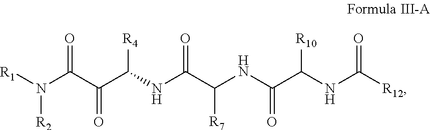

or pharmaceutically acceptable salts or solvates thereof, wherein the variables are as defined herein. In some embodiments, provided are compounds has a formula of

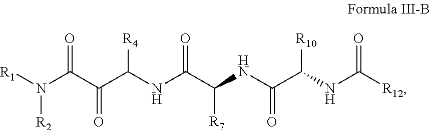

or pharmaceutically acceptable salts or solvates thereof, wherein the variables are as defined herein. In some embodiments, provided are compounds has a formula of

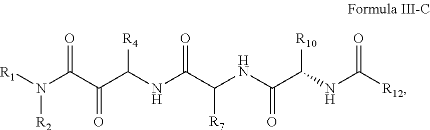

or pharmaceutically acceptable salts or solvates thereof, wherein the variables are as defined herein.

In some embodiments, the disclosure is directed to compounds of Formula (III-A).

In some embodiments, the disclosure is directed to pharmaceutically acceptable salts or solvates of compounds of Formula (III-A).

In some embodiments, the disclosure is directed to compounds of Formula (III-B).

In some embodiments, the disclosure is directed to pharmaceutically acceptable salts or solvates of compounds of Formula (III-B).

In some embodiments, the disclosure is directed to compounds of Formula (III-C).

In some embodiments, the disclosure is directed to pharmaceutically acceptable salts or solvates of compounds of Formula (III-C).

In some embodiments, provided are compounds having a formula of

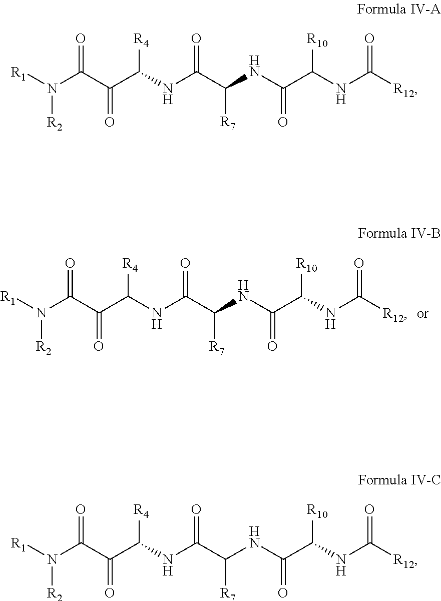

or pharmaceutically acceptable salts or solvates thereof, wherein the variables are as defined herein. In some embodiments, provided are compounds having a formula of

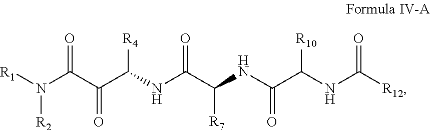

or pharmaceutically acceptable salts or solvates thereof, wherein the variables are as defined in herein. In some embodiments, provided are compounds having a formula of

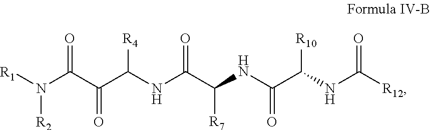

or pharmaceutically acceptable salts or solvates thereof, wherein the variables are as defined in herein. In some embodiments, provided are compounds having a formula of

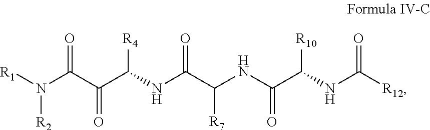

or pharmaceutically acceptable salts or solvates thereof, wherein the variables are as defined herein.

In some embodiments, provided are compounds having a formula of

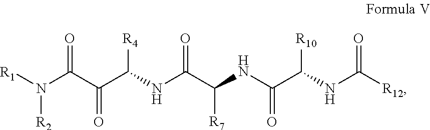

wherein the variables are as defined in herein.

In some embodiments, the disclosure is directed to compounds of Formula (IV-A).

In some embodiments, the disclosure is directed to pharmaceutically acceptable salts or solvates of compounds of Formula (IV-A).

In some embodiments, the disclosure is directed to compounds of Formula (IV-B).

In some embodiments, the disclosure is directed to pharmaceutically acceptable salts or solvates of compounds of Formula (IV-B).

In some embodiments, the disclosure is directed to compounds of Formula (IV-C).

In some embodiments, the disclosure is directed to pharmaceutically acceptable salts or solvates of compounds of Formula (IV-C).

In some embodiments, R₁ is optionally substituted C₁-C₆ alkyl.

In some embodiments, R₁ is Me.

In some embodiments, R₁ is H.

In some embodiments, R₂ is H, Me, Et, t-Bu, —CH₂CN, -3-pyridyl, —CH₂C(O)OR₁₇,

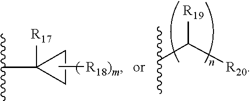

In some embodiments, R₂ is Me. In some embodiments, R₂ is Et. In some embodiments, R₂ is H. In some embodiments, R₂ is t-Bu. In some embodiments, R₂ is —CH₂CN. In some embodiments, R₂ is -3-pyridyl. In some embodiments, R₂ is —CH₂C(O)OR₁₇. In some embodiments, R₂ is

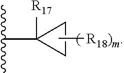

In some embodiments, R₂ is

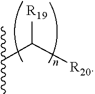

In some embodiments, R₁₇, R₁₈, R₁₉, and R₂₀ are each, independently, H, OH, NH₂, optionally substituted C₁-C₆ alkyl, optionally substituted C₁-C₆ hydroxyalkyl, optionally substituted C₁-C₆ alkoxy, optionally substituted cycloalkyl, optionally substituted pyridyl, optionally substituted heteroaryl, optionally substituted aryl, or optionally substituted cycloheteroalkyl. In some embodiments, R₁₇, R₁₈, R₁₉, and R₂₀ are each, independently, H. In some embodiments, R₁₇, R₁₈, R₁₉, and R₂₀ are each, independently, OH. In some embodiments, R₁₇, R₁₈, R₁₉, and R₂₀ are each, independently, NH₂. In some embodiments, R₁₇, R₁₈, R₁₉, and R₂₀ are each, independently, optionally substituted C₁-C₆ alkyl. In some embodiments, R₁₇, R₁₈, R₁₉, and R₂₀ are each, independently, optionally substituted C₁-C₆ hydroxyalkyl. In some embodiments, R₁₇, R₁₈, R₁₉, and R₂₀ are each, independently, optionally substituted C₁-C₆ alkoxy. In some embodiments, R₁₇, R₁₈, R₁₉, and R₂₀ are each, independently, optionally substituted cycloalkyl. In some embodiments, R₁₇, R₁₈, R₁₉, and R₂₀ are each, independently, optionally substituted pyridyl. In some embodiments, R₁₇, R₁₈, R₁₉, and R₂₀ are each, independently, optionally substituted heteroaryl. In some embodiments, R₁₇, R₁₈, R₁₉, and R₂₀ are each, independently, optionally substituted aryl. In some embodiments, R₁₇, R₁₈, R₁₉, and R₂₀ are each, independently, optionally substituted cycloheteroalkyl.

In some embodiments, m is 0-4. In some embodiments, m is 1-4. In some embodiments, m is 2-4. In some embodiments, m is 0-4. In some embodiments, m is 3-4. In some embodiments, m is 0. In some embodiments, m is 1. In some embodiments, m is 2. In some embodiments, m is 3. In some embodiments, m is 4.

In some embodiments, n is 0-10. In some embodiments, n is 1-10. In some embodiments, n is 2-10. In some embodiments, n is 3-10. In some embodiments, n is 4-10. In some embodiments, n is 5-10. In some embodiments, n is 6-10. In some embodiments, n is 7-10. In some embodiments, n is 8-10. In some embodiments, n is 9-10. In some embodiments, n is 10. In some embodiments, n is 9. In some embodiments, n is 8. In some embodiments, n is 7. In some embodiments, n is 6. In some embodiments, n is 5. In some embodiments, n is 4. In some embodiments, n is 3. In some embodiments, n is 2. In some embodiments, n is 1.

In some embodiments, R₂ is Me.

In some embodiments, R₂ is Et.

In some embodiments, R₂ is 3-pyridyl.

In some embodiments, R₁₇ is Me.

In some embodiments, R₁₈ is halo.

In some embodiments, R₁₈ is F.

In some embodiments, R₁₉ is H.

In some embodiments, R₂₀ is 3-pyridyl, —C(O)OR₂₁, or CN. In some embodiments, R₂₀ is 3-pyridyl. In some embodiments, R₂₀ is —C(O)OR₂₁. In some embodiments, R₂₀ is CN.

In some embodiments, R₂₁. is optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂₁. is optionally substituted C₁-C₆ alkyl. In some embodiments, R₂₁. is optionally substituted C₁-C₆ hydroxyalkyl. In some embodiments, R₂₁. is optionally substituted C₁-C₆ alkoxy. In some embodiments, R₂₁. is optionally substituted cycloalkyl. In some embodiments, R₂₁. is optionally substituted cycloheteroalkyl. In some embodiments, R₂₁ is ethyl.

In some embodiments, R₂ and R₄ together with the atom to which they are attached, form a 3-, 4-, 5-, 6-, or 7-membered cycloalkyl or heterocycloalkyl ring optionally substituted with 1, 2, 3, 4 or 5 independently selected R^{b} substituents, wherein R^{b} is as defined herein. In some embodiments, R₂ and R₄ together with the atom to which they are attached, form a 3-membered cycloalkyl or heterocycloalkyl ring optionally substituted with 1, 2, 3, 4 or 5 independently selected R^{b} substituents, wherein R^{b} is as defined herein. In some embodiments, R₂ and R₄ together with the atom to which they are attached, form a 4-membered cycloalkyl or heterocycloalkyl ring optionally substituted with 1, 2, 3, 4 or 5 independently selected R^{b} substituents, wherein R^{b} is as defined herein. In some embodiments, R₂ and R₄ together with the atom to which they are attached, form a 5-membered cycloalkyl or heterocycloalkyl ring optionally substituted with 1, 2, 3, 4 or 5 independently selected R^{b} substituents, wherein R^{b} is as defined herein. In some embodiments, R₂ and R₄ together with the atom to which they are attached, form a 6-membered cycloalkyl or heterocycloalkyl ring optionally substituted with 1, 2, 3, 4 or 5 independently selected R^{b} substituents, wherein R^{b} is as defined herein. In some embodiments, R₂ and R₄ together with the atom to which they are attached, form a 7-membered cycloalkyl or heterocycloalkyl ring optionally substituted with 1, 2, 3, 4 or 5 independently selected R^{b} substituents, wherein R^{b} is as defined herein.

In some embodiments, R₄ is

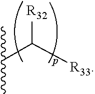

In some embodiments, R₃₂ and R₃₃ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

In some embodiments, p is 0-10. In some embodiments, p is 1-10. In some embodiments, p is 2-10. In some embodiments, p is 3-10. In some embodiments, p is 4-10. In some embodiments, p is 5-10. In some embodiments, p is 6-10. In some embodiments, p is 7-10. In some embodiments, p is 8-10. In some embodiments, p is 9-10. In some embodiments, p is 10. In some embodiments, p is 9. In some embodiments, p is 8. In some embodiments, p is 7. In some embodiments, p is 6. In some embodiments, p is 5. In some embodiments, p is 4. In some embodiments, p is 3. In some embodiments, p is 2. In some embodiments, p is 1.

In some embodiments, R₃₂ is H.

In some embodiments, R₃₃ is H,

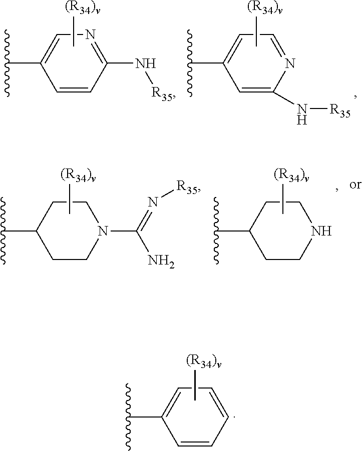

In some embodiments, R₃₃ is H. In some embodiments, R₃₃ is

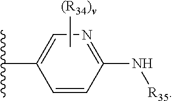

In some embodiments, R₃₃ is

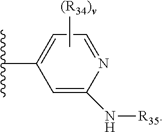

In some embodiments, R₃₃ is

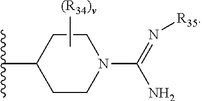

In some embodiments, R₃₃ is

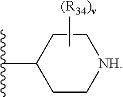

In some embodiments, R₃₃ is

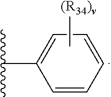

In some embodiments, R₃₄ and R₃₅ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₃₄ and R₃₅ are each, independently, H, In some embodiments, R₃₄ and R₃₅ are each, independently, D. In some embodiments, R₃₄ and R₃₅ are each, independently, optionally substituted C₁-C₆ alkyl. In some embodiments, R₃₄ and R₃₅ are each independently, optionally substituted C₁-C₆ hydroxyalkyl. In some embodiments, R₃₄ and R₃₅ are each, independently, optionally substituted C₁-C₆ alkoxy. In some embodiments, R₃₄ and R₃₅ are each, independently, optionally substituted cycloalkyl. In some embodiments, R₃₄ and R₃₅ are each, independently, optionally substituted cycloheteroalkyl.

In some embodiments, v is 0-5. In some embodiments, v is 0-4. In some embodiments, v is 1-4. In some embodiments, v is 2-4. In some embodiments, v is 0-4. In some embodiments, v is 3-4. In some embodiments, v is 0. In some embodiments, v is 1. In some embodiments, v is 2. In some embodiments, v is 3. In some embodiments, v is 4. In some embodiments, v is 5.

In some embodiments, R₃₄ is H. In some embodiments, v is 0. In some embodiments, v is 1.

In some embodiments, R₃₅ is H.

In some embodiments, R₃₃ is

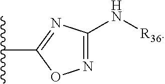

In some embodiments, R₃₆ is H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₃₆ is H. In some embodiments, R₃₆ is D. In some embodiments, R₃₆ is optionally substituted C₁-C₆ alkyl. In some embodiments, R₃₆ is optionally substituted C₁-C₆ hydroxyalkyl. In some embodiments, R₃₆ is optionally substituted C₁-C₆ alkoxy. In some embodiments, R₃₆ is optionally substituted cycloalkyl. In some embodiments, R₃₆ is optionally substituted cycloheteroalkyl. In some embodiments, R₃₆ is H.

In some embodiments, R₃₃ is

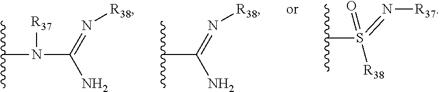

In some embodiments, R₃₃ is

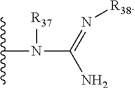

In some embodiments, R₃₃ is

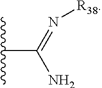

In some embodiments, R₃₃ is

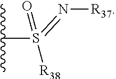

In some embodiments, R₃₇ and R₃₈ are each, independently, H, D, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₃₇ and R₃₈ are each, independently, H. In some embodiments, R₃₇ and R₃₈ are each independently, D. In some embodiments, R₃₇ and R₃₈ are each independently, NH₂. In some embodiments, R₃₇ and R₃₈ are each, independently, optionally substituted C₁-C₆ alkyl. In some embodiments, R₃₇ and R₃₈ are each independently, optionally substituted C₁-C₆ hydroxyalkyl. In some embodiments, R₃₇ and R₃₈ are each, independently, optionally substituted C₁-C₆ alkoxy. In some embodiments, R₃₇ and R₃₈ are each, independently, optionally substituted cycloalkyl. In some embodiments, R₃₇ and R₃₈ are each, independently, optionally substituted cycloheteroalkyl.

In some embodiments, R₃₇ is H.

In some embodiments, R₃₈ is H or NH₂.

In some embodiments, R₃₈ is optionally substituted C₁-C₆ alkyl.

In some embodiments, R₃₈ is Me.

In some embodiments, R₃₈ is optionally substituted C₁-C₆ alkoxy or NH₂.

In some embodiments, R₃₈ is MeO.

In some embodiments, R₇ is

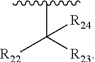

In some embodiments, R₂₂, R₂₃, and R₂₄, are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂₂, R₂₃, and R₂₄ are each, independently, H. In some embodiments, R₂₂, R₂₃, and R₂₄, are each independently, D. In some embodiments, R₂₂, R₂₃, and R₂₄, are each, independently, optionally substituted C₁-C₆ alkyl. In some embodiments, R₂₂, R₂₃, and R₂₄, are each, independently, optionally substituted C₁-C₆ hydroxyalkyl. In some embodiments, R₂₂, R₂₃, and R₂₄, are each independently, optionally substituted C₁-C₆ alkoxy. In some embodiments, R₂₂, R₂₃, and R₂₄ are each, independently, optionally substituted cycloalkyl. In some embodiments, R₂₂, R₂₃, and R₂₄ are each, independently, optionally substituted cycloheteroalkyl.

In some embodiments, R₂₂ and R₂₃ together with the atom to which they are attached, form an optionally substituted cycloalkyl or optionally substituted cycloheteroalkyl. In some embodiments, R₂₂ and R₂₃ together with the atom to which they are attached, form an optionally substituted cycloalkyl. In some embodiments, R₂₂ and R₂₃ together with the atom to which they are attached, form an optionally substituted cycloheteroalkyl.

In some embodiments, R₇ is

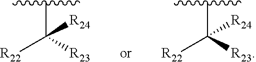

In some embodiments, R₇ is

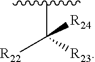

In some embodiments, R₇ is

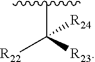

In some embodiments, R₂₂, R₂₃, and R₂₄ are as defined herein.

In some embodiments, R₂₂ is Me.

In some embodiments, R₂₃ is OH, OMe, Me, or Et. In some embodiments, R₂₃ is OH. In some embodiments, R₂₃ is OMe. In some embodiments, R₂₃ is Me. In some embodiments, R₂₃ is Et.

In some embodiments, R₂₂ and R₂₃ together with the atom to which they are attached, form an optionally substituted cyclobutane.

In some embodiments, R₂₂ and R₂₃ together with the atom to which they are attached, form an optionally substituted cyclopetane or cyclohexane. In some embodiments, R₂₂ and R₂₃ together with the atom to which they are attached, form an optionally substituted cyclopetane. In some embodiments, R₂₂ and R₂₃ together with the atom to which they are attached, form an optionally substituted cyclohexane.

In some embodiments, R₂₄ is Me.

In some embodiments, R₂₄ is H.

In some embodiments, R₇ and R₈ are together with the atoms to which they are attached, form a 3-, 4-, 5-, 6-, or 7-membered cycloalkyl or heterocycloalkyl ring optionally substituted with 1, 2, 3, 4 or 5 independently selected R^{b} substituents, wherein R^{b} is as defined herein. In some embodiments, R₇ and R₈ are together with the atoms to which they are attached, form a 3-membered cycloalkyl or heterocycloalkyl ring optionally substituted with 1, 2, 3, 4 or 5 independently selected R^{b} substituents, wherein R^{b} is as defined herein. In some embodiments, R₇ and R₈ are together with the atoms to which they are attached, form a 4-membered cycloalkyl or heterocycloalkyl ring optionally substituted with 1, 2, 3, 4 or 5 independently selected R^{b} substituents, wherein R^{b} is as defined herein. In some embodiments, R₇ and R₈ are together with the atoms to which they are attached, form a 5-membered cycloalkyl or heterocycloalkyl ring optionally substituted with 1, 2, 3, 4 or 5 independently selected R^{b} substituents, wherein R^{b} is as defined herein. In some embodiments, R₇ and R₈ are together with the atoms to which they are attached, form a 6-membered cycloalkyl or heterocycloalkyl ring optionally substituted with 1, 2, 3, 4 or 5 independently selected R^{b} substituents, wherein R^{b} is as defined herein. In some embodiments, R₇ and R₈ are together with the atoms to which they are attached, form a 7-membered cycloalkyl or heterocycloalkyl ring optionally substituted with 1, 2, 3, 4 or 5 independently selected R^{b} substituents, wherein R^{b} is as defined herein.

In some embodiments, R₇ and R₈ together are

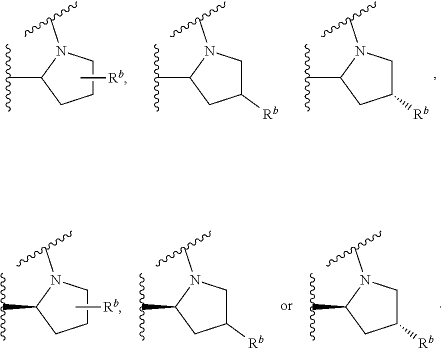

In some embodiments, R₇ and R₈ together are

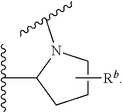

In some embodiments, R₇ and R₈ together are

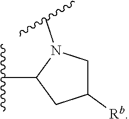

In some embodiments, R₇ and R₈ together are

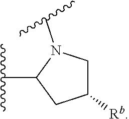

In some embodiments, R₇ and R₈ together are

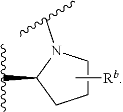

In some embodiments, R₇ and R₈ together are

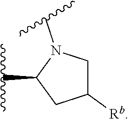

In some embodiments, R₇ and R₈ together are

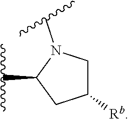

In some embodiments, R^{b} is as defined herein. In some embodiments, R^{b} is H. In some embodiments, R^{b} is OH or CN.

In some embodiments, R₁₀ is

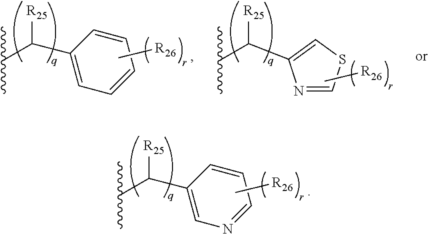

In some embodiments, R₁₀ is

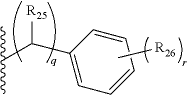

In some embodiments, R₁₀ is

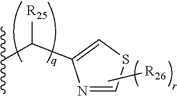

In some embodiments, R₁₀ is

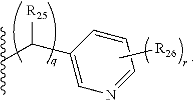

In some embodiments, R₂₅ and R₂₆ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl. In some embodiments, R₂₅ and R₂₆ are each, independently, H. In some embodiments, R₂₅ and R₂₆, are each, independently, D. In some embodiments, R₂₅ and R₂₆ are each independently, optionally substituted C₁-C₆ alkyl. In some embodiments, R₂₅ and R₂₆ are each independently, optionally substituted C₁-C₆ hydroxyalkyl. In some embodiments, R₂₅ and R₂₆ are each independently, optionally substituted C₁-C₆ alkoxy. In some embodiments, R₂₅ and R₂₆ are each independently, optionally substituted cycloalkyl. In some embodiments, R₂₅ and R₂₆ are each independently, optionally substituted cycloheteroalkyl.

In some embodiments, q is 0-10. In some embodiments, q is 1-10. In some embodiments, q is 2-10. In some embodiments, q is 3-10. In some embodiments, q is 4-10. In some embodiments, q is 5-10. In some embodiments, q is 6-10. In some embodiments, q is 7-10. In some embodiments, q is 8-10. In some embodiments, q is 9-10. In some embodiments, q is 10. In some embodiments, q is 9. In some embodiments, q is 8. In some embodiments, q is 7. In some embodiments, q is 6. In some embodiments, q is 5. In some embodiments, q is 4. In some embodiments, q is 3. In some embodiments, q is 2. In some embodiments, q is 1.

In some embodiments, r is 0-5. In some embodiments, r is 0-4. In some embodiments, r is 1-4. In some embodiments, r is 2-4. In some embodiments, r is 0-4. In some embodiments, r is 3-4. In some embodiments, r is 0. In some embodiments, r is 1. In some embodiments, r is 2. In some embodiments, r is 3. In some embodiments, r is 4. In some embodiments, r is 5.

In some embodiments, R₂₅ is H. In some embodiments, q is 1. In some embodiments, q is 2, 3, or 4. In some embodiments, r is 0. In some embodiments, r is 1.

In some embodiments, R₂₆ is Me.

In some embodiments, R₂₆ is halo.

In some embodiments, R₂₆ is F.

In some embodiments, R₁₂ is

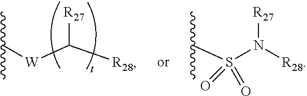

In some embodiments, R₁₂ is

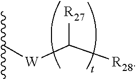

In some embodiments, R₁₂ is or

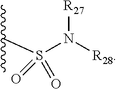

In some embodiments, W is O, S, NR₂₉, S(O)₂NR₂₉, S(O)₂, S(O)₂NH, or CR₃₀R₃₁. In some embodiments, W is O. In some embodiments, W is S. In some embodiments, W is NR₂₉. In some embodiments, W is S(O)₂NR₂₉. In some embodiments, W is S(O)₂. In some embodiments, W is S(O)₂NH. In some embodiments, W is CR₃₀R₃₁.

In some embodiments, R₂₇, R₂₈, R₂₉, R₃₀ and R₃₁ are each, independently, H, D, optionally substituted C₁-C₆ alkyl, optionally substituted C₁-C₆ hydroxyalkyl, optionally substituted C₁-C₆ alkoxy, optionally substituted cycloalkyl, optionally substituted aryl, optionally substituted heteroaryl, or optionally substituted cycloheteroalkyl. In some embodiments, R₂₇, R₂₈, R₂₉, R₃₀ and R₃₁ are each, independently, H. In some embodiments, R₂₇, R₂₈, R₂₉, R₃₀ and R₃₁ are each independently, D. In some embodiments, R₂₇, R₂₈, R₂₉, R₃₀ and R₃₁ are each, independently, optionally substituted C₁-C₆ alkyl. In some embodiments, R₂₇, R₂₈, R₂₉, R₃₀ and R₃₁ are each independently, optionally substituted C₁-C₆ hydroxyalkyl. In some embodiments, R₂₇, R₂₈, R₂₉, R₃₀ and R₃₁ are each independently, optionally substituted C₁-C₆ alkoxy. In some embodiments, R₂₇, R₂₈, R₂₉, R₃₀ and R₃₁ are each, independently, optionally substituted cycloalkyl. In some embodiments, R₂₇, R₂₈, R₂₉, R₃₀ and R₃₁ are each, independently, optionally substituted aryl. In some embodiments, R₂₇, R₂₈, R₂₉, R₃₀ and R₃₁ are each, independently, optionally substituted heteroaryl. In some embodiments, R₂₇, R₂₈, R₂₉, R₃₀ and R₃₁ are each, independently, optionally substituted cycloheteroalkyl.

In some embodiments, t is 0-10. In some embodiments, t is 1-10. In some embodiments, t is 2-10. In some embodiments, t is 3-10. In some embodiments, t is 4-10. In some embodiments, t is 5-10. In some embodiments, t is 6-10. In some embodiments, t is 7-10. In some embodiments, t is 8-10. In some embodiments, t is 9-10. In some embodiments, t is 10. In some embodiments, t is 9. In some embodiments, t is 8. In some embodiments, t is 7. In some embodiments, t is 6. In some embodiments, t is 5. In some embodiments, t is 4. In some embodiments, t is 3. In some embodiments, t is 2. In some embodiments, t is 1.

In some embodiments, W is CH₂.

In some embodiments, W is NR₂₉,

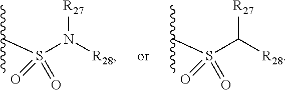

In some embodiments, W is NR₂₉,

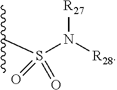

In some embodiments, W is NR₂₉,

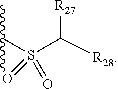

In some embodiments, R₂₉ is H.

In some embodiments, R₂₇ is H.

In some embodiments, t is 1-4.

In some embodiments, R₂₈ is methyl, ethyl, cyclopropyl, or phenyl. In some embodiments, R₂₈ is methyl. In some embodiments, R₂₈ is ethyl. In some embodiments, R₂₈ is cyclopropyl. In some embodiments, R₂₈ is phenyl.

In some embodiments, R₂₈ is optionally substituted C₁-C₆ alkyl.

In some embodiments, R₂₈ is Me

In some embodiments, R₂₈ is Et.

In some embodiments, R₂₈ is isopropyl.

In some embodiments, provided is any compound as described herein.

In some embodiments, provided is a compound, or a pharmaceutically acceptable salt, having a formula of

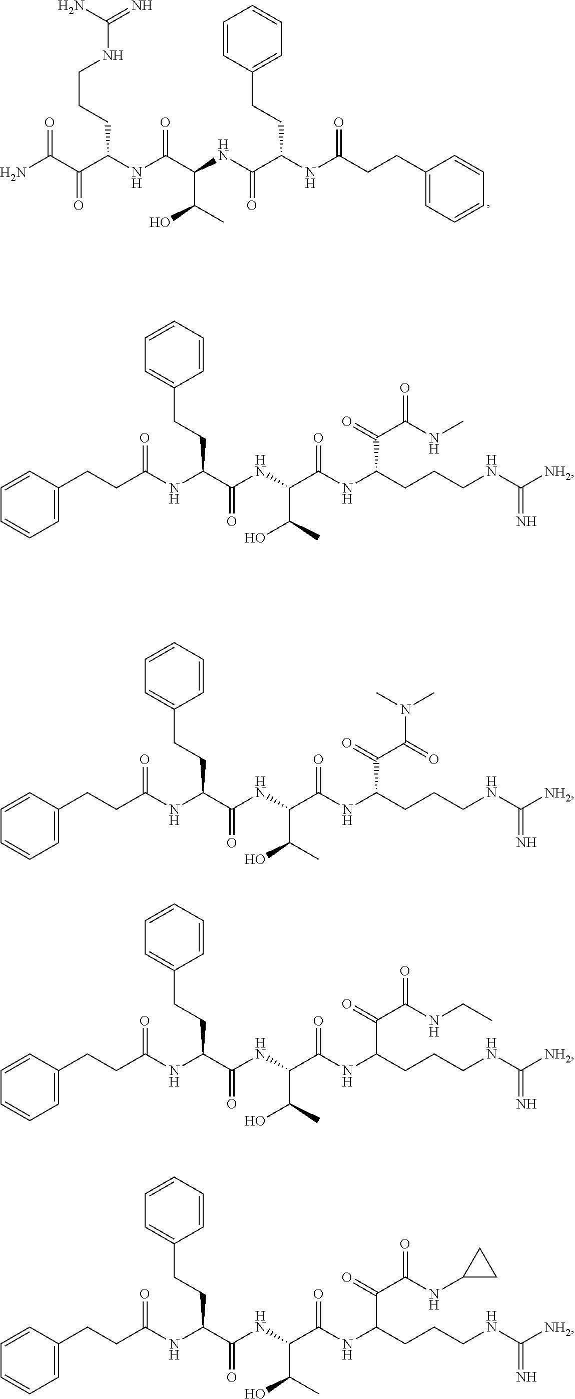 [SMILES: C[C@@H](O)[C@H](NC(=O)[C@H](CCc1ccccc1)NC(=O)CCc1ccccc1)C(=O)N[C@@H](CCCNC(=N)N)C(=O)C(N)=O ; "," ; CNC(=O)C(=O)[C@H](CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)N[C@@H](CCCNC(=N)N)C(=O)C(=O)N(C)C ; CCNC(=O)C(=O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NC1CC1]

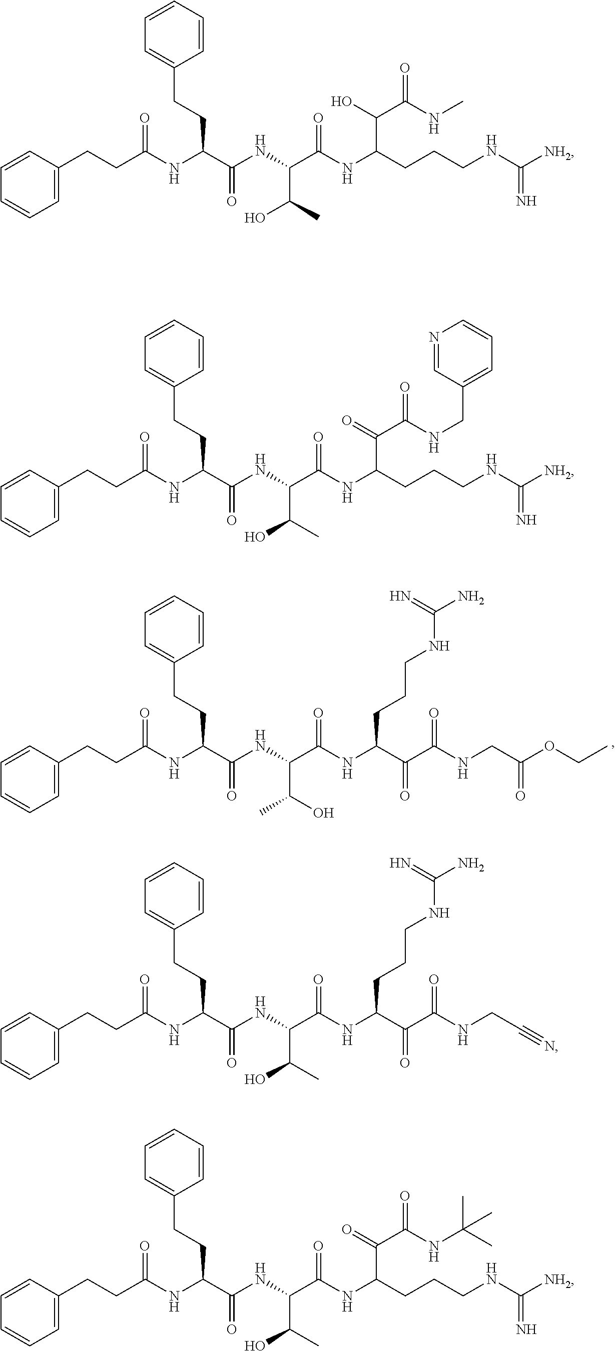 [SMILES: CNC(=O)C(O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NCc1cccnc1 ; CCOC(=O)CNC(=O)C(=O)[C@H](CCCNC(=N)N)NC(=O)[C@@H](NC(=O)[C@H](CCc1ccccc1)NC(=O)CCc1ccccc1)[C@@H](C)O ; "," ; C[C@@H](O)[C@H](NC(=O)[C@H](CCc1ccccc1)NC(=O)CCc1ccccc1)C(=O)N[C@@H](CCCNC(=N)N)C(=O)C(=O)NCC#N ; C[C@@H](O)[C@H](NC(=O)[C@H](CCc1ccccc1)NC(=O)CCc1ccccc1)C(=O)NC(CCCNC(=N)N)C(=O)C(=O)NC(C)(C)C]

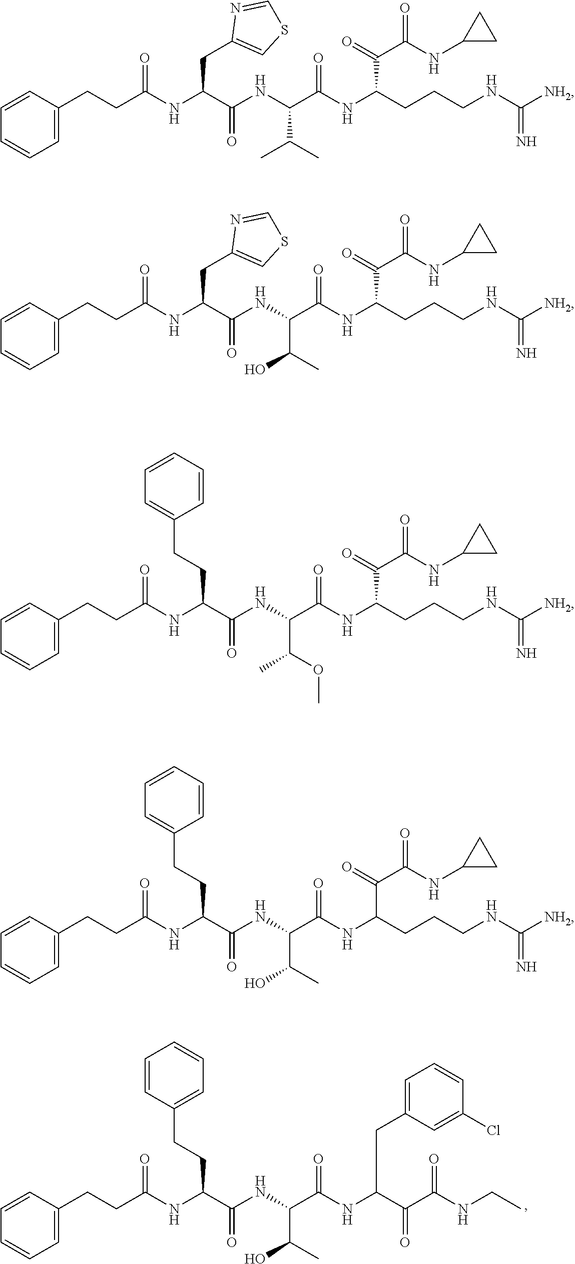 [SMILES: CC(C)[C@H](NC(=O)[C@H](Cc1cscn1)NC(=O)CCc1ccccc1)C(=O)N[C@@H](CCCNC(=N)N)C(=O)C(=O)NC1CC1 ; C[C@@H](O)[C@H](NC(=O)[C@H](Cc1cscn1)NC(=O)CCc1ccccc1)C(=O)N[C@@H](CCCNC(=N)N)C(=O)C(=O)NC1CC1 ; CO[C@H](C)[C@H](NC(=O)[C@H](CCc1ccccc1)NC(=O)CCc1ccccc1)C(=O)N[C@@H](CCCNC(=N)N)C(=O)C(=O)NC1CC1 ; C[C@H](O)[C@H](NC(=O)[C@H](CCc1ccccc1)NC(=O)CCc1ccccc1)C(=O)NC(CCCNC(=N)N)C(=O)C(=O)NC1CC1 ; CCNC(=O)C(=O)C(Cc1cccc(Cl)c1)NC(=O)[C@@H](NC(=O)[C@H](CCc1ccccc1)NC(=O)CCc1ccccc1)[C@@H](C)O ; ","]

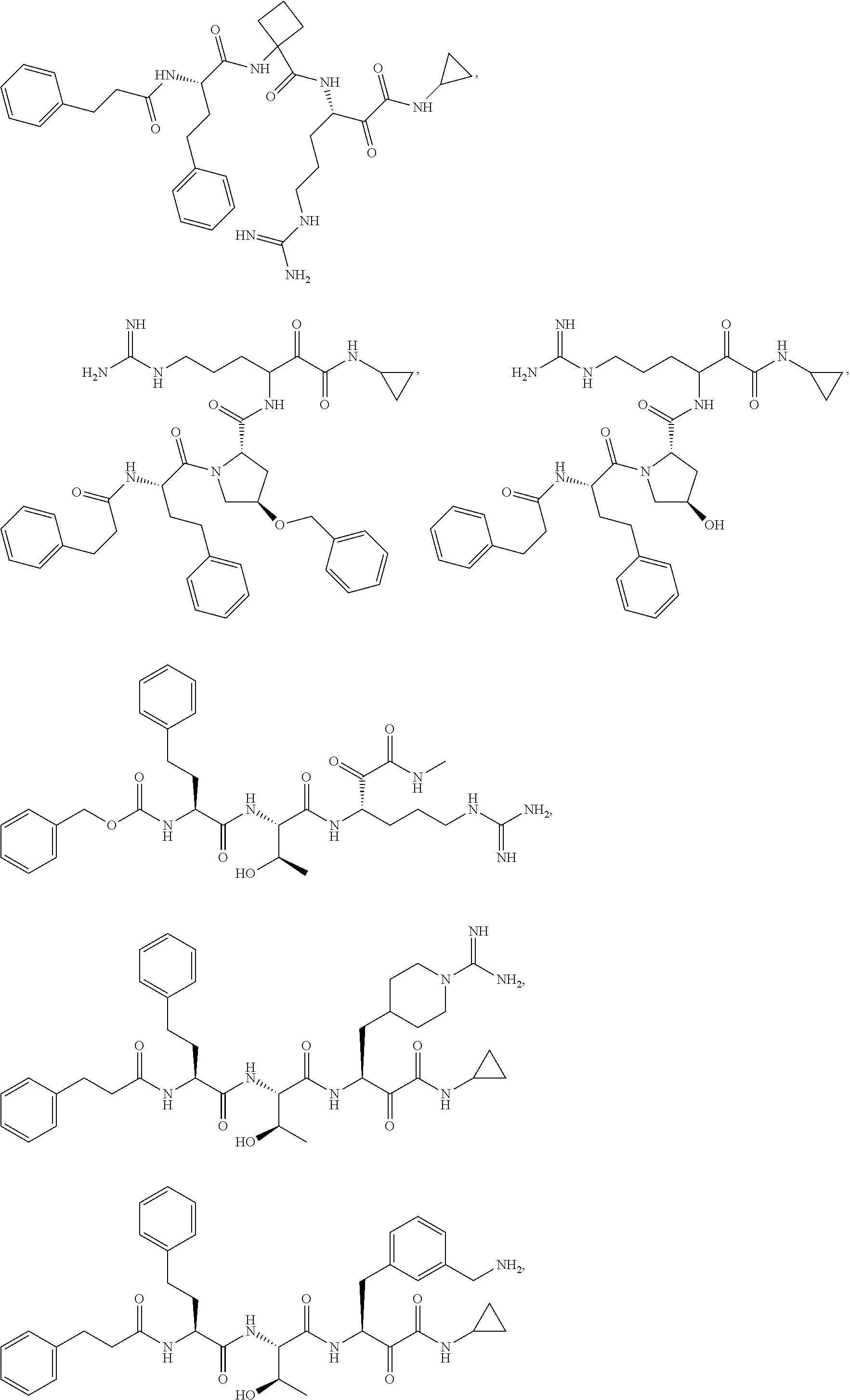

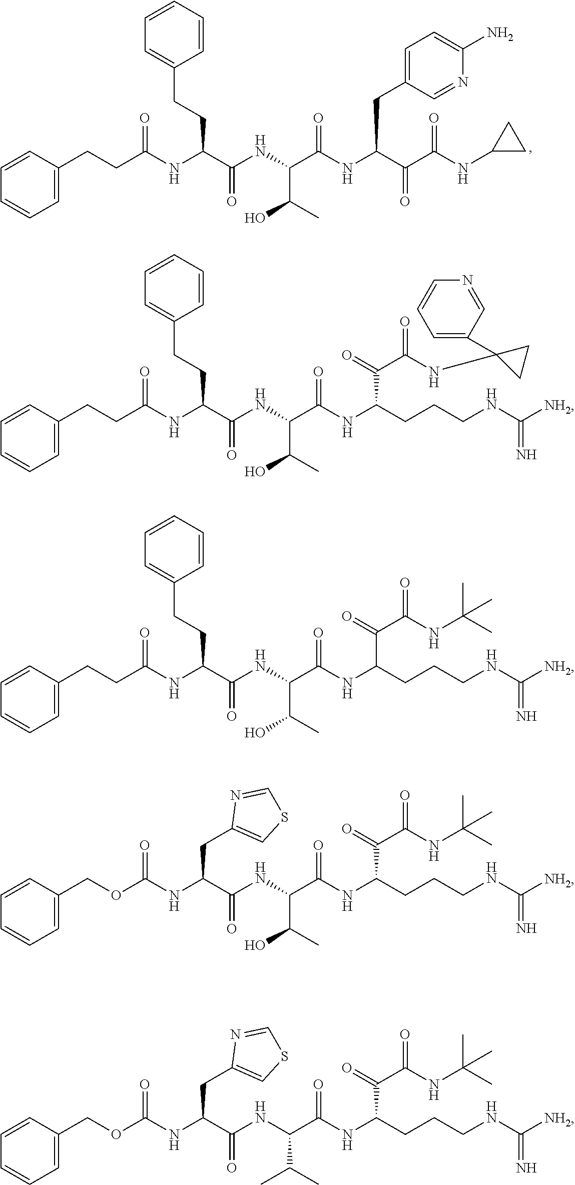

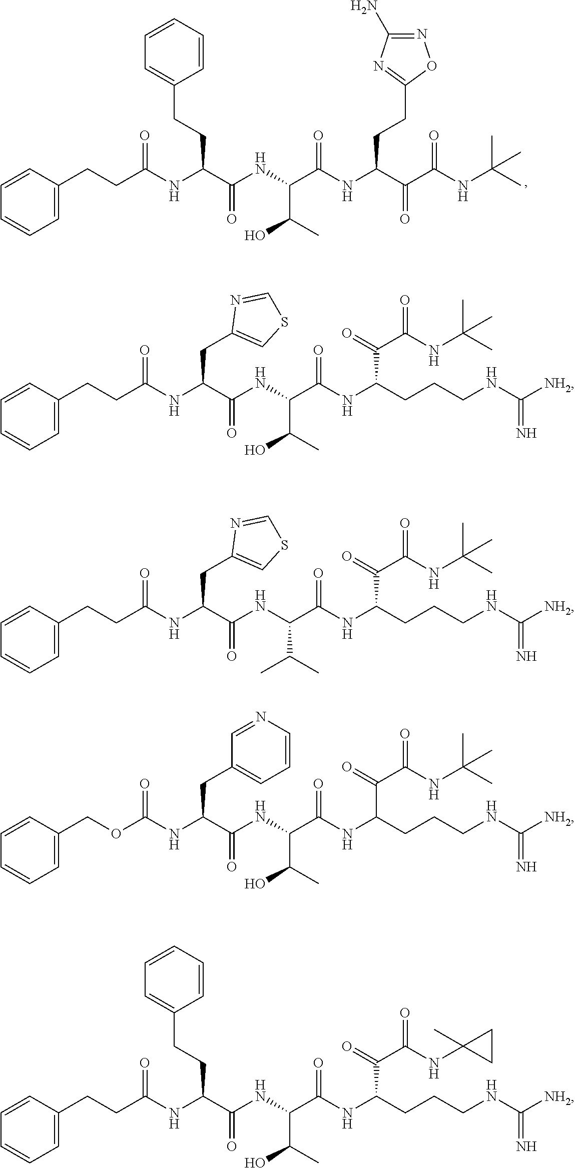

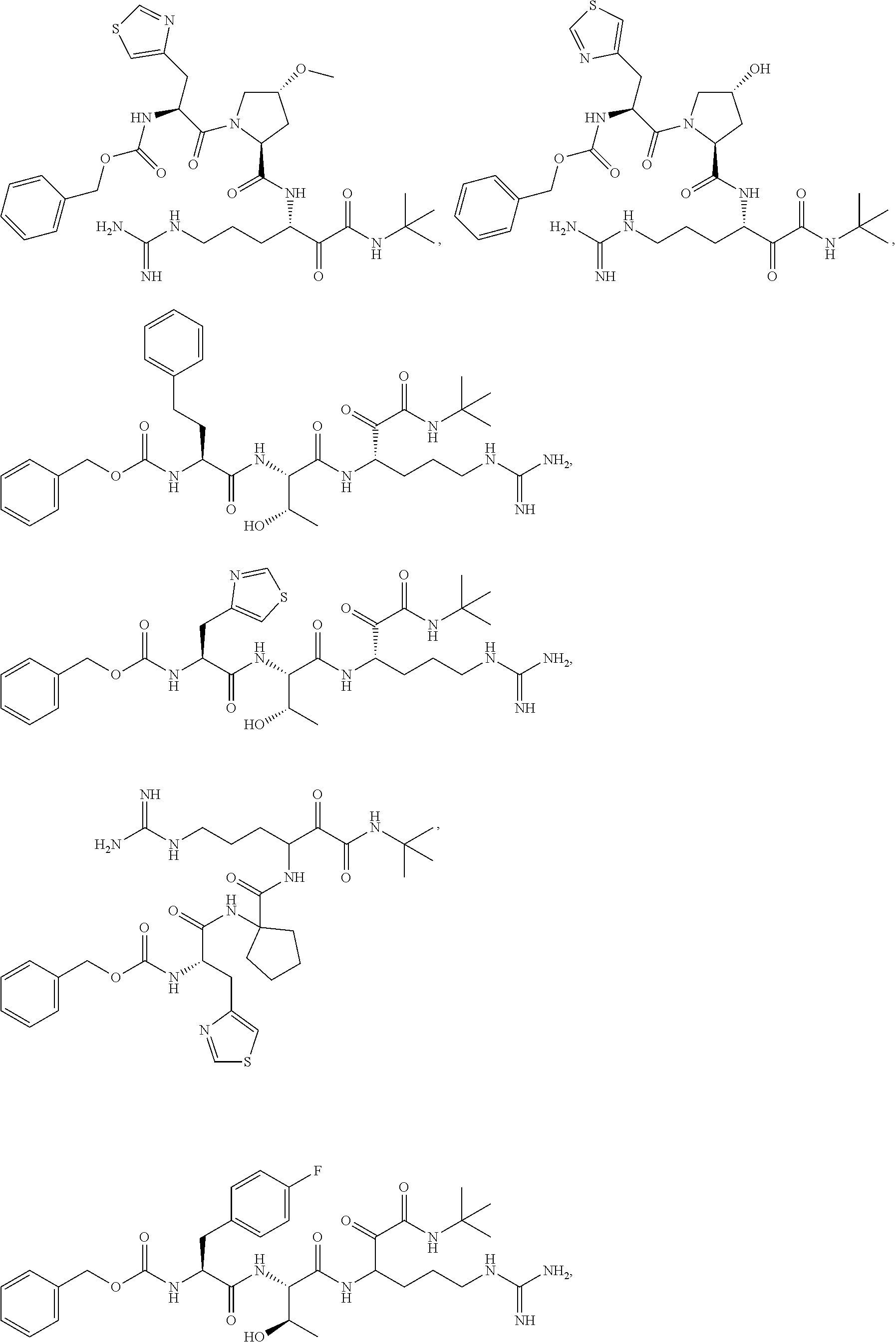

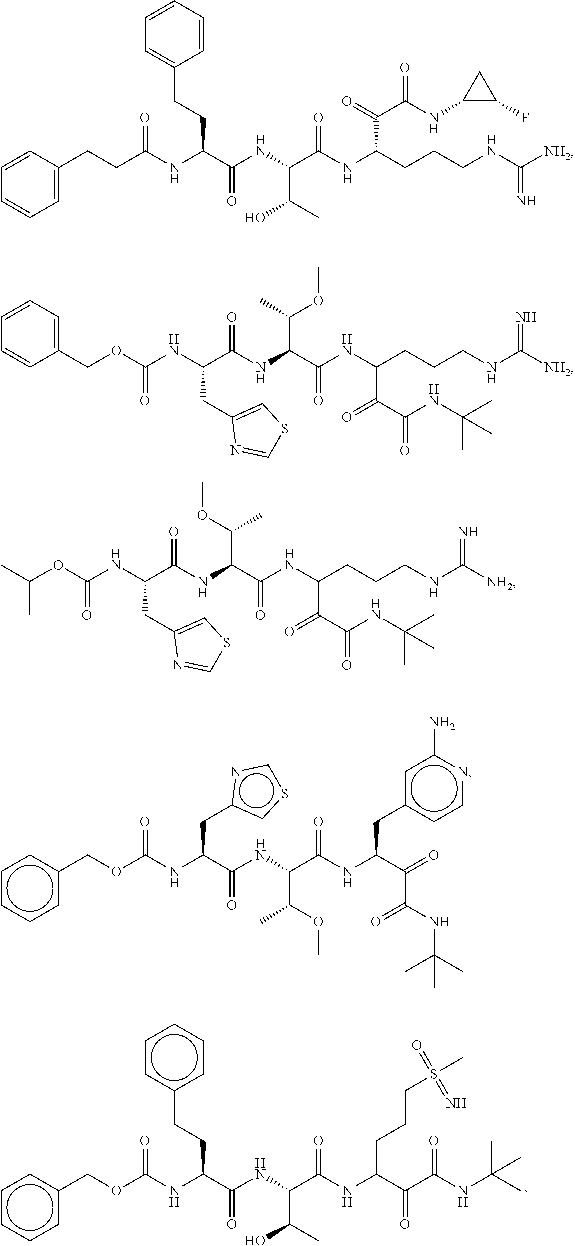

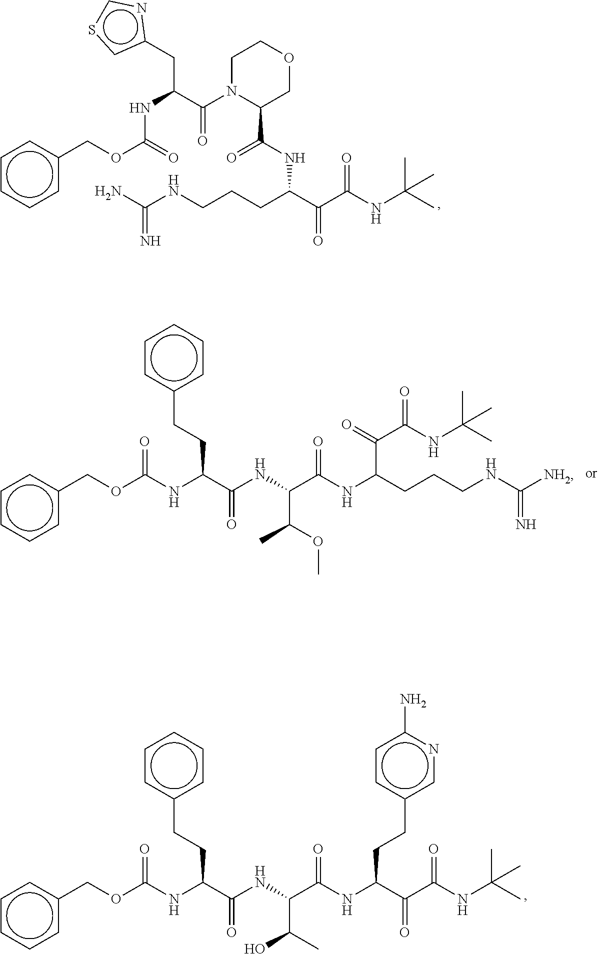

or a pharmaceutically acceptable salt thereof.

Exemplary compounds of the disclosure are set forth in Table A, below.

TABLE A
Exemplary Compounds

| | | | | UPLC | |
|---|---|---|---|---|---|
| | | | MS | RT min | |
| STRUCTURE | Mat-2 Ki | MW | (M + 1) | (method) | Synthesis |
| 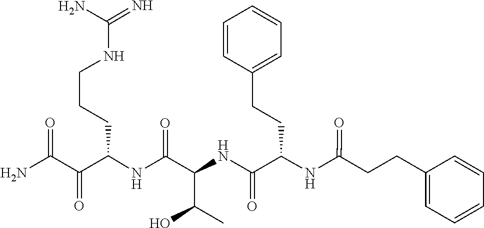 [SMILES: C[C@@H](O)[C@H](NC(=O)[C@H](CCc1ccccc1)NC(=O)CCc1ccccc1)C(=O)N[C@@H](CCCNC(=N)N)C(=O)C(N)=O] | A | 595.69 | 596.442 | 1.17 (method 1) | B (Example 2) |
| 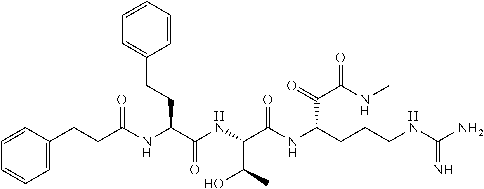 [SMILES: CNC(=O)C(=O)[C@H](CCCNC(=N)N)NC(=O)[C@@H](NC(=O)[C@H](CCc1ccccc1)NC(=O)CCc1ccccc1)[C@@H](C)O] | A | 609.72 | 610.361 | 2.21 (method 3) | A (Example 1) |
| 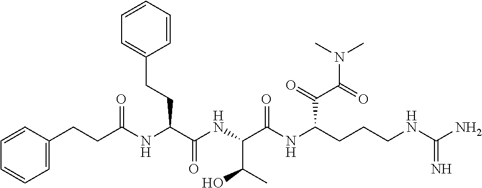 [SMILES: C[C@@H](O)[C@H](NC(=O)[C@H](CCc1ccccc1)NC(=O)CCc1ccccc1)C(=O)N[C@@H](CCCNC(=N)N)C(=O)C(=O)N(C)C] | C | 623.74 | 624.444 | 1.27 (method 1) | C (Example 3) |
| 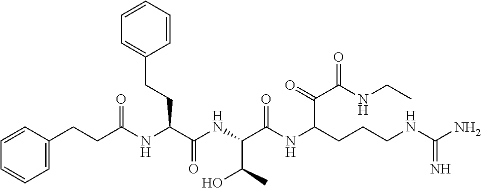 [SMILES: CCNC(=O)C(=O)C(CCCNC(=N)N)NC(=O)[C@@H](NC(=O)[C@H](CCc1ccccc1)NC(=O)CCc1ccccc1)[C@@H](C)O] | A | 623.74 | 624.268 | 2.31 (method 3) | C (Example 3) |
| 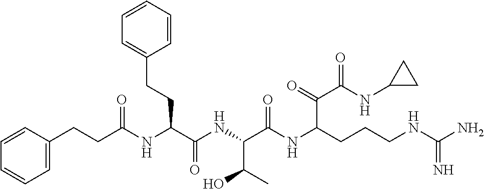 [SMILES: C[C@@H](O)[C@H](NC(=O)[C@H](CCc1ccccc1)NC(=O)CCc1ccccc1)C(=O)NC(CCCNC(=N)N)C(=O)C(=O)NC1CC1] | A | 635.75 | 636.353 | 2.38 (method 3) | A (Example 1) |
| 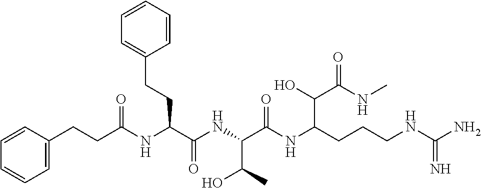 [SMILES: CNC(=O)C(O)C(CCCNC(=N)N)NC(=O)[C@@H](NC(=O)[C@H](CCc1ccccc1)NC(=O)CCc1ccccc1)[C@@H](C)O] | C | 611.73 | 612.189 | 2.17 (method 3) | C (Example 3) |
| 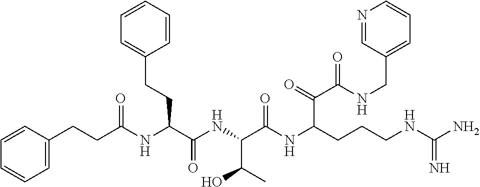 [SMILES: C[C@@H](O)[C@H](NC(=O)[C@H](CCc1ccccc1)NC(=O)CCc1ccccc1)C(=O)NC(CCCNC(=N)N)C(=O)C(=O)NCc1cccnc1] | A | 686.8 | 687.425 | 2.04 (method 3) | C (Example 3) |
| 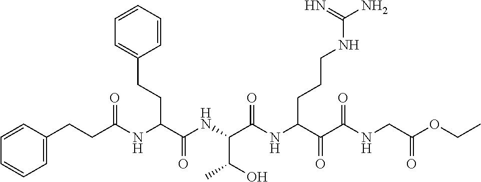 [SMILES: CCOC(=O)CNC(=O)C(=O)C(CCCNC(=N)N)NC(=O)[C@@H](NC(=O)C(CCc1ccccc1)NC(=O)CCc1ccccc1)[C@@H](C)O] | B | 681.78 | 682.193 | 2.43 (method 3) | A (Example 1) |
| 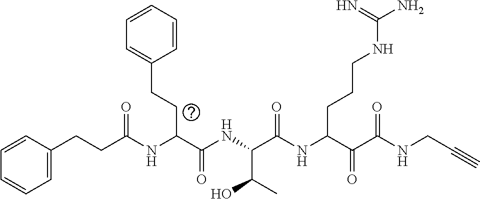 [SMILES: C#CCNC(=O)C(=O)C(CCCNC(=N)N)NC(=O)[C@@H](NC(=O)C(CCc1ccccc1)NC(=O)CCc1ccccc1)[C@@H](C)O] | B | 634.73 | 635.073 | 2.25 (method 3) | C (Example 3) |
| 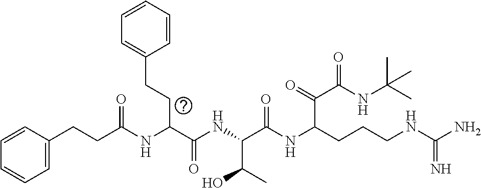 [SMILES: C[C@@H](O)[C@H](NC(=O)C(CCc1ccccc1)NC(=O)CCc1ccccc1)C(=O)NC(CCCNC(=N)N)C(=O)C(=O)NC(C)(C)C] | A | 651.8 | 650.573 | 1.54 (method 2) | A (Example 1) |
| 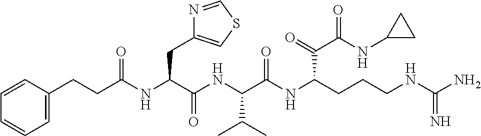 [SMILES: CC(C)[C@H](NC(=O)[C@H](Cc1cscn1)NC(=O)CCc1ccccc1)C(=O)N[C@@H](CCCNC(=N)N)C(=O)C(=O)NC1CC1] | A | 626.77 | 627.269 | 2.05 (method 3) | A (Example 1) |
| 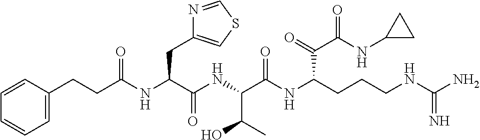 [SMILES: C[C@@H](O)[C@H](NC(=O)[C@H](Cc1cscn1)NC(=O)CCc1ccccc1)C(=O)N[C@@H](CCCNC(=N)N)C(=O)C(=O)NC1CC1] | A | 628.74 | 629.17 | 1.78 (method 3) | A (Example 1) |
| 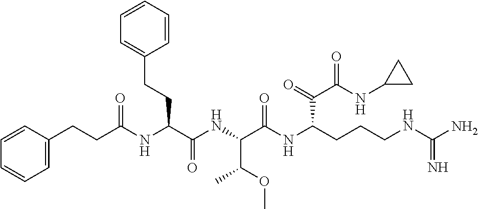 [SMILES: CO[C@H](C)[C@H](NC(=O)[C@H](CCc1ccccc1)NC(=O)CCc1ccccc1)C(=O)N[C@@H](CCCNC(=N)N)C(=O)C(=O)NC1CC1] | A | 649.78 | 650.38 | 2.5 (method 3) | A (Example 1) |
| 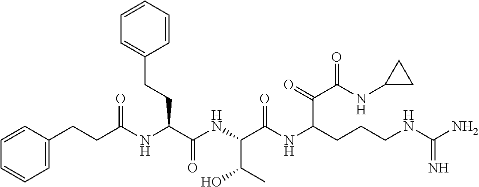 [SMILES: C[C@H](O)[C@H](NC(=O)[C@H](CCc1ccccc1)NC(=O)CCc1ccccc1)C(=O)NC(CCCNC(=N)N)C(=O)C(=O)NC1CC1] | A | 635.75 | 636.305 | 1.38 (method 1) | A (Example 1) |
| 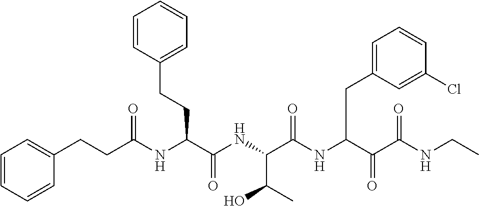 [SMILES: CCNC(=O)C(=O)C(Cc1cccc(Cl)c1)NC(=O)[C@@H](NC(=O)[C@H](CCc1ccccc1)NC(=O)CCc1ccccc1)[C@@H](C)O] | C | 649.18 | 671.188/ 673.089 (M + Na) | 3.09 (method 3) | A (Example 1) |
| 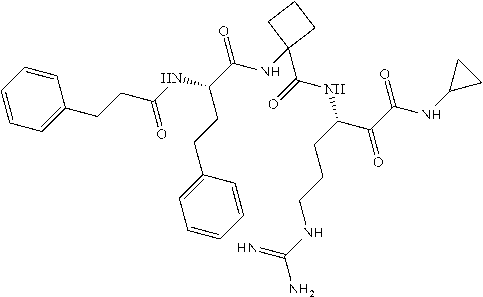 [SMILES: N=C(N)NCCC[C@H](NC(=O)C1(NC(=O)[C@H](CCc2ccccc2)NC(=O)CCc2ccccc2)CCC1)C(=O)C(=O)NC1CC1] | A | 631.77 | 632.615 | 1.37 (method 1) | D (example 4) |
| 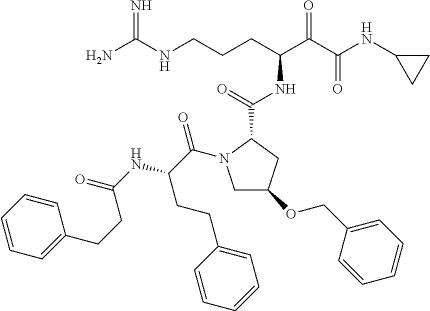 [SMILES: N=C(N)NCCC[C@H](NC(=O)[C@@H]1C[C@@H](OCc2ccccc2)CN1C(=O)[C@H](CCc1ccccc1)NC(=O)CCc1ccccc1)C(=O)C(=O)NC1CC1] | A | 737.89 | 738.397 | 1.76 (method 1); 2.95 (method 3) | D (example 4) |
| 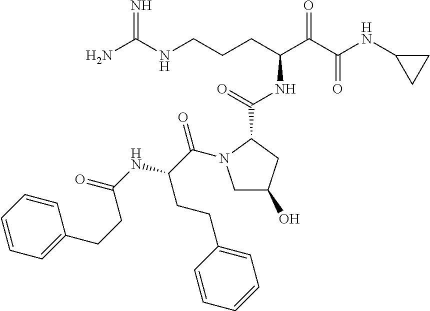 [SMILES: N=C(N)NCCC[C@H](NC(=O)[C@@H]1C[C@@H](O)CN1C(=O)[C@H](CCc1ccccc1)NC(=O)CCc1ccccc1)C(=O)C(=O)NC1CC1] | A | 647.76 | 648.35 | 1.35 (method 1); 2.91 (method 3) | D (example 4) |
| 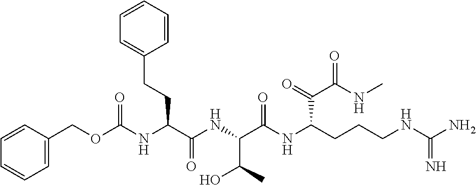 [SMILES: CNC(=O)C(=O)[C@H](CCCNC(=N)N)NC(=O)[C@@H](NC(=O)[C@H](CCc1ccccc1)NC(=O)OCc1ccccc1)[C@@H](C)O] | A | 611.69 | 612.262 | 1.35 (method 1) | D (example 4) |
| 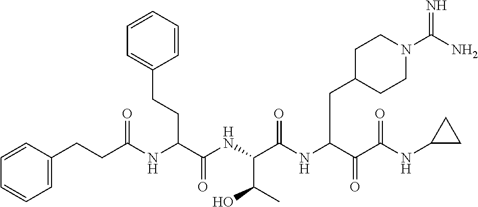 [SMILES: C[C@@H](O)[C@H](NC(=O)C(CCc1ccccc1)NC(=O)CCc1ccccc1)C(=O)NC(CC1CCN(C(=N)N)CC1)C(=O)C(=O)NC1CC1] | B | 675.82 | 676.291 | 2.38 (method 3) | E (Example 5) |
| 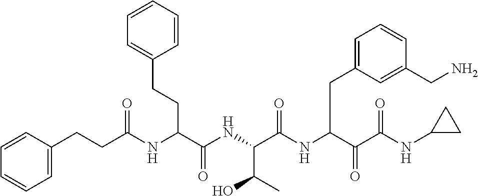 [SMILES: C[C@@H](O)[C@H](NC(=O)C(CCc1ccccc1)NC(=O)CCc1ccccc1)C(=O)NC(Cc1cccc(CN)c1)C(=O)C(=O)NC1CC1] | B | 655.78 | 656.382 | 2.43 (method 3) | A (Example 1) |
| 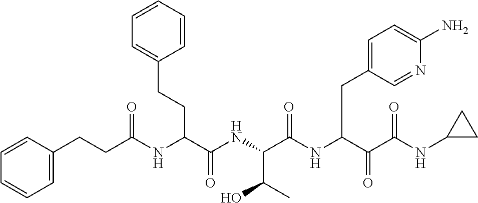 [SMILES: C[C@@H](O)[C@H](NC(=O)C(CCc1ccccc1)NC(=O)CCc1ccccc1)C(=O)NC(Cc1ccc(N)nc1)C(=O)C(=O)NC1CC1] | C | 642.74 | 643.324 | 1.39 (method 1); 2.34 (method 3) | A (Example 1) |
| 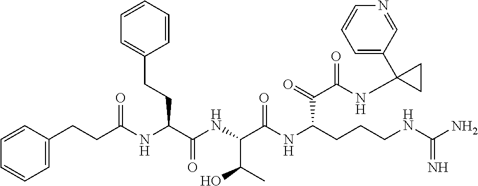 [SMILES: C[C@@H](O)[C@H](NC(=O)[C@H](CCc1ccccc1)NC(=O)CCc1ccccc1)C(=O)N[C@@H](CCCNC(=N)N)C(=O)C(=O)NC1(c2cccnc2)CC1] | A | 712.84 | 713.377 | 1.23 (method 1); 2.10 (method 3) | C (Example 3) |
| 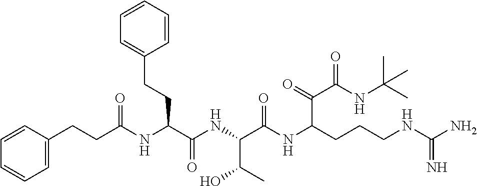 [SMILES: C[C@H](O)[C@H](NC(=O)[C@H](CCc1ccccc1)NC(=O)CCc1ccccc1)C(=O)NC(CCCNC(=N)N)C(=O)C(=O)NC(C)(C)C] | A | 651.8 | 652.28 | 1.77 (method 2) | A (Example 1) |
| 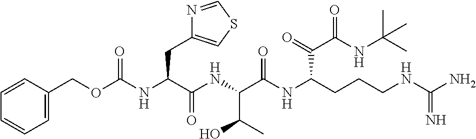 [SMILES: C[C@@H](O)[C@H](NC(=O)[C@H](Cc1cscn1)NC(=O)OCc1ccccc1)C(=O)N[C@@H](CCCNC(=N)N)C(=O)C(=O)NC(C)(C)C] | A | 646.76 | 647.378 | 2.13 (method 3) | A (Example 1) |
| 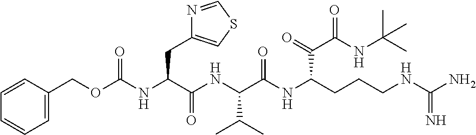 [SMILES: CC(C)[C@H](NC(=O)[C@H](Cc1cscn1)NC(=O)OCc1ccccc1)C(=O)N[C@@H](CCCNC(=N)N)C(=O)C(=O)NC(C)(C)C] | A | 644.79 | 645.304 | 2.49 (method 3) | A (Example 1) |
| 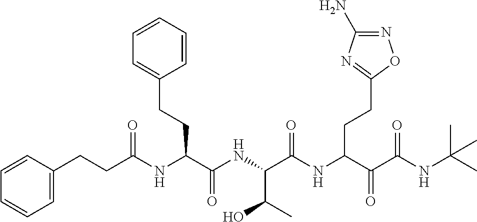 [SMILES: C[C@@H](O)[C@H](NC(=O)[C@H](CCc1ccccc1)NC(=O)CCc1ccccc1)C(=O)NC(CCc1nc(N)no1)C(=O)C(=O)NC(C)(C)C] | C | 663.76 | 664.085 | 2.79 (method 3) | E (Example 5) |
| 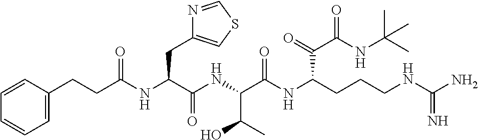 [SMILES: C[C@@H](O)[C@H](NC(=O)[C@H](Cc1cscn1)NC(=O)CCc1ccccc1)C(=O)N[C@@H](CCCNC(=N)N)C(=O)C(=O)NC(C)(C)C] | A | 644.79 | 645.277 | 2.06 (method 3) | A (Example 1) |
| 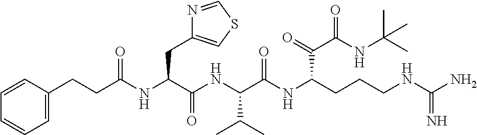 [SMILES: CC(C)[C@H](NC(=O)[C@H](Cc1cscn1)NC(=O)CCc1ccccc1)C(=O)N[C@@H](CCCNC(=N)N)C(=O)C(=O)NC(C)(C)C] | A | 642.81 | 243.276 | 2.41 (method 3) | A (Example 1) |
| 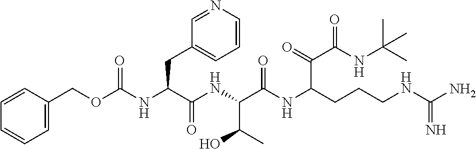 [SMILES: C[C@@H](O)[C@H](NC(=O)[C@H](Cc1cccnc1)NC(=O)OCc1ccccc1)C(=O)NC(CCCNC(=N)N)C(=O)C(=O)NC(C)(C)C] | A | 640.73 | 641.276 | 1.65 (method 3) | A (Example 1) |
| 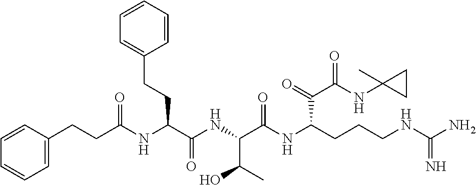 [SMILES: C[C@@H](O)[C@H](NC(=O)[C@H](CCc1ccccc1)NC(=O)CCc1ccccc1)C(=O)N[C@@H](CCCNC(=N)N)C(=O)C(=O)NC1(C)CC1] | A | 649.78 | 650.366 | 1.45 (method 1); 2.37 (method 3) | B (Example 2) |
| 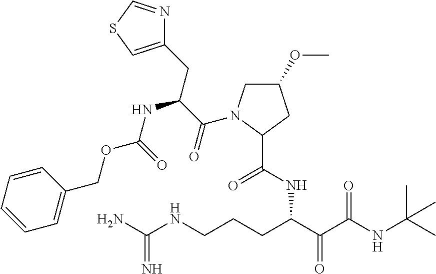 [SMILES: CO[C@@H]1CC(C(=O)N[C@@H](CCCNC(=N)N)C(=O)C(=O)NC(C)(C)C)N(C(=O)[C@H](Cc2cscn2)NC(=O)OCc2ccccc2)C1] | A | 672.8 | 673.313 | 1.39 (method 1) | D (example 4) |
| 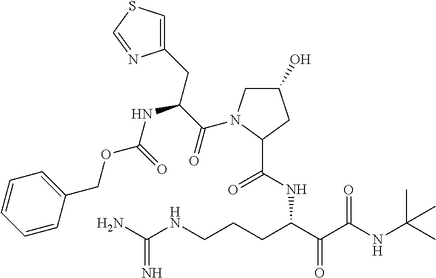 [SMILES: CC(C)(C)NC(=O)C(=O)[C@H](CCCNC(=N)N)NC(=O)C1C[C@@H](O)CN1C(=O)[C@H](Cc1cscn1)NC(=O)OCc1ccccc1] | A | 658.77 | 659.297 | 1.22 (method 1) | D (example 4) |
| 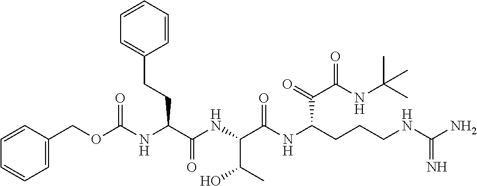 [SMILES: C[C@H](O)[C@H](NC(=O)[C@H](CCc1ccccc1)NC(=O)OCc1ccccc1)C(=O)N[C@@H](CCCNC(=N)N)C(=O)C(=O)NC(C)(C)C] | A | 653.77 | 654.282 | 2.71 (method 3) | A (Example 1) |
| 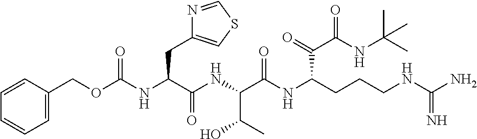 [SMILES: C[C@H](O)[C@H](NC(=O)[C@H](Cc1cscn1)NC(=O)OCc1ccccc1)C(=O)N[C@@H](CCCNC(=N)N)C(=O)C(=O)NC(C)(C)C] | A | 646.76 | 647.621 | 1.19 (method 3) | A (Example 1) |
| 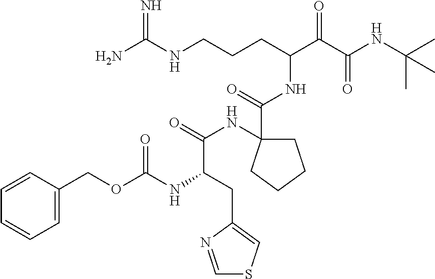 [SMILES: CC(C)(C)NC(=O)C(=O)C(CCCNC(=N)N)NC(=O)C1(NC(=O)[C@H](Cc2cscn2)NC(=O)OCc2ccccc2)CCCC1] | A | 656.8 | 657.182 | 2.59 (method 3) | A (Example 1) |
| 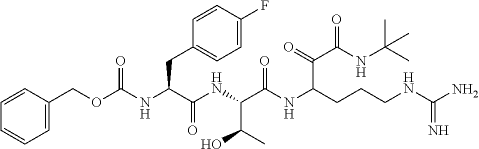 [SMILES: C[C@@H](O)[C@H](NC(=O)[C@H](Cc1ccc(F)cc1)NC(=O)OCc1ccccc1)C(=O)NC(CCCNC(=N)N)C(=O)C(=O)NC(C)(C)C] | A | 657.73 | 658.283 | 2.67 (method 3) | A (Example 1) |
| 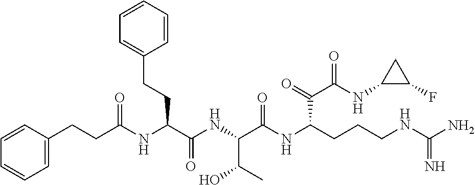 [SMILES: C[C@H](O)[C@H](NC(=O)[C@H](CCc1ccccc1)NC(=O)CCc1ccccc1)C(=O)N[C@@H](CCCNC(=N)N)C(=O)C(=O)N[C@@H]1C[C@@H]1F] | A | 653.74 | 654.341 | 1.36 (method 1); 2.39 (method 3) | B (Example 2) |
| 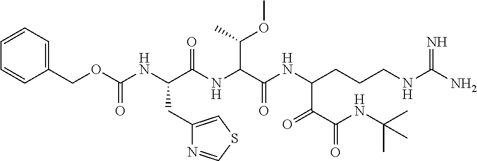 [SMILES: CO[C@@H](C)C(NC(=O)[C@H](Cc1cscn1)NC(=O)OCc1ccccc1)C(=O)NC(CCCNC(=N)N)C(=O)C(=O)NC(C)(C)C] | A | 660.78 | 661.284 | 2.33 (method 3) | D (example 4) |
| 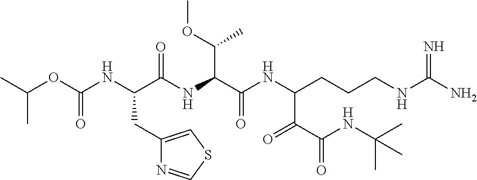 [SMILES: CO[C@H](C)[C@H](NC(=O)[C@H](Cc1cscn1)NC(=O)OC(C)C)C(=O)NC(CCCNC(=N)N)C(=O)C(=O)NC(C)(C)C] | A | 612.74 | 613.263 | 2.10 (method 3) | D (example 4) |
| 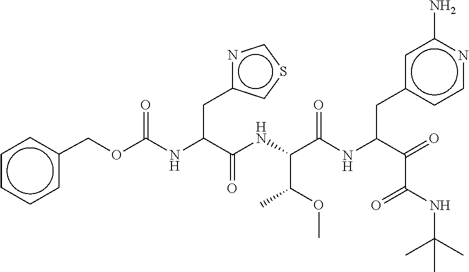 | A | 667.78 | 669 | 2.22 (method 3) | A (Example 1) |
| 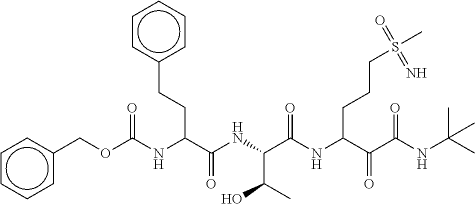 | C | 673.82 | 674.322 | 1.80 (method 1); 1.58 (method 2) | A (Example 1) |
| 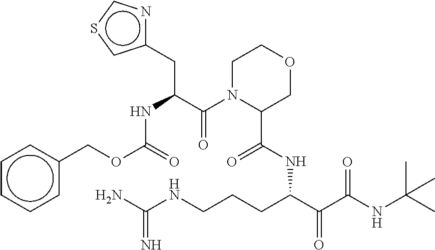 | A | 658.77 | 659.183 | 1.60 (method 2) | A (Example 1) |
| 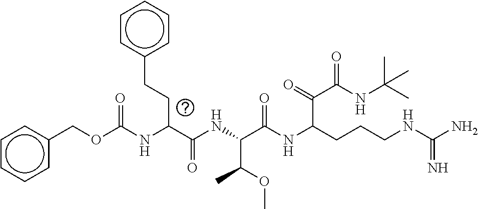 | A | 667.8 | 668.378 | 1.84 (method 2) | D (example 4) |
| 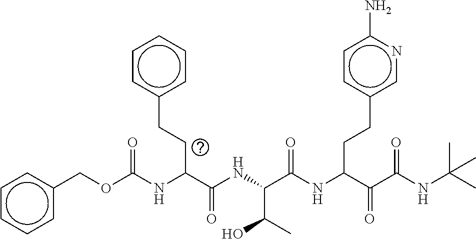 | B | 674.79 | 675.35 | 1.83 (method 1); 1.61 (method 2) | A (Example 1) |

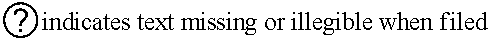

Mat-2 Ki ranges: A is “≤0.1 uM”; B is “0.1 uM to ≤1 uM”; C is “≥1 uM”
Definition of % inhibition (I) range at 1 uM:

In some embodiments, the present disclosure provides a compound set forth in Table A, above, or a pharmaceutically acceptable salt thereof.

### Uses, Formulation and Administration:

### Pharmaceutically Acceptable Compositions

In some embodiments, provided are pharmaceutical compositions comprising a compound, or a pharmaceutically acceptable salt thereof, as described herein.

According to another embodiment, the present disclosure provides a composition comprising a compound of the present disclosure or a pharmaceutically acceptable derivative thereof and a pharmaceutically acceptable carrier, adjuvant, or vehicle. In some embodiments, provided are pharmaceutical compositions comprising a compound, or a pharmaceutically acceptable salt thereof, as described herein and a pharmaceutically acceptable carrier, adjuvant, or vehicle. In some embodiments, provided are pharmaceutical compositions comprising a compound, or a pharmaceutically acceptable salt thereof, as described herein and a pharmaceutically acceptable carrier. In some embodiments, provided are pharmaceutical compositions comprising a compound, or a pharmaceutically acceptable salt thereof, as described herein and a pharmaceutically acceptable adjuvant. In some embodiments, provided are pharmaceutical compositions comprising a compound, or a pharmaceutically acceptable salt thereof, as described herein and a pharmaceutically acceptable vehicle.

The amount of compound in compositions of the present disclosure is such that is effective to measurably inhibit Matriptase 2, or a mutant thereof, in a biological sample or in a patient. In certain embodiments, the amount of compound in compositions of the present disclosure is such that is effective to measurably inhibit Matriptase 2, or a mutant thereof, in a biological sample or in a patient. In certain embodiments, a composition of the present disclosure is formulated for administration to a patient in need of such composition. In some embodiments, a composition of the present disclosure is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present disclosure that, upon administration to a recipient, is capable of providing, either directly or indirectly, a compound of the present disclosure or an inhibitorily active metabolite or residue thereof.

As used herein, the term “inhibitorily active metabolite or residue thereof” means that a metabolite or residue thereof is also an inhibitor of Matriptase 2, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of the present disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of the present disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of the present disclosure may be administered in the form of suppositories for rectal administration. These can be prepared by mixing the agent with a suitable non-irritating excipient that is solid at room temperature but liquid at rectal temperature and, therefore, will melt in the rectum to release the drug. Such materials include cocoa butter, beeswax and polyethylene glycols.

Pharmaceutically acceptable compositions of the present disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of the present disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of the present disclosure are formulated for oral administration. Such formulations may be administered with or without food. In some embodiments, pharmaceutically acceptable compositions of the present disclosure are administered without food. In other embodiments, pharmaceutically acceptable compositions of the present disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the inhibition of Matriptase 2, or a mutant thereof.

The activity of a compound utilized in the present disclosure as an inhibitor of Matriptase 2, or a mutant thereof, may be assayed in vitro, in vivo or in a cell line. In vitro assays include assays that determine inhibition of Matriptase 2, or a mutant thereof. Alternate in vitro assays quantitate the ability of the inhibitor to bind to Matriptase 2, or a mutant thereof. Detailed conditions for assaying a compound utilized in the present disclosure as an inhibitor of Matriptase 2, or a mutant thereof, are set forth in the Examples below.

As used herein, the terms “treatment,” “treat,” or “treating” of any disease or disorder refers, in some embodiments, to ameliorating the disease or disorder (i.e., arresting or reducing the development of the disease or at least one of the clinical symptoms thereof). In another embodiment, “treating,” “treat,” or “treatment” refers to ameliorating at least one physical parameter, which may not be discernible by the subject. In yet another embodiment, “treatment,” “treat,” or “treating” refers to modulating the disease or disorder, either physically (e.g., stabilization of a discernible symptom), physiologically (e.g., stabilization of a physical parameter), or both. In yet another embodiment, “treatment,” “treat,” or “treating” refers to delaying the onset of the disease or disorder.

As used herein, the terms “low hepcidin” disorders, diseases, and/or conditions as used herein means any disease or other deleterious condition in which absolute or relative hepcidin deficiency is known to play a role, or in which an increase in hepcidin may be therapeutically useful.

Provided compounds are inhibitors of Matriptase 2, or a mutant thereof, and are therefore useful for treating low hepcidin disorders, diseases, and/or conditions. Accordingly, in certain embodiments, the present disclosure provides a method for treating a low hepcidin disorder, disease, and/or condition, comprising the step of administering to a patient in need thereof a compound of the present disclosure, or pharmaceutically acceptable composition thereof.

Without wishing to be bound by any specific theory, inhibition of Matriptase-2 has been found to lead to increased hepcidin production by the liver. Accordingly, in some embodiments, the present disclosure provides a method for increasing hepcidin production by the liver in a patient, comprising the step of administering to the patient a compound of the present disclosure, or pharmaceutically acceptable composition thereof. In some embodiments, the present disclosure provides a method for treating absolute and/or relative hepcidin deficiency in a patient, comprising the step of administering to the patient a compound of the present disclosure, or pharmaceutically acceptable composition thereof. In some embodiments, the present disclosure provides a method for treating hepcidin underproduction in a patient, comprising the step of administering to the patient a compound of the present disclosure, or pharmaceutically acceptable composition thereof. In some embodiments, the present disclosure provides a method for treating excess or increased iron absorption or accumulation in a patient, comprising the step of administering to the patient a compound of the present disclosure, or pharmaceutically acceptable composition thereof in order to increase hepcidin production by the liver. In some embodiments, the present disclosure provides a method for treating ineffective erythropoiesis in a patient, comprising the step of administering to the patient a compound of the present disclosure, or pharmaceutically acceptable composition thereof.

In some embodiments, the present disclosure provides a method for treating one or more iron overload disorder, disease, and/or condition, comprising the step of administering to a patient in need thereof a compound of the present disclosure, or pharmaceutically acceptable composition thereof.

As used herein, the term “iron overload disorder, disease, and/or condition” refers to a condition, disease, or disorder associated with excessive iron levels or iron overload. Large amounts of free iron in the bloodstream can lead to cell damage, especially in the liver, heart and endocrine glands. The causes of excess iron may be genetic, for example the iron excess may be caused by a genetic condition such as hemochromatosis type 1 (classical hemochromatosis), hemochromatosis type 2A or 2B (juvenile hemochromatosis), hemochromatosis type 3, African iron overload, neonatal hemochromatosis, aceruloplasminemia, or congenital atransferrinemia. Examples of non-genetic causes of iron excess include dietary iron overload (including African iron overload), transfusional iron overload (due to a blood transfusion given to patients with thalassaemia or other congenital hematological disorders), hemodialysis, chronic liver disease (such as hepatitis C, cirrhosis, non-alcoholic steatohepatitis), porphyria cutanea tarda, post-portacaval shunting, dysmetabolic overload syndrome, iron tablet overdose (such as that caused by consumption by children of iron tablets intended for adults), or any other cause of acute or chronic iron overload.

In some embodiments, an iron overload disorder, disease, and/or condition is Hemochromatosis Type 1. In some embodiments, an iron overload disorder, disease, and/or condition is Hemochromatosis Type 2a. In some embodiments, an iron overload disorder, disease, and/or condition is Hemochromatosis Type 2b. In some embodiments, an iron overload disorder, disease, and/or condition is Hemochromatosis Type 3.

In some embodiments, an iron overload disorder, disease, and/or condition is hepcidin deficiency. In some embodiments, an iron overload disorder, disease, and/or condition is Transfusional iron overload. In some embodiments, an iron overload disorder, disease, and/or condition is African iron overload. In some embodiments, an iron overload disorder, disease, and/or condition is Iron overload cardiomyopathy.

In some embodiments, the present disclosure provides a method for treating one or more iron loading anemia, comprising the step of administering to a patient in need thereof a compound of the present disclosure, or pharmaceutically acceptable composition thereof. In some embodiments, an Iron Loading Anemia is beta thalassemia, HbE/beta thalassemia, or other variants thereof, including but not limited to: thalassemia major, thalassemia intermedia, thalassemia minor, non-transfusion dependent thalassemia, and transfusion-dependent thalassemia. In some embodiments, an iron loading anemia is associated with, and/or caused by, alpha thalassemia. In some embodiments, an iron loading anemia is congenital dyserythropoietic anemia Type I and/or Type II. In some embodiments, an iron loading anemia is pyruvate kinase deficiency. In some embodiments, an iron loading anemia is myelodyplasia including but not limited to myelodysplastic syndrome (MDS), RARS and/or SF3B1 associated MDS.

In some embodiments, the present disclosure provides a method for treating one or more hematological disease, disorder, and/or condition, comprising the step of administering to a patient in need thereof a compound of the present disclosure, or pharmaceutically acceptable composition thereof. In some embodiments, a hematological disease, disorder, and/or condition is sickle cell disease. In some embodiments, a hematological disease, disorder, and/or condition is sickle cell anemia. In some embodiments, a hematological disease, disorder, and/or condition is polycythemia vera. In some embodiments, a hematological disease, disorder, and/or condition is sideroblastic anemia. In some embodiments, a hematological disease, disorder, and/or condition is bone marrow transplantation.

In some embodiments, the present disclosure provides a method for treating one or more liver diseases, comprising the step of administering to a patient in need thereof a compound of the present disclosure or pharmaceutically acceptable composition thereof. In some embodiments, a liver disease is Hepatitis B. In some embodiments, a liver disease is Hepatitis C or other forms of viral hepatitis. In some embodiments, a liver disease is alcoholic liver disease. In some embodiments, a liver disease is cirrhosis of the liver. In some embodiments, a liver disease is hepatocellular carcinoma. In some embodiments, a liver disease is non-alcoholic steatohepatitis (NASH).

In some embodiments, the present disclosure provides a method for treating one or more metabolic disease, comprising the step of administering to a patient in need thereof a compound of the present disclosure or pharmaceutically acceptable composition thereof. In some embodiments, a metabolic disease is metabolic syndrome. In some embodiments, a metabolic disease is insulin resistance. In some embodiments, a metabolic disease is Type II diabetes. In some embodiments, a metabolic disease is porphyria. In some embodiments, a metabolic disease is porphyria cutanea tarda. In some embodiments, a metabolic disease is Wilson's Disease. In some embodiments, a metabolic disease is acute iron overdose.

In some embodiments, the present disclosure provides a method for treating one or more neurodegenerative disorders, comprising the step of administering to a patient in need thereof a compound of the present disclosure or pharmaceutically acceptable composition thereof. In some embodiments, a neurodegenerative disorder is selected from the group consisting of Huntington's Disease (HD); Parkinson's Disease (PD); amyotrophic lateral sclerosis (ALS); frontotemporal dementia (FTD); corticobasal degeneration (CBD); progressive supranuclear palsy (PSP); dementia with Lewy Bodies (DLB); and multiple sclerosis (MS).

In some embodiments, the present disclosure provides a method for treating one or more infectious diseases, comprising the step of administering to a patient in need thereof a compound of the present disclosure or pharmaceutically acceptable composition thereof. In some embodiments, an infectious disease is a siderophilic infection.

In some embodiments, the present disclosure provides use of a compound of the present disclosure, or a pharmaceutically acceptable salt thereof, or the pharmaceutical composition of the present disclosure for treating a low hepcidin disorder, disease, and/or condition in a subject. In some embodiments, the present disclosure provides of a compound of the present disclosure, or a pharmaceutically acceptable salt thereof, or the pharmaceutical composition of the present disclosure, in the manufacture of a medicament for treating a low hepcidin disorder, disease, and/or condition in a subject. In some embodiments, the present disclosure provides a compound of the present disclosure, or a pharmaceutically acceptable salt thereof, or the pharmaceutical composition of the present disclosure, in use for treating a low hepcidin disorder, disease, and/or condition in a subject.

In some embodiments, the present disclosure provides use of a compound of the present disclosure, or a pharmaceutically acceptable salt thereof, or the pharmaceutical composition of the present disclosure for increasing hepcidin production in a subject. In some embodiments, the present disclosure provides use of a compound of the present disclosure, or a pharmaceutically acceptable salt thereof, or the pharmaceutical composition of the present disclosure, in the manufacture of a medicament for increasing hepcidin production in a subject. In some embodiments, the present disclosure provides a compound of the present disclosure, or a pharmaceutically acceptable salt thereof, or the pharmaceutical composition of the present disclosure, in use for increasing hepcidin production in a subject.

In some embodiments, the present disclosure provides use of a compound of the present disclosure, or a pharmaceutically acceptable salt thereof, or the pharmaceutical composition of the present disclosure for treating an iron overload disorder, disease, and/or condition in a subject.

In some embodiments, the present disclosure provides use of a compound of the present disclosure, or a pharmaceutically acceptable salt thereof, or the pharmaceutical composition of the present disclosure, in the manufacture of a medicament for treating an iron overload disorder, disease, and/or condition in a subject. In some embodiments, the present disclosure provides a compound of the present disclosure, or a pharmaceutically acceptable salt thereof, or the pharmaceutical composition of the present disclosure, in use for treating an iron overload disorder, disease, and/or condition in a subject. In some embodiments, the iron overload disorder, disease, and/or condition is selected from the group consisting of hemochromatosis Type 1, 2a, 2b and 3 (hemochromatosis, Hfe hemochromatosis (Type 1), juvenile hemochromatosis (types 2a and 2b),), hepcidin deficiency, transfusional iron overload, African iron overload, and iron overload cardiomyopathy.

In some embodiments, the present disclosure provides use of a compound of the present disclosure, or a pharmaceutically acceptable salt thereof, or the pharmaceutical composition of the present disclosure for treating an iron loading anemia in a subject. In some embodiments, the present disclosure provides use of a compound of the present disclosure, or a pharmaceutically acceptable salt thereof, or the pharmaceutical composition of the present disclosure, in the manufacture of a medicament for treating an iron loading anemia in a subject. In some embodiments, the present disclosure provides a compound of the present disclosure, or a pharmaceutically acceptable salt thereof, or the pharmaceutical composition of the present disclosure, in use for treating an iron loading anemia in a subject. In some embodiments, the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia, congenital dyserythropoietic anemias (Type I and Type II), pyruvate kinase deficiency, and myelodysplasia (such as myelodysplastic syndrome, and RARS SF3B1 associated MDS).

In some embodiments, the present disclosure provides use of a compound of the present disclosure, or a pharmaceutically acceptable salt thereof, or the pharmaceutical composition of the present disclosure for treating a hematological disease, disorder, and/or condition in a subject. In some embodiments, the present disclosure provides use of a compound of the present disclosure, or a pharmaceutically acceptable salt thereof, or the pharmaceutical composition of the present disclosure, in the manufacture of a medicament for treating a hematological disease, disorder, and/or condition in a subject. In some embodiments, the present disclosure provides a compound of the present disclosure, or a pharmaceutically acceptable salt thereof, or the pharmaceutical composition of the present disclosure, in use for treating a hematological disease, disorder, and/or condition in a subject. In some embodiments, the hematological disease, disorder, and/or condition is selected from the group consisting of sickle cell disease (such as sickle cell anemia), polycythemia vera, sideroblastic anemia, and bone marrow transplantation.

In some embodiments, the present disclosure provides use of a compound of the present disclosure, or a pharmaceutically acceptable salt thereof, or the pharmaceutical composition of the present disclosure for treating a liver disease in a subject in a subject. In some embodiments, the present disclosure provides use of a compound of the present disclosure, or a pharmaceutically acceptable salt thereof, or the pharmaceutical composition of the present disclosure, in the manufacture of a medicament for treating a liver disease in a subject. In some embodiments, the present disclosure provides a compound of the present disclosure, or a pharmaceutically acceptable salt thereof, or the pharmaceutical composition of the present disclosure, in use for treating a liver disease in a subject in a subject. In some embodiments, the liver disease is selected from the group consisting of Hepatitis B, Hepatitis C, alcoholic liver disease, cirrhosis of the liver, epahtocellular carcinoma, and non-alcoholic steatohepatitis (NASH). In some embodiments, the liver disease is Hepatitis B. In some embodiments, the liver disease is Hepatitis C. In some embodiments, the liver disease is alcoholic liver disease. In some embodiments, the liver disease is cirrhosis of the liver, epahtocellular carcinoma. In some embodiments, the liver disease is non-alcoholic steatohepatitis (NASH).

In some embodiments, the present disclosure provides use of a compound of the present disclosure, or a pharmaceutically acceptable salt thereof, or the pharmaceutical composition of the present disclosure, for treating a metabolic disease in a subject. In some embodiments, the present disclosure provides use of a compound of the present disclosure, or a pharmaceutically acceptable salt thereof, or the pharmaceutical composition of the present disclosure, in the manufacture of a medicament for treating a metabolic disease in a subject. In some embodiments, the present disclosure provides a compound of the present disclosure, or a pharmaceutically acceptable salt thereof, or the pharmaceutical composition of the present disclosure, in use for treating a metabolic disease in a subject. In some embodiments, the metabolic disease is selected from the group consisting of metabolic syndrome, insulin resistance, Type II diabetes, porphyria, porphyria cutanea tarda, Wilson's Disease, and acute iron overdose. In some embodiments, the metabolic disease is metabolic syndrome. In some embodiments, the metabolic disease is insulin resistance. In some embodiments, the metabolic disease is Type II diabetes. In some embodiments, the metabolic disease is porphyria. In some embodiments, the metabolic disease is porphyria cutanea tarda. In some embodiments, the metabolic disease is Wilson's Disease. In some embodiments, the metabolic disease is acute iron overdose.

In some embodiments, the present disclosure provides use of a compound of the present disclosure, or a pharmaceutically acceptable salt thereof, or the pharmaceutical composition of the present disclosure for treating a neurodegenerative disorder in a subject. In some embodiments, the present disclosure provides use of a compound of the present disclosure, or a pharmaceutically acceptable salt thereof, or the pharmaceutical composition of the present disclosure, in the manufacture of a medicament for treating a neurodegenerative disorder in a subject. In some embodiments, the present disclosure provides a compound of the present disclosure, or a pharmaceutically acceptable salt thereof, or the pharmaceutical composition of the present disclosure, in use for treating a neurodegenerative disorder in a subject.

In some embodiments, the present disclosure provides use of a compound of the present disclosure, or a pharmaceutically acceptable salt thereof, or the pharmaceutical composition of the present disclosure for treating an infectious disease in a subject. In some embodiments, the present disclosure provides use of a compound of the present disclosure, or a pharmaceutically acceptable salt thereof, or the pharmaceutical composition of the present disclosure, in the manufacture of a medicament for treating an infectious disease in a subject. In some embodiments, the present disclosure provides a compound of the present disclosure, or a pharmaceutically acceptable salt thereof, or the pharmaceutical composition of the present disclosure, in use for treating an infectious disease in a subject. In some embodiments, the infectious disease is a siderophilic infection.

In some embodiments, the present disclosure provides the subject is a subject in need thereof.

In some embodiments, the present disclosure provides use of a compound of the present disclosure, or a pharmaceutically acceptable salt thereof, or the pharmaceutical composition of the present disclosure, for inhibiting matriptase 2, or a mutant thereof, in a biological sample. In some embodiments, the present disclosure provides a compound of the present disclosure, or a pharmaceutically acceptable salt thereof, or the pharmaceutical composition of the present disclosure, in use for inhibiting matriptase 2, or a mutant thereof, in a biological sample.

In some embodiments, the compound of the present disclosure, or a pharmaceutically acceptable salt thereof, or the pharmaceutical composition of the present disclosure, is administered in an effective amount.

The compounds and compositions, according to the method or use of the present disclosure, may be administered using any amount and any route of administration effective for treating or lessening the severity of a low hepcidin disease, disorder, and/or condition, or any amount and any route of administration effective for increasing hepcidin production by the liver. The exact amount required will vary from subject to subject, depending on the species, age, and general condition of the subject, the severity of the disease or condition, the particular agent, its mode of administration, and the like. Compounds of the present disclosure are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of the present disclosure can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the present disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of the present disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of the present disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the present disclosure relates to a method of inhibiting matriptase 2 activity, or a mutant thereof, in a biological sample comprising the step of contacting said biological sample with a compound of this disclosure, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Depending upon the particular condition, or disease, to be treated, additional therapeutic agents that are normally administered to treat that condition, may also be present in the compositions of this disclosure. As used herein, additional therapeutic agents that are normally administered to treat a particular disease, or condition, are known as “appropriate for the disease, or condition, being treated.”

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents. In some embodiments, the method includes co-administering one additional therapeutic agent. In some embodiments, the method includes co-administering two or more additional therapeutic agents. In some embodiments, the combination of the disclosed compound and the additional therapeutic agent or agents acts synergistically. In some embodiments, an additional therapeutic agent is an iron chelating compound, or a pharmaceutically acceptable salt thereof. In some embodiments, an iron chelating compound, or a pharmaceutically acceptable salt thereof, is selected from the group consisting of deferasirox, deferiprone and deferoxamine.

In some embodiments, the present disclosure provides use of a compound of the present disclosure, or a pharmaceutically acceptable salt thereof, or the pharmaceutical composition of the present disclosure and one or more additional therapeutic agents for treating a disclosed disease or condition in a subject. In some embodiments, the present disclosure provides of a compound of the present disclosure, or a pharmaceutically acceptable salt thereof, or the pharmaceutical composition of the present disclosure, and one or more additional therapeutic agents in the manufacture of a medicament for treating a disclosed disease or condition in a subject. In some embodiments, the present disclosure provides a compound of the present disclosure, or a pharmaceutically acceptable salt thereof, or the pharmaceutical composition of the present disclosure, and one or more additional therapeutic agents in use for treating a disclosed disease or condition in a subject. In some embodiments, the use includes co-administering one additional therapeutic agent. In some embodiments, the use includes co-administering two or more additional therapeutic agents. In some embodiments, the combination of the disclosed compound and the additional therapeutic agent or agents acts synergistically. In some embodiments, an additional therapeutic agent is an iron chelating compound, or a pharmaceutically acceptable salt thereof. In some embodiments, an iron chelating compound, or a pharmaceutically acceptable salt thereof, is selected from the group consisting of deferasirox, deferiprone and deferoxamine. In some embodiments, the compound of the present disclosure, or the pharmaceutically acceptable salt thereof, or the pharmaceutical composition of the present disclosure is adminstered in an effective amount. In some embodiment, the additional therapeutic agent is adminstered in an effective amount.

In some embodiments, the present disclosure provides a method of treating a disclosed disease or condition comprising administering to a patient in need thereof an effective amount of a compound disclosed herein or a pharmaceutically acceptable salt thereof, and an iron chelating compound or a pharmaceutically acceptable salt thereof. In some embodiments, a patient is a patient with iron overload. In some embodiments, a patient is a patient with cardiac iron overload or iron overload related cardiomyopathy. In some embodiments, an iron chelating compound, or a pharmaceutically acceptable salt thereof, is selected from the group consisting of deferasirox, deferiprone and deferoxamine.

In some embodiments, the present disclosure provides use of a compound of the present disclosure, or a pharmaceutically acceptable salt thereof, or the pharmaceutical composition of the present disclosure and an iron chelating compound or a pharmaceutically acceptable salt thereof for treating a disclosed disease or condition in a subject. In some embodiments, the present disclosure provides of a compound of the present disclosure, or a pharmaceutically acceptable salt thereof, or the pharmaceutical composition of the present disclosure, and an iron chelating compound or a pharmaceutically acceptable salt thereof in the manufacture of a medicament for treating a disclosed disease or condition in a subject. In some embodiments, the present disclosure provides a compound of the present disclosure, or a pharmaceutically acceptable salt thereof, or the pharmaceutical composition of the present disclosure, and an iron chelating compound or a pharmaceutically acceptable salt thereof in use for treating a disclosed disease or condition in a subject. In some embodiments, a patient is a patient with cardiac iron overload or iron overload related cardiomyopathy. In some embodiments, an iron chelating compound, or a pharmaceutically acceptable salt thereof, is selected from the group consisting of deferasirox, deferiprone and deferoxamine. In some embodiments, the compound of the present disclosure, or the pharmaceutically acceptable salt thereof, or the pharmaceutical composition of the present disclosure is adminstered in an effective amount. In some embodiment, the additional therapeutic agent is adminstered in an effective amount.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the presently claimed subject matter as set forth in the claims. Furthermore, the following examples are illustrative, but not limiting, of the compounds, compositions and methods described herein. Other suitable modifications and adaptations known to those skilled in the art are within the scope of the following embodiments. Any and all journal articles, patent applications, issued patents, or other cited references are incorporated by reference in their entirety.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner.

In some embodiments, the following embodiments are provided:

1. A compound having a formula of Formula I, or a pharmaceutically acceptable salt thereof:

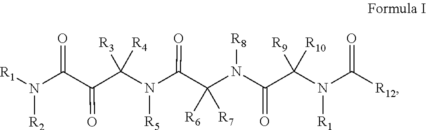

wherein:
R₁, R₂, R₃, R₄, R₅, R₆, R₇, R₈, R₉, R₁₀, R₁₁, and R₁₂ are each, independently, are each independently H, D, halo, alkyl, alkoxy, alkenyl, alkynyl, haloalkyl, haloalkoxy, aryl, cycloalkyl, heteroaryl, heterocycloalkyl, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)R^{a}NR^{a}R^{a}, S(O)NR^{a}R^{a}, S(O)₂R^{a}, SF₅, P(O)R^{a}R^{a}, P(O)(OR^{a})(OR^{a}), B(OR^{a})₂ and S(O)₂NR^{a}R^{a};
wherein when R₁, R₂, R₃, R₄, R₅, R₆, R₇, R₈, R₉, R₁₀, R₁₁, or R₁₂ is alkyl, alkoxy, alkenyl, alkynyl, haloalkyl, haloalkoxy, aryl, cycloalkyl, heteroaryl, heterocycloalkyl, then R₁, R₂, R₃, R₄, R₅, R₆, R₇, R₈, R₉, R₁₀, R₁₁, or R₁₂ is optionally substituted with at least one R^{b} substituent; or one or more selected from the group consisting of R₁ and R₂, R₁ and R₄, R₃ and R₄, R₆ and R₇, R₇ and R₈, and R₉ and R₁₀, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents;
each R^{a} is independently selected from H, D, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl;
wherein when R^{a} is C₁₋₆ alkyl, C₂₋₆ alkenyl, C₂₋₆ alkynyl, C₆₋₁₀ aryl, C₃₋₁₀cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a} is optionally substituted with 1, 2, 3, 4, or 5 independently selected R^{d} substituents; each R^{b} substituent is independently selected from a bond, H, D, halo, oxo, C₁₋₁₀ alkyl, C₁₋₆ alkoxy, C₁₋₄haloalkyl, C₁₋₄haloalkoxy, C₆₋₁₀ aryl, C₃₋₁₀cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR'S(O)R^{c}, NR'S(O)(═NR^{c})R^{c}, NR'S(O)₂R^{c}, NR'S(O)₂NR^{c}R^{c}, S(O)R^{c}, S(O)NR^{c}R^{c}, S(O)₂R^{c} or S(O)₂NR^{c}R^{c};
wherein when R^{b} is C₁₋₁₀alkyl, C₁₋₆alkoxy, C₁₋₄haloalkyl, C₁₋₄haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄alkyl- or (4-14 membered heterocycloalkyl)-C₁₋₄ alkyl, then R^{b} is optionally substituted with 1, 2, or 3 independently selected R^{d} substituents;
each R^{c} is independently selected from H, D, OH, C₁₋₆ alkyl, C₁₋₆ alkoxy, C₁₋₄haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl;
wherein when R^{c} is C₁₋₆alkyl, C₂₋₆alkenyl, C₂₋₆alkynyl, C₆₋₁₀ aryl, C₃₋₁₀cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents;
each R^{f} is independently selected from halogen, C₁₋₁₀alkyl, C₁₋₄haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R_{g}, C(O)OR^{g}, OC(O)R^{g}, OC(O)NR^{g}R_{g}, NHR^{g}, NR^{g}R_{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R_{g}, P(O)(OR^{g})(OR^{g}), S(O)R^{g}, S(O)NR^{g}R_{g}, S(O)₂R₉, NR^{g}S(O)₂R₉, NR^{g} S(O)₂NR^{g}R^{g}, and S(O)₂NR^{g}R^{g};
wherein when R^{f} is C₁₋₄alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents;
each Rⁿ is independently selected from C₁₋₁₀ alkyl, C₁₋₄haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, SF₅, P(O)R^{e}R^{e}, P(O)(OR^{e})(OR^{e}), S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e},
wherein when R^{d} is C₁₋₆alkyl, C₃₋₁₀cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents;
each R^{e} is independently selected from H, D, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{e} is C₁₋₆alkyl, C₂₋₆alkenyl, C₂₋₆alkynyl, C₆₋₁₀ aryl, C₃₋₁₀cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents;
each R^{g} is independently selected from H, D, C₁₋₆ alkyl, C₁₋₄haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents;
each R^{P} is independently selected from C₁₋₁₀ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r};
each R^{r} is independently selected from H, D, C₁₋₁₀ alkyl, C₃₋₆cycloalkyl, C₆₋₁₀ aryl, 5 or 6-membered heteroaryl, C₁₋₄haloalkyl, C₂₋₄alkenyl, and C₂₋₄ alkynyl, wherein when R^{r} is C₁₋₁₀ alkyl, C₃₋₆ cycloalkyl, C₆₋₁₀ aryl, 5 or 6-membered heteroaryl, C₂₋₄ alkenyl, and C₂₋₄ alkynyl, then R^{r} is optionally substituted with 1, 2 or 3 independently selected R^{q} substituents;
each R^{q} is independently selected from D, OH, CN, —COOH, NH₂, halo, C₁₋₆alkyl, C₁₋₆ haloalkyl, C₁₋₆ alkoxy, C₁₋₆ haloalkoxy, C₁₋₄ alkylthio, phenyl, 5-6 membered heteroaryl, C₃₋₆ cycloalkyl, 4-6 membered heterocycloalkyl, —CONHR^{t}, —NHC(O)R^{t}, —OC(O)R^{t}, —C(O)OR^{t}, —C(O)R^{t}, —SO₂R^{t}, —NHSO₂R^{t}, —SO₂NHR^{t} and NR^{t}R^{t},
wherein when R^{q} is C₁₋₆ alkyl, phenyl, 4-6 membered heterocycloalkyl or 5-6 membered heteroaryl, then R^{q} is optionally substituted with OH, CN, —COOH, NH₂, C₁₋₆ alkoxy, C₃. 6cycloalkyl or 4-6 membered heterocycloalkyl; and
each R^{t} is independently C₁₋₆ alkyl.

2. The compound of embodiment 1, where R₁, R₂, R₃, R₄, R₅, R₆, R₇, R₈, R₉, R₁₀, R₁₁, and R₁₂ are each, independently, H, D, OH, NH₂, NHC(═NH)NH₂, NHR^{a}, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl, wherein R^{a} is as defined in embodiment 1.

3. The compound of any one of embodiments 1-2, or a pharmaceutically acceptable salt thereof, wherein R₅ is H.

4. The compound of any one of embodiments 1-3, or a pharmaceutically acceptable salt thereof, wherein R₈ is H.

5. The compound of any one of embodiments 1-4, or a pharmaceutically acceptable salt thereof, wherein R₁₁ is H.

6. The compound of any one of embodiments 1-5, or a pharmaceutically acceptable salt thereof, wherein R₃ is H.

7. The compound of any one of embodiments 1-6, or a pharmaceutically acceptable salt thereof, wherein R₆ is H.

8. The compound of any one of embodiments 1-7, or a pharmaceutically acceptable salt thereof, wherein R₉ is H.

9. The compound of embodiment 1, or a pharmaceutically acceptable salt thereof, wherein the compound having a formula of

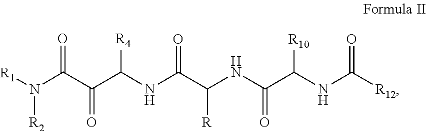

wherein the variables are as defined in embodiments 1-2.

10. The compound of embodiment 1, or a pharmaceutically acceptable salt thereof, wherein the compound has a formula of

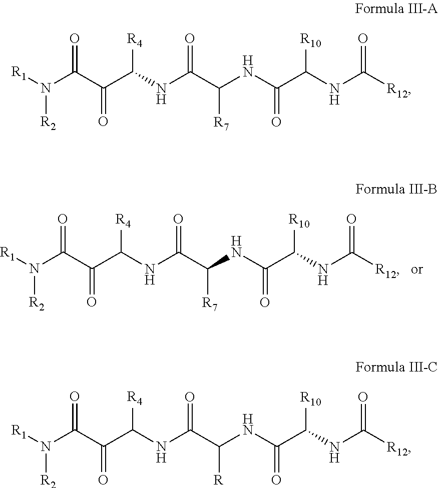

wherein the variables are as defined in embodiments 1-2.

11. The compound of embodiment 1, or a pharmaceutically acceptable salt thereof, wherein the compound has a formula of

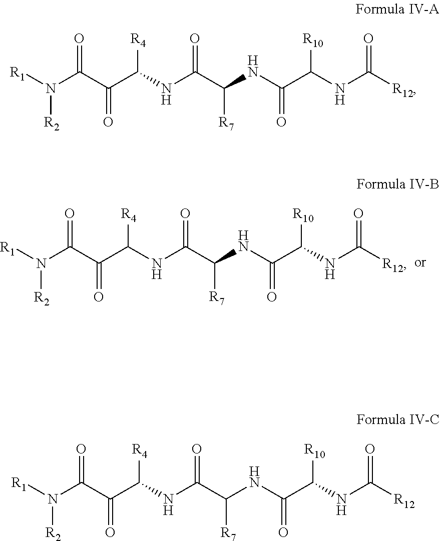

wherein the variables are as defined in embodiments 1-2.

12. The compound of embodiment 1, or a pharmaceutically acceptable salt thereof, wherein the compound has a formula of

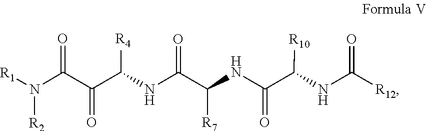

wherein the variables are as defined in embodiments 1-2.

13. The compound of any one of embodiments 1-12, or a pharmaceutically acceptable salt thereof, wherein R₁ is optionally substituted C₁-C₆ alkyl.

14. The compound of embodiment 13, or a pharmaceutically acceptable salt thereof, wherein R₁ is Me.

15. The compound of any one of embodiments 1-12, or a pharmaceutically acceptable salt thereof, wherein R₁ is H.

16. The compound of any one of embodiments 1-15, or a pharmaceutically acceptable salt thereof, wherein R₂ is H, Me, Et, t-Bu, —CH₂CN, -3-pyridyl, —CH₂C(O)OR₁₇,

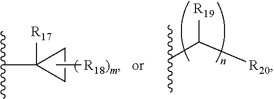

wherein:
R₁₇, R₁₈, R₁₉, and R₂₀ are each, independently, H, OH, NH₂, optionally substituted C₁-C₆ alkyl, optionally substituted C₁-C₆ hydroxyalkyl, optionally substituted C₁-C₆ alkoxy, optionally substituted cycloalkyl, optionally substituted pyridyl, optionally substituted heteroaryl, optionally substituted aryl, or optionally substituted cycloheteroalkyl;
m is 0-4; and
n is 0-10.

17. The compound of embodiment 16, or a pharmaceutically acceptable salt thereof, wherein R₂ is Me.

18. The compound of embodiment 16, or a pharmaceutically acceptable salt thereof, wherein R₂ is Et.

19. The compound of any one of embodiments 16-18, or a pharmaceutically acceptable salt thereof, wherein R₂ is 3-pyridyl.

20. The compound of any one of embodiments 16-18, or a pharmaceutically acceptable salt thereof, wherein R₁₇ is Me.

21. The compound of any one of embodiments 16-20, or a pharmaceutically acceptable salt thereof, wherein R₁₈ is halo.

22. The compound of embodiment 20, or a pharmaceutically acceptable salt thereof, wherein R₁₈ is F.

23. The compound of and one of embodiments 16-22, or a pharmaceutically acceptable salt thereof, wherein m is 1.

24. The compound of embodiment 23, or a pharmaceutically acceptable salt thereof, wherein R₁₉ is H.

25. The compound of any one of embodiments 19-24, or a pharmaceutically acceptable salt thereof, wherein n is 1.

26. The compound of embodiment 25, or a pharmaceutically acceptable salt thereof, wherein R₂₀ is 3-pyridyl, —C(O)OR₂₁, or CN, wherein R₂₁. is optionally substituted C₁-C₆alkyl, optionally substituted C₁-C₆ hydroxyalkyl, optionally substituted C₁-C₆ alkoxy, optionally substituted cycloalkyl, or optionally substituted cycloheteroalkyl.

27. The compound of embodiment 26, or a pharmaceutically acceptable salt thereof, wherein R₂₁. is optionally substituted C₁-C₆ alkyl.

28. The compound of embodiment 26, or a pharmaceutically acceptable salt thereof, wherein R₂₁ is ethyl.

29. The compound of embodiment 1, or a pharmaceutically acceptable salt thereof, wherein R₂ and R₄ together with the atom to which they are attached, form a 3-, 4-, 5-, 6-, or 7-membered cycloalkyl or heterocycloalkyl ring optionally substituted with 1, 2, 3, 4 or 5 independently selected R^{b} substituents, wherein R^{b} is as defined in embodiment 1.

30. The compound of embodiment 29, or a pharmaceutically acceptable salt thereof, wherein R₁ and R₄ together with the atom to which they are attached, form a 7-membered heterocycloalkyl ring optionally substituted with 1, 2, 3, 4, or 5 independently selected R^{b} substituents, wherein R^{b} is as defined in embodiment 1.

31. The compound of any one of embodiments 1-28, or a pharmaceutically acceptable salt thereof, wherein R₄ is

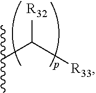

wherein R₃₂ and R₃₃ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; and p is 0-10.

32. The compound embodiment 31, or a pharmaceutically acceptable salt thereof, wherein R₃₂ is H.

33. The compound of any one of embodiments 31-32, or a pharmaceutically acceptable salt thereof, wherein p is 1.

34. The compound of any one of embodiments 31-32, or a pharmaceutically acceptable salt thereof, wherein p is 2.

35. The compound of any one of embodiments 31-32, or a pharmaceutically acceptable salt thereof, wherein p is 3.

36. The compound of any one of embodiments 31-35, or a pharmaceutically acceptable salt thereof, wherein R₃₃ is H,

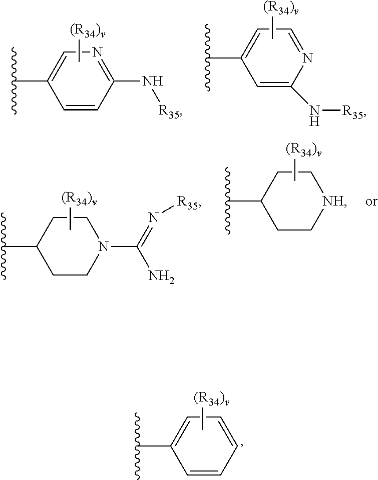

wherein R₃₄ and R₃₅ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl and v is 0-5.

37. The compound embodiment 36, or a pharmaceutically acceptable salt thereof, wherein R₃₄ is H.

38. The compound of any one of embodiments 36-37, or a pharmaceutically acceptable salt thereof, wherein v is 0.

39. The compound of any one of embodiments 36-38, or a pharmaceutically acceptable salt thereof, wherein v is 1.

40. The compound of any one of embodiments 36-39, or a pharmaceutically acceptable salt thereof, wherein R₃₅ is H.

41. The compound of any one of embodiments 31-35, or a pharmaceutically acceptable salt thereof, wherein R₃₃ is

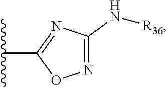

wherein R₃₆ is H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

42. The compound embodiment 41, or a pharmaceutically acceptable salt thereof, wherein R₃₆ is H.

43. The compound of any one of embodiments 31-35, or a pharmaceutically acceptable salt thereof, wherein R₃₃ is

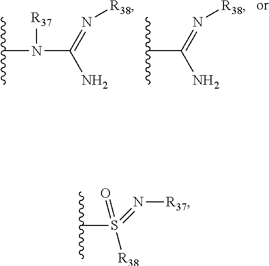

wherein R₃₇ and R₃₈ are each, independently, H, D, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl.

44. The compound of embodiment 43, or a pharmaceutically acceptable salt thereof, wherein R₃₇ is H.

45. The compound of any one of embodiments 43-44, or a pharmaceutically acceptable salt thereof, wherein R₃₈ is H or NH₂.

46. The compound of any one of embodiments 43-44, or a pharmaceutically acceptable salt thereof, wherein R₃₈ is optionally substituted C₁-C₆ alkyl.

47. The compound of any one of embodiments 43-44, or a pharmaceutically acceptable salt thereof, wherein R₃₈ is Me.

48. The compound of any one of embodiments 43-44, or a pharmaceutically acceptable salt thereof, wherein R₃₈ is optionally substituted C₁-C₆ alkoxy or NH₂.

49. The compound of any one of embodiments 43-44, or a pharmaceutically acceptable salt thereof, wherein R₃₈ is MeO.

50. The compound of any one of embodiments 1-49, or a pharmaceutically acceptable salt thereof, wherein R₇ is

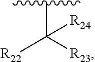

wherein R₂₂, R₂₃, and R₂₄, are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₂₂ and R₂₃ together with the atom to which they are attached, form an optionally substituted cycloalkyl or optionally substituted cycloheteroalkyl.

51. The compound of embodiment 50, or a pharmaceutically acceptable salt thereof, wherein R₇ is

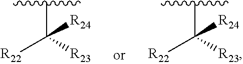

wherein R₂₂, R₂₃, and R₂₄ are as defined in embodiment 50.

52. The compound of embodiment 51, or a pharmaceutically acceptable salt thereof, wherein R₂₂ is Me.

53. The compound of embodiment 52, or a pharmaceutically acceptable salt thereof, wherein R₂₃ is OH, OMe, Me, or Et.

54. The compound of embodiment 51, or a pharmaceutically acceptable salt thereof, wherein R₂₂ and R₂₃ together with the atom to which they are attached, form an optionally substituted cyclobutane.

55. The compound of embodiment 51, or a pharmaceutically acceptable salt thereof, wherein R₂₂ and R₂₃ together with the atom to which they are attached, form an optionally substituted cyclopetane or cyclohexane.

56. The compound of any one of embodiments 51-55, or a pharmaceutically acceptable salt thereof, wherein R₂₄ is Me.

57. The compound of any one of embodiments 51-55, or a pharmaceutically acceptable salt thereof, wherein R₂₄ is H.

58. The compound of embodiment 1, or a pharmaceutically acceptable salt thereof, wherein R₇ and R₈ are together with the atoms to which they are attached, form a 3-, 4-, 5-, 6-, or 7-membered cycloalkyl or heterocycloalkyl ring optionally substituted with 1, 2, 3, 4 or 5 independently selected R^{b} substituents, wherein R^{b} is as defined in embodiment 1.

59. The compound of embodiment 58, or a pharmaceutically acceptable salt thereof, wherein R₇ and R₈ together are

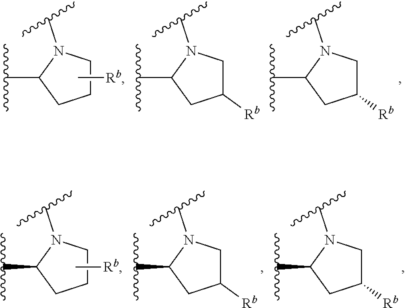

wherein R^{b} is as defined in embodiment 1.

60. The compound of any one of embodiments 58-59, or a pharmaceutically acceptable salt thereof, wherein R^{b} is H.

61. The compound of any one of embodiments 58-59, or a pharmaceutically acceptable salt thereof, wherein R^{b} is OH or CN.

62. The compound of any one of embodiments 1-61, or a pharmaceutically acceptable salt thereof, R₁₀ is

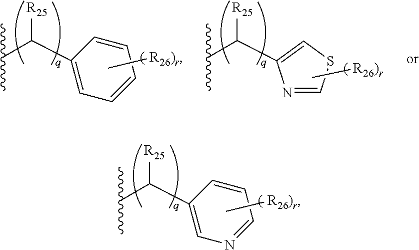

wherein:
R₂₅ and R₂₆, are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl;
q is 0-10; and
r is 0-5.

63. The compound of embodiment 62, or a pharmaceutically acceptable salt thereof, wherein R₂₅ is H.

64. The compound of any one of embodiments 62-63, or a pharmaceutically acceptable salt thereof, wherein q is 1.

65. The compound of any one of embodiments 62-63, or a pharmaceutically acceptable salt thereof, wherein q is 2, 3, or 4.

66. The compound of any one of embodiments 62-65, or a pharmaceutically acceptable salt thereof, wherein r is 0.

67. The compound of any one of embodiments 62-65, or a pharmaceutically acceptable salt thereof, wherein r is 1.

68. The compound of any one of embodiments 62-67, or a pharmaceutically acceptable salt thereof, wherein R₂₆ is Me.

69. The compound of any one of embodiments 62-67, or a pharmaceutically acceptable salt thereof, wherein R₂₆ is halo.

70. The compound of embodiment 69, or a pharmaceutically acceptable salt thereof, wherein R₂₆ is F.

71. The compound of any one of embodiments 1-70, or a pharmaceutically acceptable salt thereof, wherein R₁₂ is

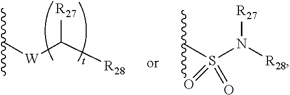

wherein:
W is O, S, NR₂₉, S(O)₂NR₂₉, S(O)₂, S(O)₂NH, or CR₃₀R₃₁;
R₂₇, R₂₈, R₂₉, R₃₀ and R₃₁ are each, independently, H, D, optionally substituted C₁-C₆ alkyl, optionally substituted C₁-C₆ hydroxyalkyl, optionally substituted C₁-C₆ alkoxy, optionally substituted cycloalkyl, optionally substituted aryl, optionally substituted heteroaryl, or optionally substituted cycloheteroalkyl; and
t is 0-10.

72. The compound of embodiment 71, or a pharmaceutically acceptable salt thereof, wherein W is O.

73. The compound of embodiment 71, or a pharmaceutically acceptable salt thereof, wherein W is CR₃₀R₃₁.

74. The compound of embodiment 73, or a pharmaceutically acceptable salt thereof, wherein W is CH₂.

75. The compound of embodiment 71, or a pharmaceutically acceptable salt thereof, wherein W is NR₂₉,

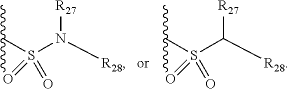

76. The compound of embodiment 73, or a pharmaceutically acceptable salt thereof, wherein R₂₉ is H.

77. The compound of any one of embodiments 71-76, or a pharmaceutically acceptable salt thereof, wherein R₂₇ is H.

78. The compound of any one of embodiments 71-77, or a pharmaceutically acceptable salt thereof, wherein t is 1-4.

79. The compound of any one of embodiments 71-78, or a pharmaceutically acceptable salt thereof, wherein R₂₈ is methyl, ethyl, cyclopropyl, or phenyl.

80. The compound of any one of embodiments 71-78, or a pharmaceutically acceptable salt thereof, wherein R₂₈ is optionally substituted C₁-C₆ alkyl.

81. The compound of embodiment 80, or a pharmaceutically acceptable salt thereof, wherein R₂₈ is Me

82. The compound of embodiment 80, or a pharmaceutically acceptable salt thereof, wherein R₂₈ is Et.

83. The compound of embodiment 80, or a pharmaceutically acceptable salt thereof, wherein R₂₈ is isopropyl.

84. A compound or a pharmaceutically acceptable salt thereof, wherein the compound is a compound as described herein.

85. A compound having a formula of:

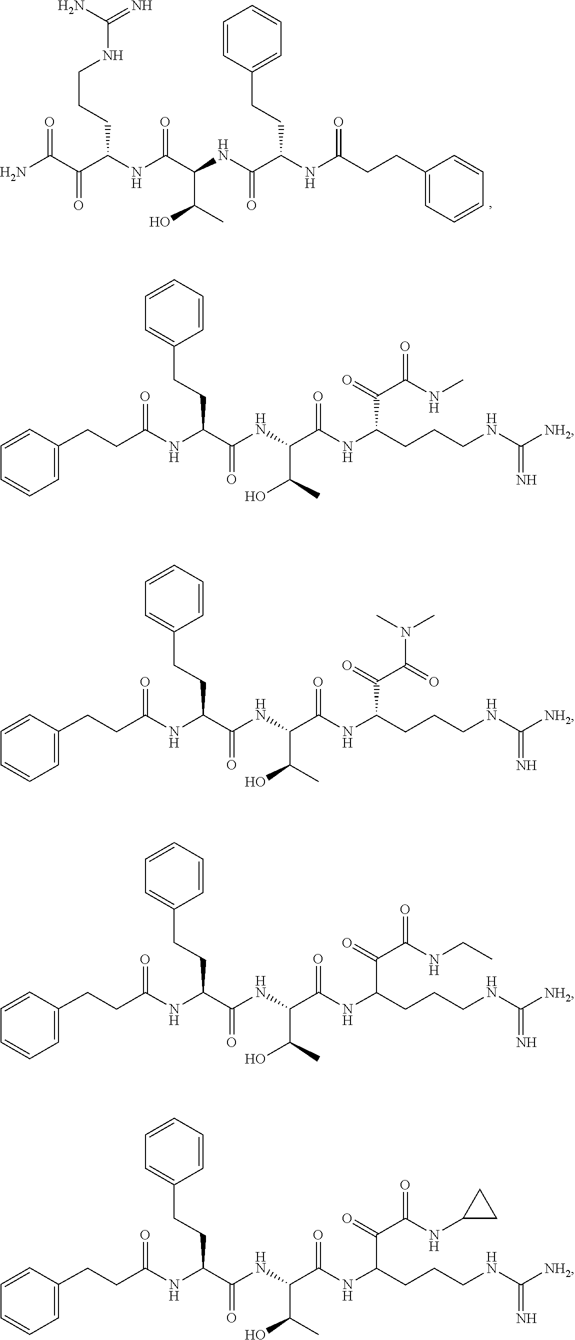 [SMILES: C[C@@H](O)[C@H](NC(=O)[C@H](CCc1ccccc1)NC(=O)CCc1ccccc1)C(=O)N[C@@H](CCCNC(=N)N)C(=O)C(N)=O ; CNC(=O)C(=O)[C@H](CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)N[C@@H](CCCNC(=N)N)C(=O)C(=O)N(C)C ; CCNC(=O)C(=O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NC1CC1]

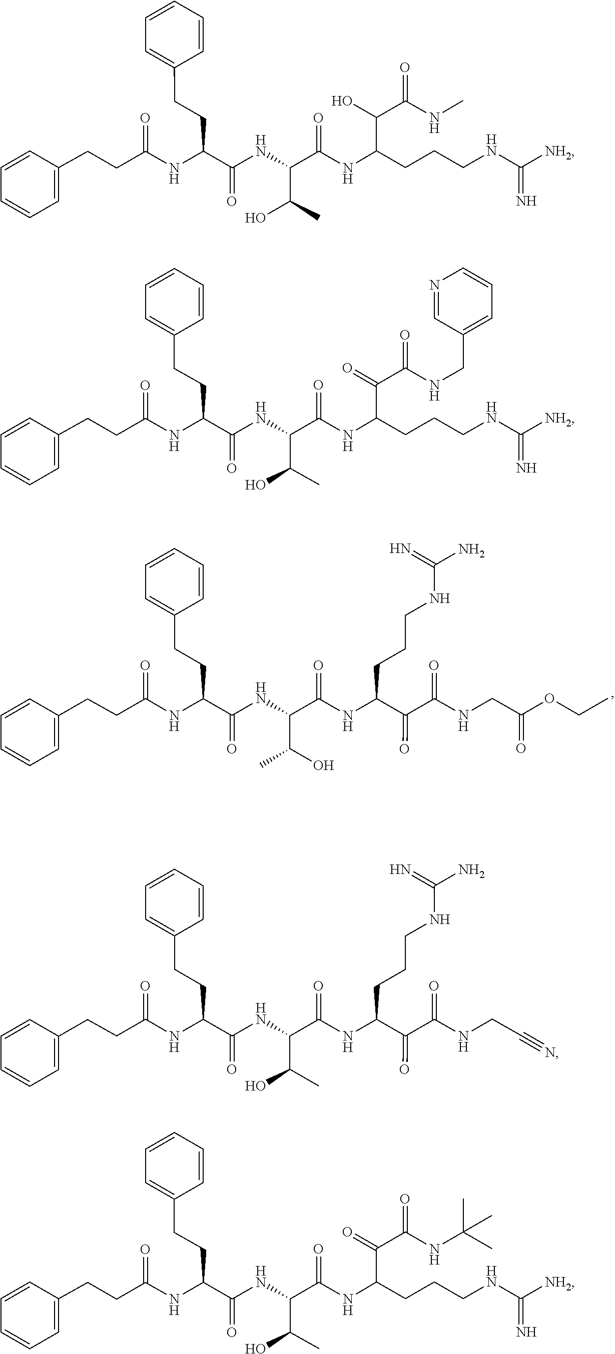 [SMILES: CNC(=O)C(O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NCc1cccnc1 ; CCOC(=O)CNC(=O)C(=O)[C@H](CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)N[C@@H](CCCNC(=N)N)C(=O)C(=O)NCC#N ; C[C@@H](O)[C@H](NC(=O)[C@H](CCc1ccccc1)NC(=O)CCc1ccccc1)C(=O)NC(CCCNC(=N)N)C(=O)C(=O)NC(C)(C)C]

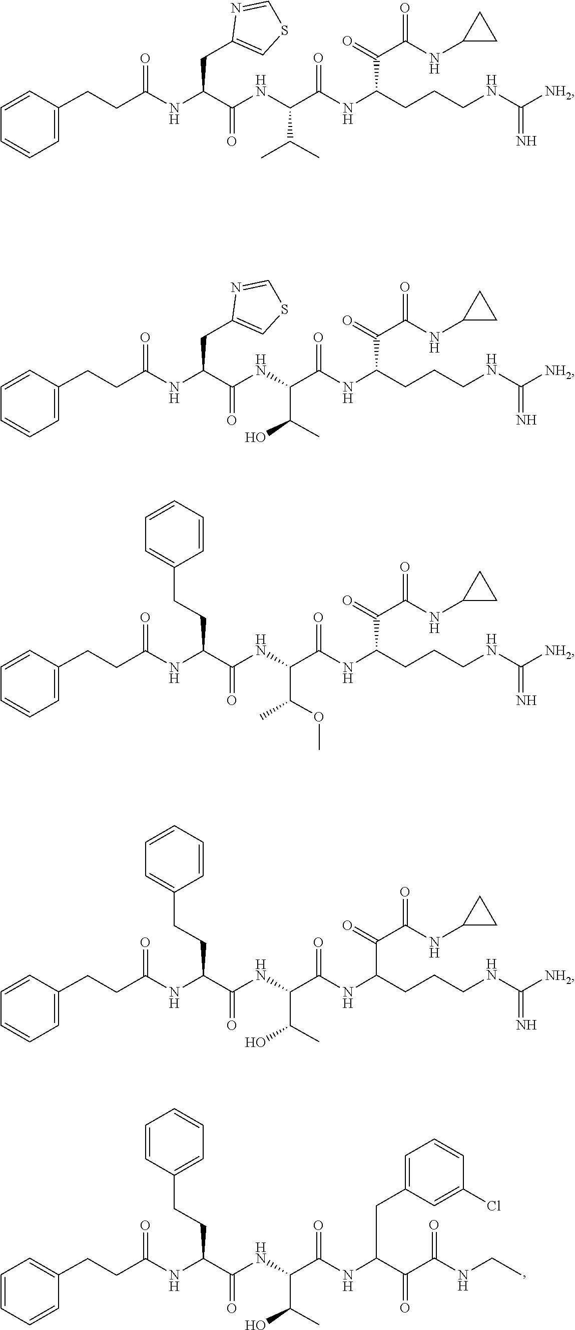 [SMILES: CC(C)[C@H](NC(=O)[C@H](Cc1cscn1)NC(=O)CCc1ccccc1)C(=O)N[C@@H](CCCNC(=N)N)C(=O)C(=O)NC1CC1 ; C[C@@H](O)[C@H](NC(=O)[C@H](Cc1cscn1)NC(=O)CCc1ccccc1)C(=O)N[C@@H](CCCNC(=N)N)C(=O)C(=O)NC1CC1 ; CO[C@H](C)[C@H](NC(=O)[C@H](CCc1ccccc1)NC(=O)CCc1ccccc1)C(=O)N[C@@H](CCCNC(=N)N)C(=O)C(=O)NC1CC1 ; C[C@H](O)[C@H](NC(=O)[C@H](CCc1ccccc1)NC(=O)CCc1ccccc1)C(=O)NC(CCCNC(=N)N)C(=O)C(=O)NC1CC1 ; CCNC(=O)C(=O)C(Cc1cccc(Cl)c1)NC(=O)[C@@H](NC(=O)[C@H](CCc1ccccc1)NC(=O)CCc1ccccc1)[C@@H](C)O]

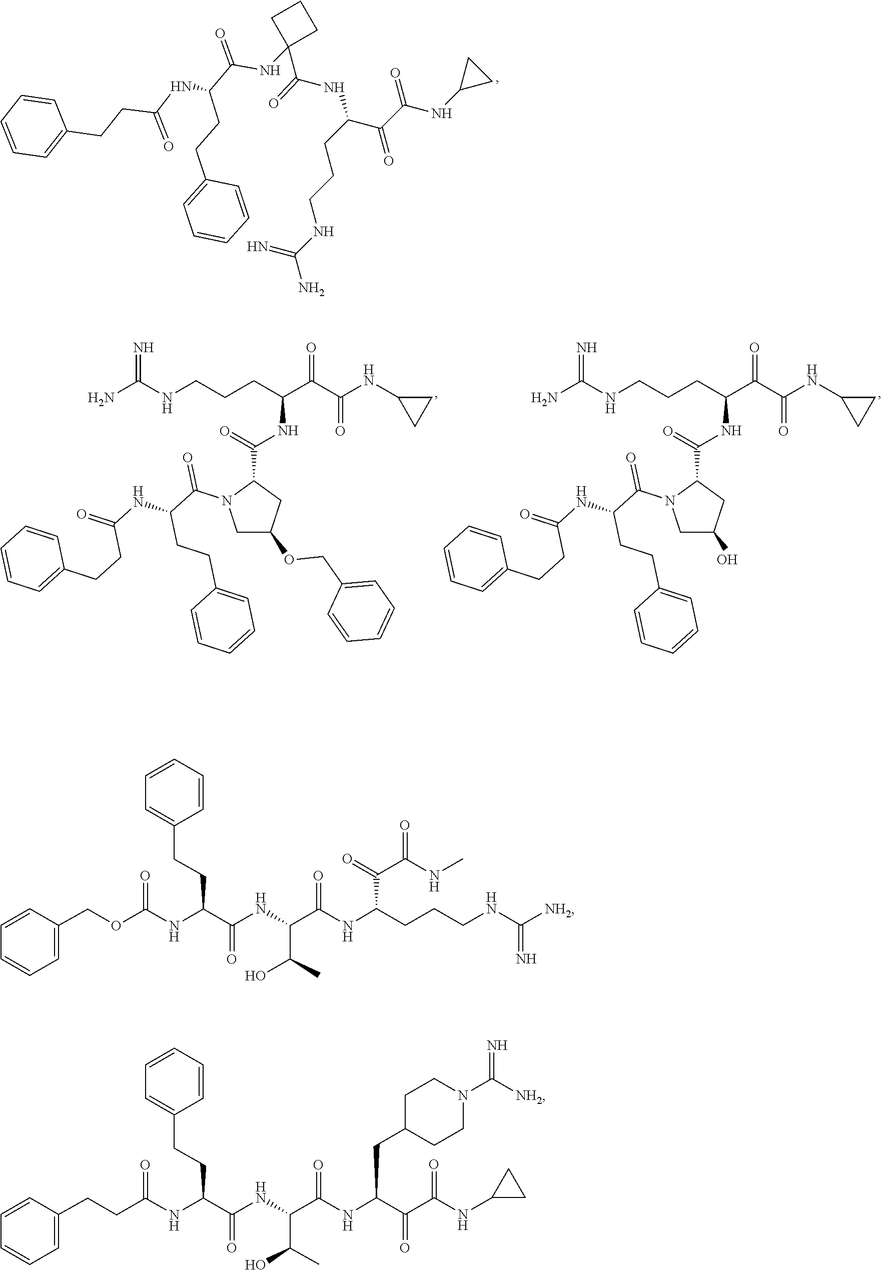 [SMILES: N=C(N)NCCC[C@H](NC(=O)C1(NC(=O)[C@H](CCc2ccccc2)NC(=O)CCc2ccccc2)CCC1)C(=O)C(=O)NC1CC1 ; N=C(N)NCCC[C@H](NC(=O)[C@@H]1C[C@@H](OCc2ccccc2)CN1C(=O)[C@H](CCc1ccccc1)NC(=O)CCc1ccccc1)C(=O)C(=O)NC1CC1 ; N=C(N)NCCC[C@H](NC(=O)[C@@H]1C[C@@H](O)CN1C(=O)[C@H](CCc1ccccc1)NC(=O)CCc1ccccc1)C(=O)C(=O)NC1CC1 ; CNC(=O)C(=O)[C@H](CCCNC(=N)N)NC(=O)[C@@H](NC(=O)[C@H](CCc1ccccc1)NC(=O)OCc1ccccc1)[C@@H](C)O ; C[C@@H](O)[C@H](NC(=O)[C@H](CCc1ccccc1)NC(=O)CCc1ccccc1)C(=O)N[C@@H](CC1CCN(C(=N)N)CC1)C(=O)C(=O)NC1CC1]

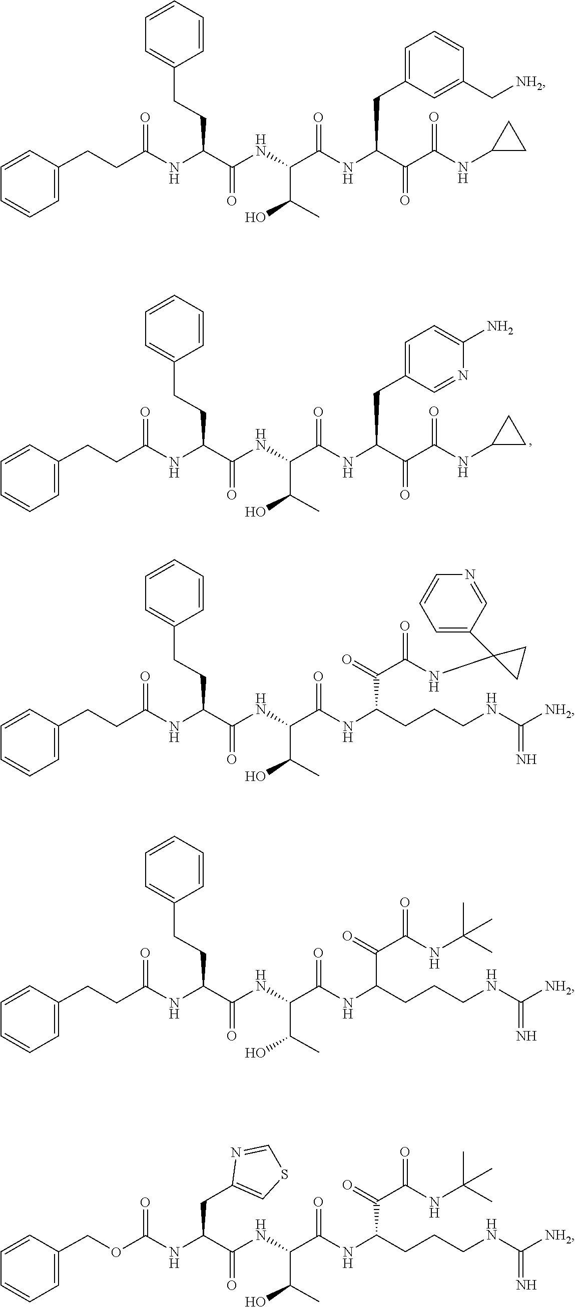

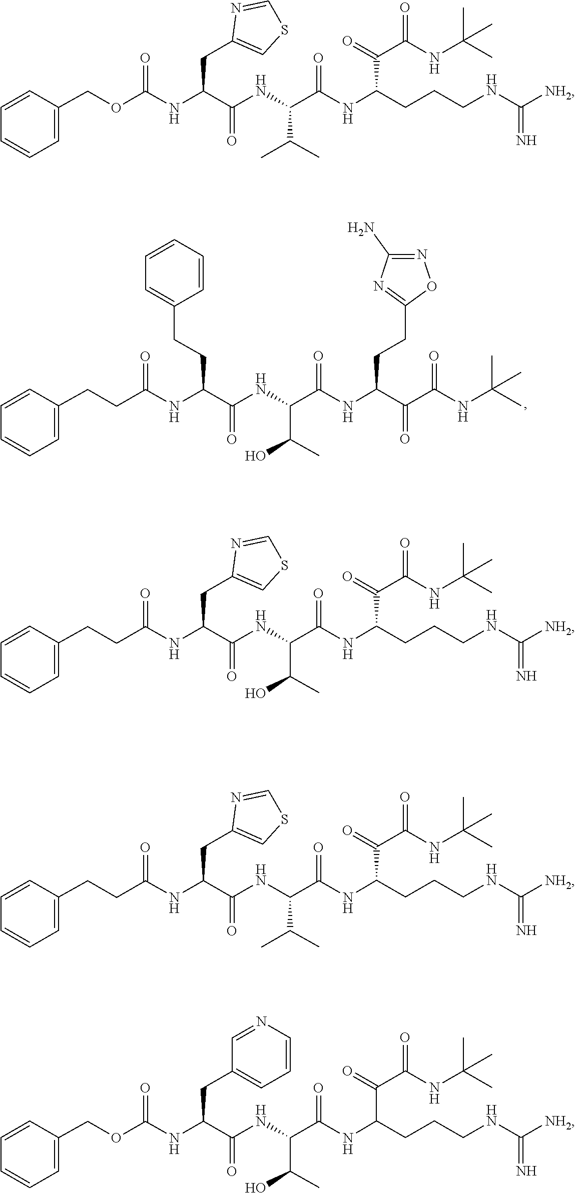 [SMILES: CC(C)[C@H](NC(=O)[C@H](Cc1cscn1)NC(=O)OCc1ccccc1)C(=O)N[C@@H](CCCNC(=N)N)C(=O)C(=O)NC(C)(C)C ; C[C@@H](O)[C@H](NC(=O)[C@H](CCc1ccccc1)NC(=O)CCc1ccccc1)C(=O)N[C@@H](CCc1nc(N)no1)C(=O)C(=O)NC(C)(C)C ; C[C@@H](O)[C@H](NC(=O)[C@H](Cc1cscn1)NC(=O)CCc1ccccc1)C(=O)N[C@@H](CCCNC(=N)N)C(=O)C(=O)NC(C)(C)C ; CC(C)[C@H](NC(=O)[C@H](Cc1cscn1)NC(=O)CCc1ccccc1)C(=O)N[C@@H](CCCNC(=N)N)C(=O)C(=O)NC(C)(C)C ; C[C@@H](O)[C@H](NC(=O)[C@H](Cc1cccnc1)NC(=O)OCc1ccccc1)C(=O)NC(CCCNC(=N)N)C(=O)C(=O)NC(C)(C)C]

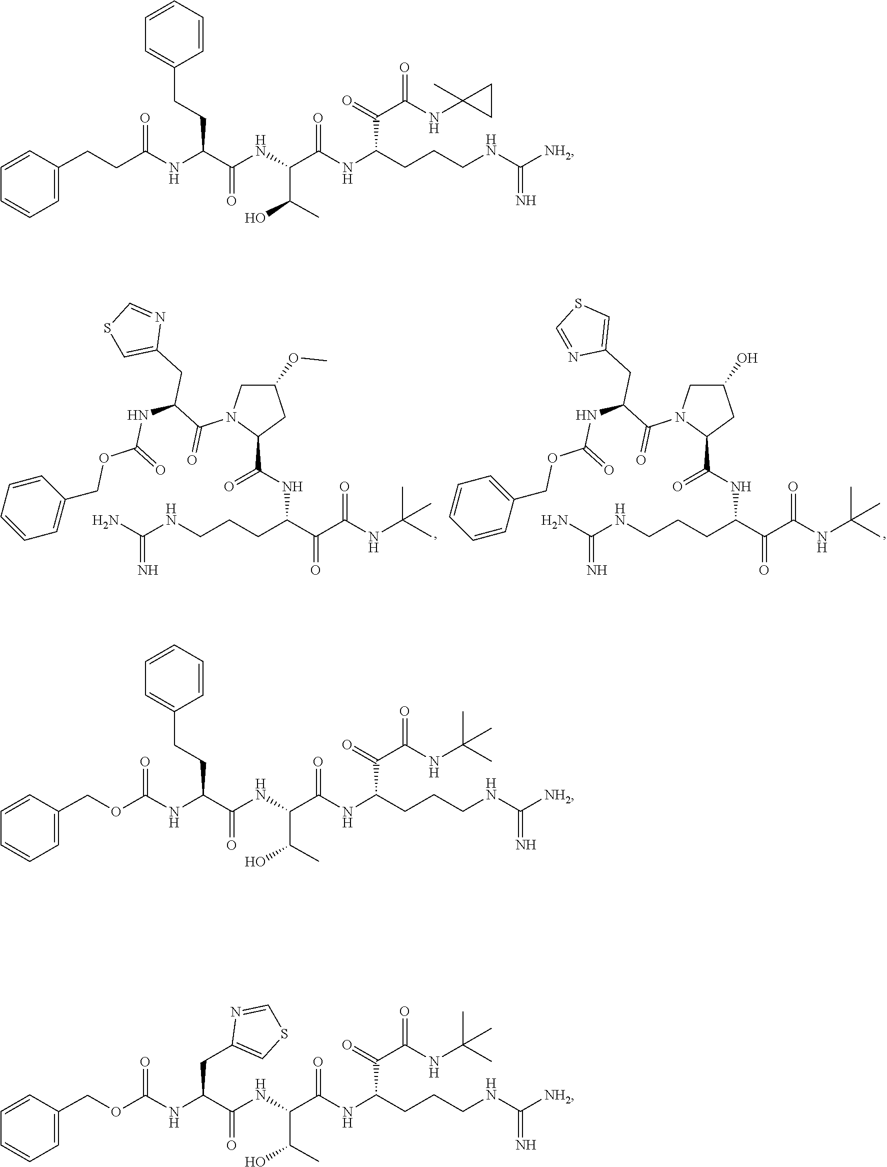

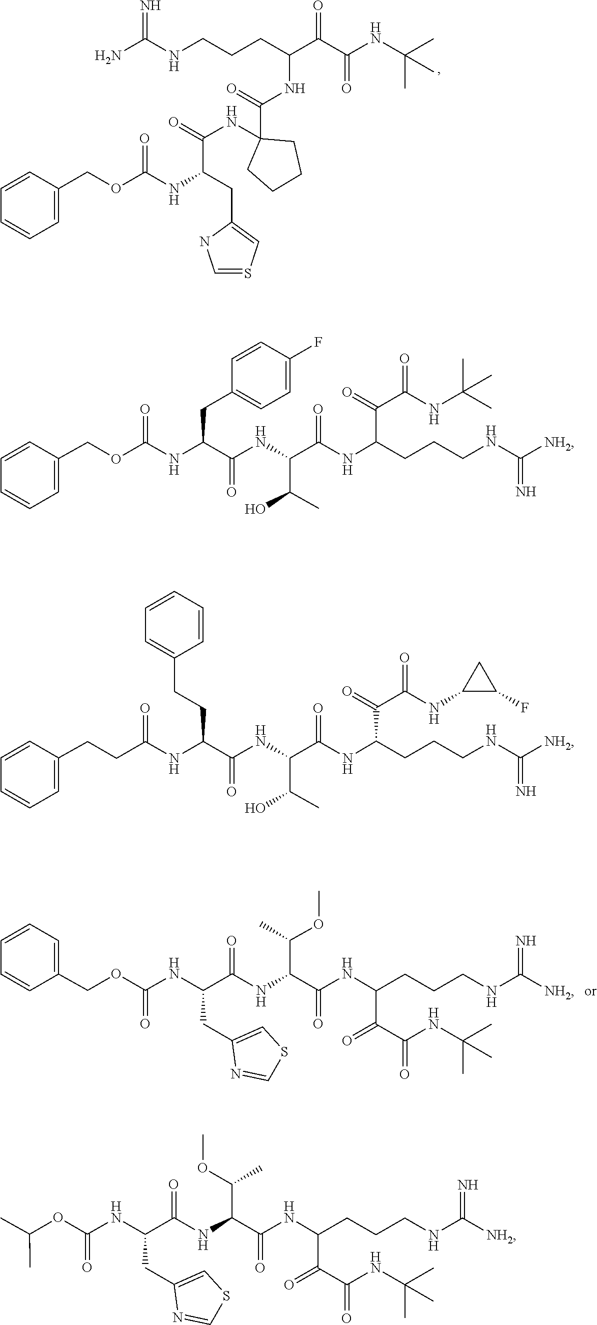

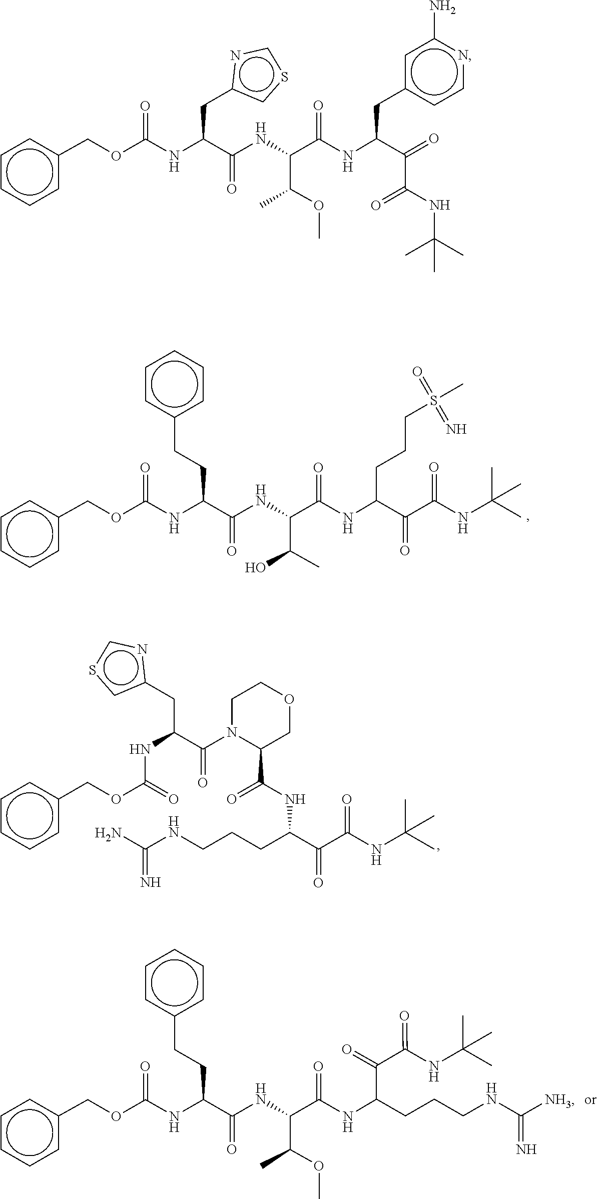

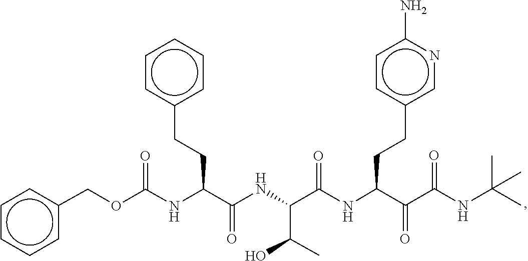

or a pharmaceutically acceptable salt thereof.

86. A pharmaceutical composition comprising a compound of any one of embodiments 1-85, or a pharmaceutically acceptable salt thereof.

87. A pharmaceutical composition comprising the compound of any one of embodiments 1-85, or a pharmaceutically acceptable salt thereof, and a pharmaceutically acceptable carrier, adjuvant, or vehicle.

88. A method for treating a low hepcidin disorder, disease, and/or condition in a subject, comprising administering to the subject the compound of any one of embodiments 1-85, or a pharmaceutically acceptable salt thereof, or the pharmaceutical composition of any one of embodiments 86-87.

89. A method for increasing hepcidin production by the liver in a subject, comprising administering to the subject the compound of any one of embodiments 1-85, or a pharmaceutically acceptable salt thereof, or the pharmaceutical composition of any one of embodiments 86-87.

90. A method for treating an iron overload disorder, disease, and/or condition in a subject, comprising administering to the subject the compound of any one of embodiments 1-85, or a pharmaceutically acceptable salt thereof, or the pharmaceutical composition of any one of embodiments 86-87.

91. The method of embodiment 90, wherein the iron overload disorder, disease, and/or condition is selected from the group consisting of hemochromatosis Type 1, 2a, 2b and 3 (hemochromatosis, Hfe hemochromatosis (Type 1), juvenile hemochromatosis (types 2a and 2b),), hepcidin deficiency, transfusional iron overload, African iron overload, and iron overload cardiomyopathy.

92. A method for treating an iron loading anemia in a subject, comprising administering to the subject the compound of any one of embodiments 1-85, or a pharmaceutically acceptable salt thereof, or the pharmaceutical composition of any one of embodiments 86-87.

93. The method of embodiment 92, wherein the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia, congenital dyserythropoietic anemias (Type I and Type II), pyruvate kinase deficiency, and myelodysplasia (such as myelodysplastic syndrome, and RARS SF3B1 associated MDS).

94. A method for treating a hematological disease, disorder, and/or condition in a subject, comprising administering to the subject the compound of any one of embodiments 1-85, or a pharmaceutically acceptable salt thereof, or the pharmaceutical composition of any one of embodiments 86-87.

95. The method of embodiment 94, wherein the hematological disease, disorder, and/or condition is selected from the group consisting of sickle cell disease (such as sickle cell anemia), polycythemia vera, sideroblastic anemia, and bone marrow transplantation.

96. A method for treating a liver disease in a subject, comprising administering to the subject the compound of any one of embodiments 1-85, or a pharmaceutically acceptable salt thereof, or the pharmaceutical composition of any one of embodiments 86-87.

97. The method of embodiment 96, wherein the liver disease is selected from the group consisting of Hepatitis B, Hepatitis C, alcoholic liver disease, cirrhosis of the liver, hepatocellular carcinoma, and non-alcoholic steatohepatitis (NASH).

98. A method of treating a metabolic disease in a subject, comprising administering to the subject the compound of any one of embodiments 1-85, or a pharmaceutically acceptable salt thereof, or the pharmaceutical composition of any one of embodiments 86-87.

99. The method of embodiment 98, wherein the metabolic disease is selected from the group consisting of metabolic syndrome, insulin resistance, Type II diabetes, porphyria, porphyria cutanea tarda, Wilson's Disease, and acute iron overdose.

100. A method for treating a neurodegenerative disorder in a subject, comprising administering to the subject the compound of any one of embodiments 1-85, or a pharmaceutically acceptable salt thereof, or the pharmaceutical composition of any one of embodiments 86-87.

101. A method for treating an infectious disease in a subject, comprising administering to the subject the compound of any one of embodiments 1-85, or a pharmaceutically acceptable salt thereof, or the pharmaceutical composition of any one of embodiments 86-87.

102. The method of embodiment 101, wherein the infectious disease is a siderophilic infection.

103. The method of any one of embodiments 88-102, wherein the subject is a subject in need thereof.

104. A method of inhibiting matriptase 2, or a mutant thereof, in a biological sample, comprising contacting the sample with the compound of any one of embodiments 1-85, or a pharmaceutically acceptable salt thereof, or the pharmaceutical composition of any one of embodiments 86-87.

105. The method of any one of embodiments 88-104, wherein the compound of any one of embodiments 1-85, or a pharmaceutically acceptable salt thereof, or the pharmaceutical composition of any one of embodiments 86-87 is administered in an effective amount.

106. Use of a compound of any one of embodiments 1-85, or a pharmaceutically acceptable salt thereof, or the pharmaceutical composition of any one of embodiments 86-87 for treating a low hepcidin disorder, disease, and/or condition in a subject.

107. Use of a compound of any one of embodiments 1-85, or a pharmaceutically acceptable salt thereof, or the pharmaceutical composition of any one of embodiments 86-87 in the manufacture of a medicament for treating a low hepcidin disorder, disease, and/or condition in a subject.

108. A compound of any one of embodiments 1-85, or a pharmaceutically acceptable salt thereof, or the pharmaceutical composition of any one of embodiments 86-87 in use for treating a low hepcidin disorder, disease, and/or condition in a subject.

109. Use of a compound of any one of embodiments 1-85, or a pharmaceutically acceptable salt thereof, or the pharmaceutical composition of any one of embodiments 86-87 for increasing hepcidin production in a subject.

110. Use of a compound of any one of embodiments 1-85, or a pharmaceutically acceptable salt thereof, or the pharmaceutical composition of any one of embodiments 86-87 in the manufacture of a medicament for increasing hepcidin production in a subject.

111. A compound of any one of embodiments 1-85, or a pharmaceutically acceptable salt thereof, or the pharmaceutical composition of any one of embodiments 86-87 in use for increasing hepcidin production in a subject.

112. Use of a compound of any one of embodiments 1-85, or a pharmaceutically acceptable salt thereof, or the pharmaceutical composition of any one of embodiments 86-87 for treating an iron overload disorder, disease, and/or condition in a subject.

113. Use of a compound of any one of embodiments 1-85, or a pharmaceutically acceptable salt thereof, or the pharmaceutical composition of any one of embodiments 86-87 in the manufacture of a medicament for treating an iron overload disorder, disease, and/or condition in a subject.

114. A compound of any one of embodiments 1-85, or a pharmaceutically acceptable salt thereof, or the pharmaceutical composition of any one of embodiments 86-87 in use for treating an iron overload disorder, disease, and/or condition in a subject.

115. The use of any one of embodiments 112-114, wherein the iron overload disorder, disease, and/or condition is selected from the group consisting of hemochromatosis Type 1, 2a, 2b and 3 (hemochromatosis, Hfe hemochromatosis (Type 1), juvenile hemochromatosis (types 2a and 2b),), hepcidin deficiency, transfusional iron overload, African iron overload, and iron overload cardiomyopathy.

116. Use of a compound of any one of embodiments 1-85, or a pharmaceutically acceptable salt thereof, or the pharmaceutical composition of any one of embodiments 86-87 for treating an iron loading anemia in a subject.

117. Use of a compound of any one of embodiments 1-85, or a pharmaceutically acceptable salt thereof, or the pharmaceutical composition of any one of embodiments 86-87 in the manufacture of a medicament for treating an iron loading anemia in a subject.

118. A compound of any one of embodiments 1-85, or a pharmaceutically acceptable salt thereof, or the pharmaceutical composition of any one of embodiments 86-87 in use for treating an iron loading anemia in a subject.

119. The use of any one of embodiments 116-118, wherein the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia, congenital dyserythropoietic anemias (Type I and Type II), pyruvate kinase deficiency, and myelodysplasia (such as myelodysplastic syndrome, and RARS SF3B1 associated MDS).

120. Use of a compound of any one of embodiments 1-85, or a pharmaceutically acceptable salt thereof, or the pharmaceutical composition of any one of embodiments 86-87 for treating a hematological disease, disorder, and/or condition in a subject.

121. Use of a compound of any one of embodiments 1-85, or a pharmaceutically acceptable salt thereof, or the pharmaceutical composition of any one of embodiments 86-87 in the manufacture of a medicament for treating a hematological disease, disorder, and/or condition in a subject.

122. A compound of any one of embodiments 1-85, or a pharmaceutically acceptable salt thereof, or the pharmaceutical composition of any one of embodiments 86-87 in use for treating a hematological disease, disorder, and/or condition in a subject.

123. The use of any one of embodiments 120-122, wherein the hematological disease, disorder, and/or condition is selected from the group consisting of sickle cell disease (such as sickle cell anemia), polycythemia vera, sideroblastic anemia, and bone marrow transplantation.

124. Use of a compound of any one of embodiments 1-85, or a pharmaceutically acceptable salt thereof, or the pharmaceutical composition of any one of embodiments 86-87 for treating a liver disease in a subject in a subject.

125. Use of a compound of any one of embodiments 1-85, or a pharmaceutically acceptable salt thereof, or the pharmaceutical composition of any one of embodiments 86-87 in the manufacture of a medicament for treating a liver disease in a subject.

126. A compound of any one of embodiments 1-85, or a pharmaceutically acceptable salt thereof, or the pharmaceutical composition of any one of embodiments 86-87 in use for treating a liver disease in a subject in a subject.

127. The use of any one of embodiments 124-126, wherein the liver disease is selected from the group consisting of Hepatitis B, Hepatitis C, alcoholic liver disease, cirrhosis of the liver, hepatocellular carcinoma, and non-alcoholic steatohepatitis (NASH).

128. Use of a compound of any one of embodiments 1-85, or a pharmaceutically acceptable salt thereof, or the pharmaceutical composition of any one of embodiments 86-87 for treating a metabolic disease in a subject.

129. Use of a compound of any one of embodiments 1-85, or a pharmaceutically acceptable salt thereof, or the pharmaceutical composition of any one of embodiments 86-87 in the manufacture of a medicament for treating a metabolic disease in a subject.

130. A compound of any one of embodiments 1-85, or a pharmaceutically acceptable salt thereof, or the pharmaceutical composition of any one of embodiments 86-87 in use for treating a metabolic disease in a subject.

131. The use of any one of embodiments 128-130, wherein the metabolic disease is selected from the group consisting of metabolic syndrome, insulin resistance, Type II diabetes, porphyria, porphyria cutanea tarda, Wilson's Disease, and acute iron overdose.

132. Use of a compound of any one of embodiments 1-85, or a pharmaceutically acceptable salt thereof, or the pharmaceutical composition of any one of embodiments 86-87 for treating a neurodegenerative disorder in a subject.

133. Use of a compound of any one of embodiments 1-85, or a pharmaceutically acceptable salt thereof, or the pharmaceutical composition of any one of embodiments 86-87 in the manufacture of a medicament for treating a neurodegenerative disorder in a subject.

134. A compound of any one of embodiments 1-85, or a pharmaceutically acceptable salt thereof, or the pharmaceutical composition of any one of embodiments 86-87 in use for treating a neurodegenerative disorder in a subject.

135. Use of a compound of any one of embodiments 1-85, or a pharmaceutically acceptable salt thereof, or the pharmaceutical composition of any one of embodiments 86-87 for treating an infectious disease in a subject.

136. Use of a compound of any one of embodiments 1-85, or a pharmaceutically acceptable salt thereof, or the pharmaceutical composition of any one of embodiments 86-87 in the manufacture of a medicament for treating an infectious disease in a subject.

137. A compound of any one of embodiments 1-85, or a pharmaceutically acceptable salt thereof, or the pharmaceutical composition of any one of embodiments 86-87 in use for treating an infectious disease in a subject.

138. The use of any one of embodiments 135-137, wherein the infectious disease is a siderophilic infection.

139. The use of any one of embodiments 88-138, wherein the subject is a subject in need thereof.

140. Use of a compound of any one of embodiments 1-85, or a pharmaceutically acceptable salt thereof, or the pharmaceutical composition of any one of embodiments 86-87 for inhibiting matriptase 2, or a mutant thereof, in a biological sample.

141. A compound of any one of embodiments 1-85, or a pharmaceutically acceptable salt thereof, or the pharmaceutical composition of any one of embodiments 86-87 in use for inhibiting matriptase 2, or a mutant thereof, in a biological sample.

142. The use of any one of embodiments 88-141, wherein the compound of any one of embodiments 1-85, or a pharmaceutically acceptable salt thereof, or the pharmaceutical composition of any one of embodiments 86-87 is administered in an effective amount.

### EXAMPLES

### Synthesis

Compounds of the present disclosure, including salts thereof, can be prepared using known organic synthesis techniques and can be synthesized according to any of numerous possible synthetic routes.

The reactions for preparing compounds of the present disclosure can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures, which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H NMR or ¹³C NMR), infrared spectroscopy, spectrophotometry (e.g., UV-visible), or mass spectrometry, or by chromatography such as high performance liquid chromatography (“HPLC”) or thin layer chromatography, or liquid chromatography-mass spectrometry (“LC-MS”).

Unless otherwise stated, work-up includes distribution of the reaction mixture between the organic and aqueous phase indicated within parentheses, separation of layers and drying the organic layer over anhydrous sodium sulphate, filtration and distillation of the solvent under reduced pressure. Purification, unless otherwise mentioned, includes purification by silica gel chromatographic techniques, generally using ethyl acetate/petroleum ether mixture of a suitable polarity as the mobile phase.

Compounds of the present disclosure can be prepared using numerous preparatory reactions known in the literature. The Schemes below provide general guidance in connection with preparing the compounds provided herein. One skilled in the art would understand that the preparations shown in the Schemes can be modified or optimized using general knowledge of organic chemistry to prepare various compounds. Example synthetic methods for preparing compounds are provided in the schemes below.

### General Schemes:

The general synthetic methods used in each General Schemes follow and include an illustration of a compound that was synthesized using the designated General Scheme. None of the specific conditions and reagents noted herein are to be construed as limiting the scope of the present disclosure and are provided for illustrative purposes only.

The following abbreviations refer respectively to the definitions below:

The expressions, “ambient temperature,” “room temperature,” and “r.t.” as used herein, are understood in the art, and generally refer to a temperature, e.g., a reaction temperature, that is about the temperature of the room in which the reaction is carried out, for example, a temperature from about 20° C. to about 30° C.

ACN—Acetonitrile; br—Broad; ° C.—Degree Celsius; CHCl₃—Chloroform; CD₃OD—Deuterated Methanol; DMSO—d⁶—Deuterated dimethylsulfoxide; DCM—Dichloromethane; DIPEA—Diisopropylethylamine; DMF—N, N-Dimethylformamide; d—Doublet; dd—Doublet of doublet; EDC·HCl—1-(3-Dimethylaminopropyl)-3-ethylcarbodiimide hydrochloride; mg-Miligram; g—Gram; h—Hours; ¹H—Proton; HCl—Hydrochloric acid; HPLC—High-Performance Liquid Chromatography; H₂—Hydrogen; HOBt—1-Hydroxy benzotriazole; K₂CO₃—Potassium carbonate; LCMS—Liquid chromatography-mass spectroscopy; LiOH·H₂O—Lithium hydroxide monohydrate; M—Molar; MHz—Mega hertz (frequency); MeOH—Methanol; mL—MilliLiter; min—Minutes; mol—Moles; M⁺—Molecular ion; M—Multiplet; N₂—Nitrogen; NH₃—Ammonia; NBS—N-Bromosuccinimide; NCS—N-Chlorosuccinimide; NMR—Nuclear Magnetic Resonance; NaOH—Sodium Hydroxide; RT—Room temperature; s—Singlet; t—Triplet; TLC—Thin Layer Chromatography; TFA—Trifluoroacetic acid; TEA—Triethylamine; THF—Tetrahydrofuran; %—Percentage; μ—Micron; and δ—Delta; Zn—Zinc; mmol—millimoles.

Analysis for the compounds of the present disclosure, unless mentioned, was conducted in the general methods well known to the person skilled in the art. Having described the present disclosure with reference to certain preferred embodiments, other embodiments will become apparent to one skilled in the art from consideration of the specification. The present disclosure is further defined by reference to the following examples, describing in detail the analysis of the compounds of the present disclosure.

It will be apparent to those skilled in the art that many modifications, both to materials and methods, may be practiced without departing from the scope of the present disclosure.

### Materials and Methods

Unless otherwise stated, all reagents were obtained from commercial sources and were used as received without further purification. The NMR spectrometers utilized were Bruker instruments operating at frequencies between 400 and 600 MHz. UPLC-MS analysis was conducted on a Waters UPLC system with both Diode Array detection and Electrospray (+′ve and −′ve ion) MS detection. The mobile phase comprised H₂O containing 0.1% formic acid (A) and MeCN containing 0.1% formic acid (B) in the following linear gradients:

Method 1: 90% A (0.1 min), 90%-0% A (2.5 min), 0% A (0.3 min), 0-90% A (0.1 min) with a flow rate 0.5 mL/min. The stationary phase was a Waters Acquity UPLC BEH C18 1.7 um (2.1×50 mm) column

Method 2: 90% A (0.1 min), 90%-0% A (2.5 min), 0% A (0.3 min), 0-90% A (0.1 min) with a flow rate 0.5 mL/min. The stationary phase was a Waters Acquity UPLC BEH C4 1.7 um (2.1×100 mm) column, 300 A;

Method 3: 90% A (1 min), 90%-0% A (4 min), 0% A (0.5 min), 0-90% A (0.5 min) with a flow rate 0.5 mL/min. The stationary phase was a Waters Acquity UPLC BEH C4 1.7 um (2.1×100 mm) column, 300 A;

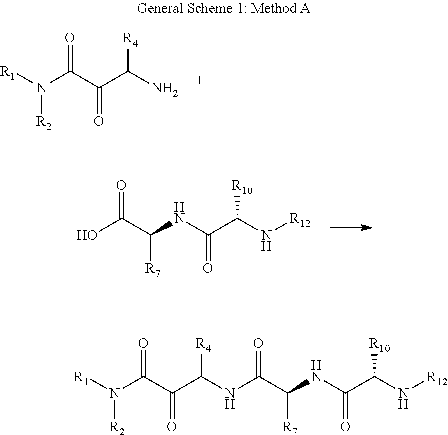

### Example 1: (S)—N-cyclopropyl-6-guanidino-3-((2S,3R)-3-hydroxy-2-((S)-4-phenyl-2-(3-phenylpropanamido)butanamido)butanamido)-2-oxohexanamide

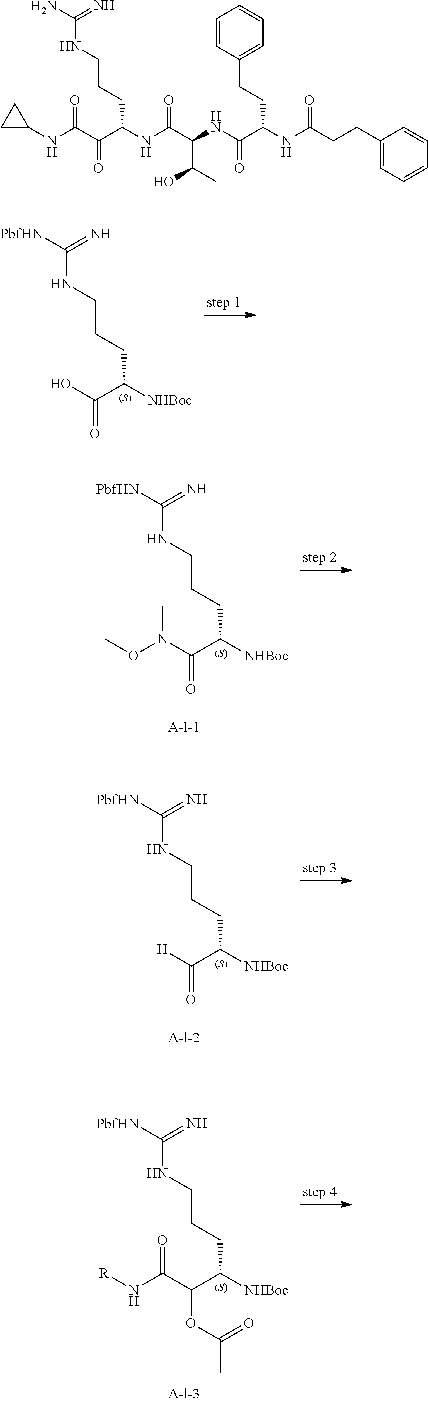

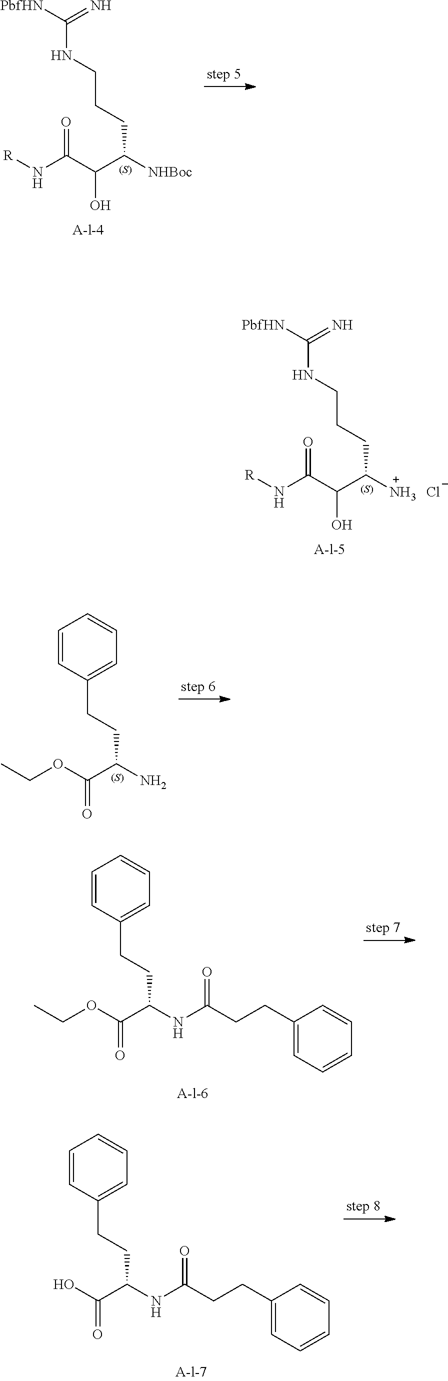

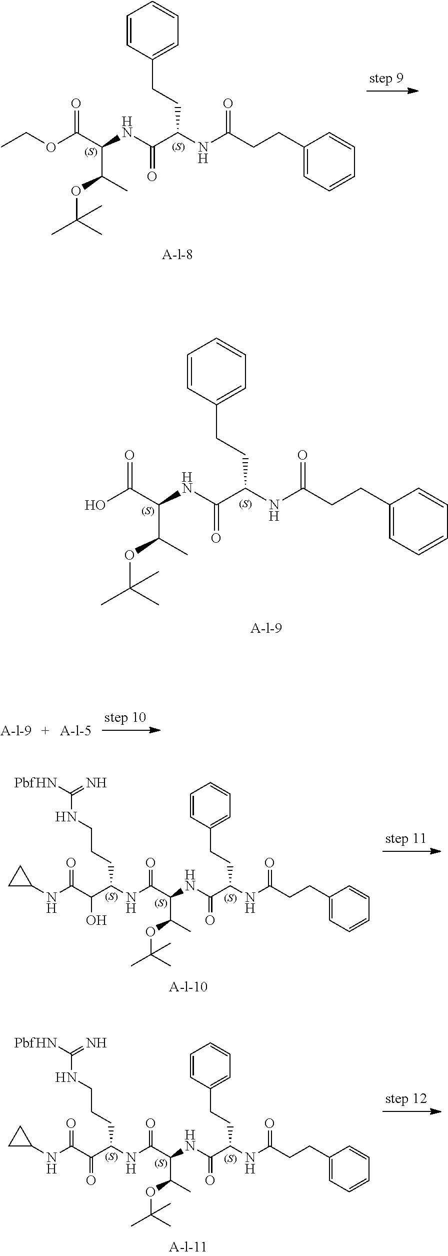

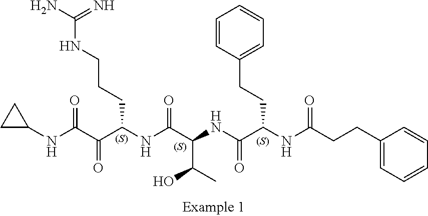

### Step-1: tert-butyl (S)-(1-(methoxy(methyl)amino)-1-oxo-5-(3-((2,2,4,6,7-pentamethyl-2,3-dihydrobenzofuran-5-yl)sulfonyl)guanidino)pentan-2-yl)carbamate A-I-1

To a stirred solution of Boc-Arg(Pbf)-OH (19.69 g, 37.39 mmol) in DMF (75 mL) were sequentially added HATU (17.06 g, 44.86 mmol), N-methoxymethanamine hydrochloride (4.38 g, 44.86 mmol) and DIPEA (23 mL, 130.86 mmol). After 2 h of stirring at room temperature, the reaction mixture was diluted with EtOAc and washed with HCl (1N), NaHCO₃ sat. solution, and brine. The organic phase was dried over Na₂SO₄, to afford after filtration and solvent removal compound A-I-1 as white powder (21 g, 100% yield) used as such in the following step. UPLC-MS (Method 1): Rt=1.86 min, m/z (ESI)⁺=570 [M+H]⁺.

### Step-2: tert-butyl (S)-(1-oxo-5-(3-((2,2,4,6,7-pentamethyl-2,3-dihydrobenzofuran-5-yl)sulfonyl)guanidino)pentan-2-yl)carbamate A-I-2

Compound A-I-1 (16.6 g, 29.14 mmol) was dissolved in dry THF (110 mL) under nitrogen and cooled to 0° C. LAH (20.1 ml, 1 M in THF) was added dropwise. At the end of the addition the cooling bath was removed and the reaction was stirred for an additional 30 min. During LAH addition, the reaction changes from solution to the gel-like mixture. The reaction was cooled back to 0° C. and EtOAc (380 ml) was added dropwise, followed by 760 ml of 1 M KHSO₄ water solution. The phases were separated, and organic layers were washed with 1M KHSO₄ (4×150 ml), Brine:NaHCO₃ sat. solution=brine 1:1 (1×150 ml) and sat. solution (1×150 ml). The organic layers were dried over Na₂SO₄ and evaporated to dryness to afford compound A-I-2 as a withe solid (14 g, 94% yield) used as such in the next step. UPLC-MS (Method 1): Rt=1.90 min, 1.99 min, m/z (ESI)⁺=511 [M+H]⁺

### Step-3: (3S)-3-((tert-butoxycarbonyl)amino)-1-(cyclopropylamino)-1-oxo-6-(3-((2,2,4,6,7-pentamethyl-2,3-dihydrobenzofuran-5-yl)sulfonyl)guanidino)hexan-2-yl acetate A-I-3

A stirred solution of A-I-2 (6.0 g, 11.75 mmol) in ethyl acetate (24 mL) at −10° C., was sequentially treated with cyclopropylisocyanide (0.9 mL, 14.1 mmol) and acetic acid (1.53 mL) keeping the temperature below 0° C. The reaction was stirred at room temperature for 16 h. The mixture was diluted with EtOAc (100 ml) and washed with 1N HCl (50 ml), NaHCO₃ sat. solution (50 ml), and brine (50 ml). The organic phase was dried over Na₂SO₄, filtered, and evaporated to afford a crude product, which was then purified by column chromatography using SiO₂ as an adsorbent and eluting with petroleum ether (“PE”)/EtOAc to afford compound A-I-3 as white solid (5.5 g, 73% yield) and a mixture of diastereoisomers. UPLC-MS (Method 1): Rt=1.85 min, 1.87 min; m/z (ESI)⁺=638 [M+H]⁺.

### Step-4: tert-butyl ((3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-yl)carbamate A-I-4

Compound A-I-3 (5.50 g, 8.62 mmol) was solved in MeOH/H₂O (1/1; 140 mL) and treated with K₂CO₃ (3.57 g, 25.87 mmol). The mixture was stirred for 1 h at rt, then diluted with EtOAc and the organic phase was washed with citric acid 10% w/w and brine. The resulting organic phase was then dried over Na₂SO₄, to afford after filtration and solvent removal compound A-I-4 as white solid in quantitative yield as a mixture of diastereoisomers. UPLC-MS (Method 1): Rt=1.76 min, 1.78 min; m/z (ESI)⁺=596 [M+H]⁺

### Step-5: (3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-aminium chloride A-I-5

Compound A-I-4 (4.9 g, 8.16 mmol) was dissolved in DCM (8 mL) and treated with a solution of HCl 2M in Et₂O (43.11 mL). The mixture was stirred at RT 1 h and then solvent was removed to afford compound A-I-5 in quantitative yield (4.9 g). UPLC-MS (Method 1): Rt=1.20 min; m/z (ESI)⁺=496 [M+H]⁺.

### Step-6: ethyl (S)-4-phenyl-2-(3-phenylpropanamido)butanoate A-I-6

To a solution of ethyl (2S)-2-amino-4-phenyl-butanoate hydrochloride (30.0 g, 123.09 mmol) in DMF (250 ml) were added 3-phenylpropanoic acid (22.18 g, 147.7 mmol), HATU (56.16 g, 147.7 mmol) and DIPEA (64 mL, 369.26 mmol). The mixture was stirred at room temperature for 16 h. The reaction mixture was diluted with EtOAc and the organic phase was washed sequentially with HCl (1N), NaHCO₃ sat. solution, and brine. The resulting organic phase was dried over Na₂SO₄, to afford, after filtration and solvent removal, a residue that was triturated with PE/EA=8/2 (300 ml) to afford after filtration compound A-I-6 (41.1 g, 98% yield) as off-white solid. UPLC-MS (Method 1): Rt=1.96 min, m/z (ESI)⁺=340 [M+H]⁺

### Step-7: (3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-aminium chloride A-I-7

Compound A-I-6 (20.0 g, 58.92 mmol) was dissolved in THF (180 mL) and Water (120 mL), and the resulting mixture was then cooled with ice bath and treated with lithium hydroxide monohydrate (2.97 g, 70.71 mmol). The mixture was stirred at room temperature. The solvents of the resulting mixture were partially evaporated and the pH of the mixture was adjusted to pH 3-4 with citric acid 10% w/w to form a suspension, which was isolated by filtration to afford compound A-I-7 (19.5 g, 92% yield) as a white solid. UPLC-MS (Method 1): Rt=1.63 min, m/z (ESI)⁺=312 [M+H]⁺.

### Step-8: methyl (2S,3R)-3-[(2-methylpropan-2-yl)oxy]-2-[[(2S)-4-phenyl-2-(3-phenylpropanoylamino)butanoyl]amino]butanoate A-I-8

To a stirred solution of compound A-I-7 (10.0 g, 32.12 mmol) in DMF (106 mL) were added O-tert-Butyl-L-threonine methyl ester hydrochloride (7.2 g, 32.12 mmol), HATU (11.12 g, 29.24 mmol), and DIPEA (16.78 mL, 96.35 mmol). The reaction mixture was stirred at room temperature for 2 h and then diluted with EtOAc and the organic phase washed sequentially with HCl (1N), NaHCO₃ sat. solution, and brine. The resulting organic phase was dried over Na₂SO₄, to afford after filtration and solvent removal compound A-I-8 (15.0 g, 98% yield) as an off-white solid. UPLC-MS (Method 1): Rt=2.09 min, m/z (ESI)+=483 [M+H]⁺.

### Step-9: O-(tert-butyl)-N—((S)-4-phenyl-2-(3-phenylpropanamido)butanoyl)-L-threonine A-I-9

Compound A-I-8 (14.0 g, 29.01 mmol) was dissolved in THF (53.23 mL) and Water (35.5 mL), the resulting mixture was cooled to 0° C. and treated with lithium hydroxide monohydrate (1.46 g, 34.81 mmol). The mixture was then stirred 30 min at 0° C. and then at room temperature for 1 h. The mixture was partially evaporated to form a residue, which was diluted with EtOAc and washed with citric acid 10% w/w. The resulting aqueous layer was extracted with EtOAc and the combined organic phases were washed with brine, dried over Na₂SO₄, filtered and evaporated to afford compound A-I-9 (12.9 g, 95% yield) as a white powder. UPLC-MS (Method 1): Rt=1.84 min, m/z (ESI)+=469 [M+H]⁺.

### Step-10: (3S)-3-((2S,3R)-3-(tert-butoxy)-2-((S)-4-phenyl-2-(3-phenylpropanamido)butanamido)butanamido)-N-cyclopropyl-2-hydroxy-6-(3-((2,2,4,6,7-pentamethyl-2,3-dihydrobenzofuran-5-yl)sulfonyl)guanidino)hexanamide A-I-10

Compound A-I-5 (4.6 g, 8.16 mmol) was dissolved in DMF (40.8 mL) to form a solution, to which were sequentially added compound A-I-9 (4.2 g, 8.97 mmol), HATU (3.4 g, 8.97 mmol) and DIPEA (4.97 mL, 28.55 mmol). The resulting mixture was stirred at room temperature for 1 h and then diluted with EtOAc and washed with HCl (1 N), NaHCO₃ sat. solution, and brine. The organic phase was dried over Na₂SO₄, filtered, and evaporated to afford compound A-I-10 (4.8 g, 62% yield) used as such in the following step. UPLC-MS (Method 1): Rt=2.21 min, m/z (ESI)+=947 [M+H]⁺

### Step-11: (S)-3-((2S,3R)-3-(tert-butoxy)-2-((S)-4-phenyl-2-(3-phenylpropanamido)butanamido)butanamido)-N-cyclopropyl-2-oxo-6-(3-((2,2,4,6,7-pentamethyl-2,3-dihydrobenzofuran-5-yl)sulfonyl)guanidino)hexanamide A-I-11

Compound A-I-10 (4.8 g, 5.09 mmol) was dissolved in Toluene (47 mL) and DMSO (47 mL) to form a solution, to which EDC HCl (4.54 g, 23.67 mmol) and 2,2-dichloroacetic acid (0.78 mL, 9.47 mmol) were sequentially added. The resulting mixture was stirred at room temperature for 1 h and diluted with EtOAc and washed with citric acid 10% w/w and brine. The organic phase was dried over Na₂SO₄, filtered, and evaporated to render a residue that was purified by column chromatography using C18 as an adsorbent and eluting with H₂O/MeCN+0.1% TFA to afford A-I-11 compound (2.18 g, 46% yield). UPLC-MS (Method 1): Rt=2.30 min, m/z (ESI)+=945 [M+H]⁺.

### Step-12: (S)—N-cyclopropyl-6-guanidino-3-((2S,3R)-3-hydroxy-2-((S)-4-phenyl-2-(3-phenylpropanamido)butanamido)butanamido)-2-oxohexanamide Example 1

Compound A-I-11 (778.0 mg, 0.82 mmol) was treated with 7.5 ml of a solution of TFA:H2O:TIPS=95:2.5:2.5 and stirred at RT 1 h. The resulting mixture was diluted with toluene and the solvents were removed under vacuum. The resulting crude product was purified by column chromatography using C18 as an adsorbent and eluting with H₂O/MeCN+0.1% TFA to afford compound Example 1 (300 mg, 56% yield) as white powder, TFA salt and mixture of diasteroisomers. UPLC-MS (Method 3): Rt=2.32 min, m/z (ESI)⁺=636 [M+H]⁺. ¹H NMR (DMSO-d6, 400 MHz+TFA) δ 8.7-8.5 (m, 1H), 8.2-8.1 (m, 2H), 7.9-8.1 (m, 1H), 8.01 (bd, 0.4H, J=8.7 Hz), 7.64 (br d, 0.6H, J=8.1 Hz), 7.64 (br d, 1H, J=8.1 Hz), 7.43 (br t, 1H, J=5.3 Hz), 7.3-6.6- (m, 14H), 4.9-5.1 (m, 1H), 4.4-4.1 (m, 2H), 4.0-3.7 (m, 1H), 3.09 (m, 2H), 2.9-2.8 (m, 2H), 2.8-2.7 (m, 1H), 2.5-2.4 (m, 4H overlap with DMSO), 2.1-1.7 (m, 3H), 1.6-1.3 (m, 3H), 1.05 (m, 3H), 0.7-0.4 (m, 4H).

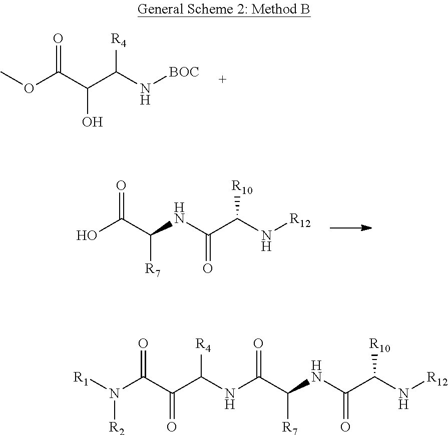

### Example 2: (S)-6-guanidino-3-((2S,3R)-3-hydroxy-2-((S)-4-phenyl-2-(3-phenylpropanamido)butanamido)butanamido)-N-(1-methylcyclopropyl)-2-oxohexanamide

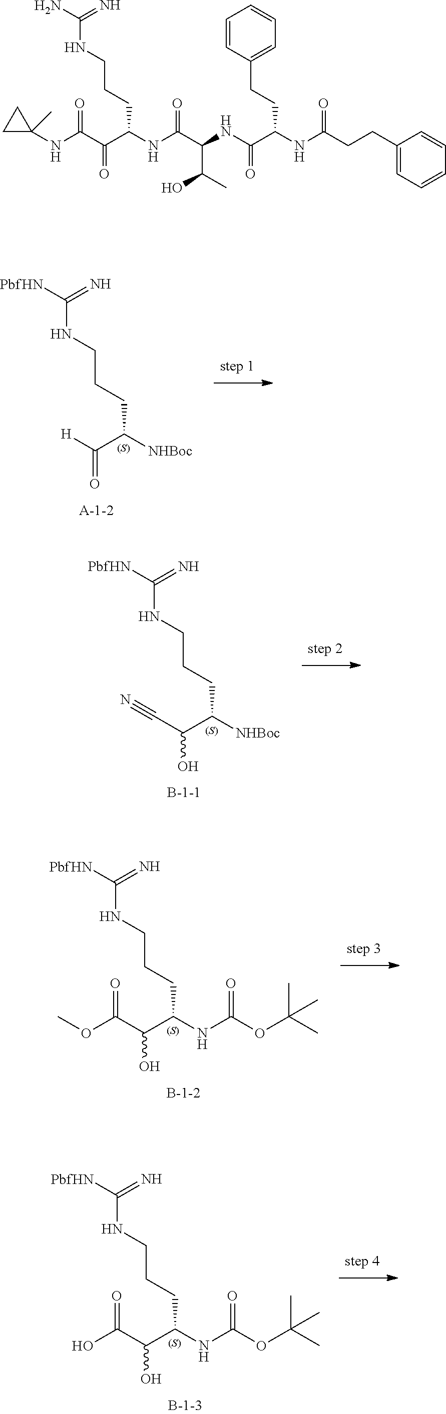

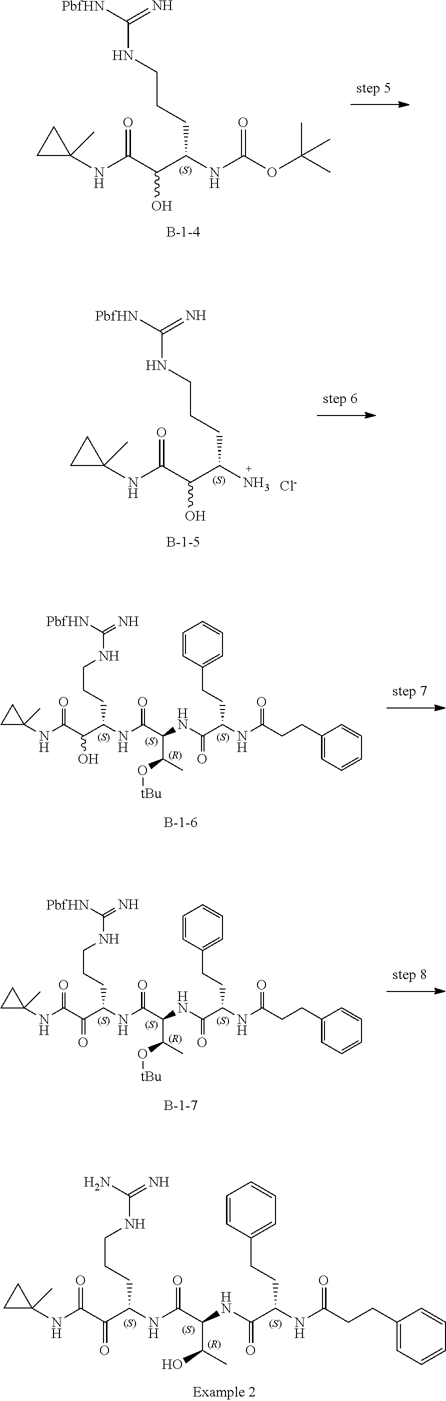

### Step-1: tert-butyl (1-cyano-1-hydroxy-5-(3-((2,2,4,6,7-pentamethyl-2,3-dihydrobenzofuran-5-yl)sulfonyl)guanidino)pentan-2-yl)carbamate B-I-1

A solution of compound A-I-2 (5.0 g, 9.79 mmol) in DCM (8.5 mL) was treated with acetone cyanohydrin (1.8 mL, 19.58 mmol). The mixture was stirred overnight at room temperature and then diluted with DCM and washed with HCl 1 N and brine. The organic phase was passed through a pad of SiO₂/Na₂SO₄/solka-flok washing with EtOAc. The resulting filtrate was dried over Na₂SO₄, filtered, and evaporated to compound B-I-1 (4.5 g, 59% yield) mixture of diastereoisomers used as such in the following step. UPLC-MS (Method 1): Rt=1.80 and 1.83 min, m/z (ESI)⁺=538 [M+H]⁺.

### Step-2: methyl (3S)-3-((tert-butoxycarbonyl)amino)-2-hydroxy-6-(3-((2,2,4,6,7-pentamethyl-2,3-dihydrobenzofuran-5-yl)sulfonyl)guanidino)hexanoate B-I-2

Compound B-I-1 (4.5 g, 5.86 mmol) was dissolved in MeOH (12 mL) and cooled to 0° C., to the resulting solution was added dropwise a solution of HCl 2M in Et₂O (30 mL). The reaction was left to warm up at room temperature and stirred overnight. The solvent was removed under vacuum to afford a pale orange residue that was dissolved in DCM (100 mL). To the resulting mixture were then added TEA (1.5 mL, 10.97 mmol) and tert-butoxycarbonyl tert-butyl carbonate (1.19 g, 5.49 mmol) sequentially. After overnight stirring, the mixture was diluted with DCM and washed with brine. The organic phase was dried over Na₂SO₄, to afford after filtration a residue that was purified by column chromatography using SiO₂ as an adsorbent and eluting with PE/EtOAc+10% MeOH to afford compound B-I-2 (1.0 g, 29% yield). UPLC-MS (Method 1): Rt=1.80 and 1.83 min, m/z (ESI)⁺=570 [M+H]⁺.

### Step-3: (3S)-3-((tert-butoxycarbonyl)amino)-2-hydroxy-6-(3-((2,2,4,6,7-pentamethyl-2,3-dihydrobenzofuran-5-yl)sulfonyl)guanidino)hexanoic acid B-I-3

A solution of lithium hydroxide monohydrate (13.34 mg, 0.560 mmol) in H₂O (2 mL) was added to a stirred solution of compound B-I-2 (265.0 mg, 0.460 mmol) in MeOH/THF: 2/1 (3 mL) and the reaction mixture was stirred overnight at room temperature. To drive the reaction for completion, an additional lithium hydroxide (13.34 mg, 0.560 mmol) in H₂O (0.5 mL) was added and the resulting mixture was stirred continued for 6 h. The reaction mixture was concentrated under reduced pressure to afford the crude product, which was purified by column chromatography using C18 as an adsorbent and eluting with H₂O/MeCN to afford compound B-I-3 (166 mg, 64% yield) as a mixture of diastereoisomers. UPLC-MS (Method 1): Rt=1.60 and 1.72 min, m/z (ESI)⁺=557 [M+H]⁺.

### Step-4: tert-butyl ((3S)-2-hydroxy-1-((1-methylcyclopropyl)amino)-1-oxo-6-(3-((2,2,4,6,7-pentamethyl-2,3-dihydrobenzofuran-5-yl)sulfonyl)guanidino)hexan-3-yl)carbamate B-I-4

To a stirred solution of compound B-I-3 (160.0 mg, 0.29 mmol) in DMF (3 mL) were added 1-methylcyclopropan-1-amine hydrochloride (61.84 mg, 0.57 mmol), HATU (196.72 mg, 0.52 mmol) and TEA (0.16 mL, 1.15 mmol). After 30 min of stirring at room temperature, the reaction mixture was diluted with EtOAc and washed with HCl (1N), NaHCO₃ sat. solution, and brine. The organic phase was dried over Na₂SO₄, to afford after filtration and solvent removal a residue that was purified by column chromatography using SiO₂ as an adsorbent and eluting with DCM/MeOH to afford compound B-I-4 (157 mg, 89% yield) as a mixture of diastereoisomers. UPLC-MS (Method 1): Rt=1.87 and 1.91 min, m/z (ESI)⁺=610 [M+H]⁺.

### Step-5: (3S)-2-hydroxy-1-((1-methylcyclopropyl)amino)-1-oxo-6-(3-((2,2,4,6,7-pentamethyl-2,3-dihydrobenzofuran-5-yl)sulfonyl)guanidino)hexan-3-aminium chloride B-I-5

To a stirred solution of B-I-4 (155.0 mg, 0.25 mmol) in DCM (1.3 mL) was added 2 M HCl solution in Et₂O (1.3 mL, 2.54 mmol). The mixture was stirred at RT for 1 h and then the volatiles was removed under vacuum to afford compound B-I-5 (143.0 mg, 100% yield) used as such in the following step. UPLC-MS (Method 1): Rt=1.23 min, m/z (ESI)+=510 [M+H]⁺.

### Step-6: (3S)-3-((2R,3R)-3-(tert-butoxy)-2-((S)-4-phenyl-2-(3-phenylpropanamido)butanamido)butanamido)-2-hydroxy-N-(1-methylcyclopropyl)-6-(3-((2,2,4,6,7-pentamethyl-2,3-dihydrobenzofuran-5-yl)sulfonyl)guanidino)hexanamide B-I-6

Compound A-I-9 (130.9 mg, 0.28 mmol) was dissolved in DMF (1.6 mL) and to the resulting solution was added compound B-I-5 (138.7 mg, 0.25 mmol), HATU (106.0 mg, 0.28 mmol) and DIPEA (0.27 mL, 1.52 mmol) sequentially. The resulting mixture was stirred at room temperature for 30 min and then diluted with EtOAc and washed with HCl (1N), NaHCO₃ sat. solution, and brine. The organic phase was dried over Na₂SO₄, to afford after filtration and solvent removal a residue that was purified by column chromatography using C18 as an adsorbent and eluting with H₂O/MeCN+0.1% TFA to afford compound B-I-6 (113 mg, 46% yield). UPLC-MS (Method 1): Rt=2.31 min, m/z (ESI)+=961 [M+H]⁺.

### Step-7: (S)-3-((2S,3R)-3-(tert-butoxy)-2-((S)-4-phenyl-2-(3-phenylpropanamido)butanamido)butanamido)-N-(1-methylcyclopropyl)-2-oxo-6-(3-((2,2,4,6,7-pentamethyl-2,3-dihydrobenzofuran-5-yl)sulfonyl)guanidino)hexanamide B-I-7

Dess Martin periodinane (59.9 mg, 0.14 mmol) was added to a stirred solution of compound B-I-6 (113.0 mg, 0.12 mmol) in DCM (5.9 mL, 0.092 mol. After 1 h of stirring at room temperature the reaction mixture was filtrated on a pad of solka-floc and concentrated under reduced pressure. The crude product was purified by column chromatography using SiO₂ as an adsorbent and eluting with DCM/MeOH to afford compound B-I-7 as a pale-yellow solid (93 mg, 36% yield). UPLC-MS (Method 1): Rt=2.40 min, m/z (ESI)+=959 [M+H]⁺.

### Step-8: (S)-6-guanidino-3-((2S,3R)-3-hydroxy-2-((S)-4-phenyl-2-(3-phenylpropanamido)butanamido)butanamido)-N-(1-methylcyclopropyl)-2-oxohexanamide Example 2

Compound B-I-7 (39.0 mg, 0.04 mmol) was treated with 0.5 ml of a solution of TFA:H₂O:TIPS=95:2.5:2.5. After 1 h of stirring at room temperature, the resulting mixture was diluted with toluene and solvent was removed under vacuum to afford the crude produce, which was purified by column chromatography using C18 as an adsorbent eluting with H₂O/MeCN+0.1% TFA to afford the titled compound Example 2 (20 mg, 55% yield) as white powder, TFA salt and mixture of diastereoisomers. UPLC-MS (Method 1): Rt=1.43 min, m/z (ESI)⁺=650 [M+H]⁺. ¹H-NMR (DMSO-d6, 400 MHz) δ 8.8-8.9 (m, 1H), 8.1-8.3 (m, 2H), 8.0-8.1 (m, 0.3H), 7.6-7.7 (m, 0.7H), 7.4-7.5 (m, 1H), 6.6-7.3 (m, 14H), 4.9-5.1 (m, 1H), 4.2-4.4 (m, 2H), 3.8-4.0 (m, 1H), 3.0-3.2 (m, 2H), 2.8-3.0 (m, 2H), 2.4-2.5 (m, 4H), 1.7-2.0 (m, 3H), 1.4-1.6 (m, 3H), 1.2-1.3 (m, 3H), 1.0-1.1 (m, 3H), 0.6-0.7 (m, 2H), 0.5-0.6 (m, 2H).

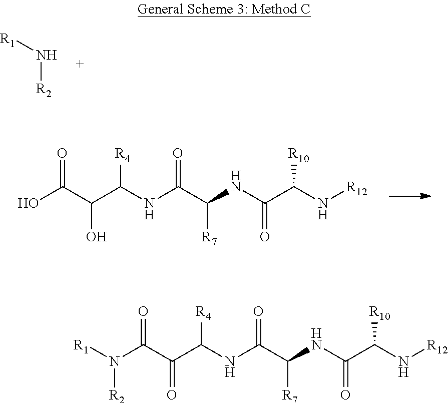

### Example 3 6-guanidino-3-((2S,3R)-3-hydroxy-2-((S)-4-phenyl-2-(3-phenylpropanamido)butanamido)butanamido)-2-oxo-N-(pyridin-3-ylmethyl)hexanamide

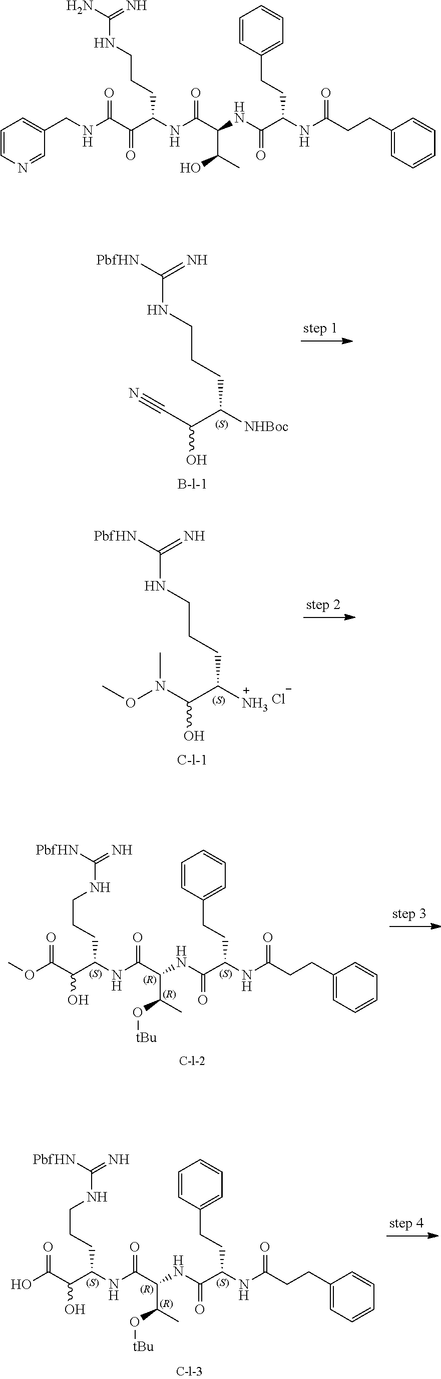

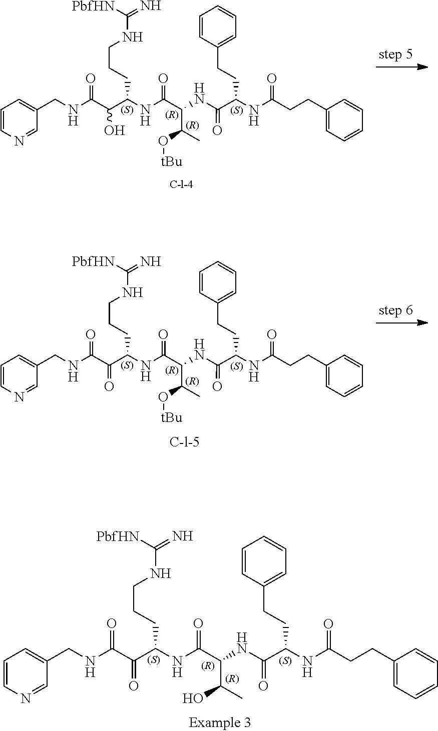

### Step-1: (3S)-2-hydroxy-1-methoxy-1-oxo-6-(3-((2,2,4,6,7-pentamethyl-2,3-dihydrobenzofuran-5-yl)sulfonyl)guanidino)hexan-3-aminium chloride C-I-1

A solution of HCl 2 M in Et₂O (29.48 mL, 58.96 mmol) was added dropwise to a stirred solution of compound B-I-1 (3.17 g, 5.9 mmol) in MeOH (12.0 mL) at 0° C. The reaction mixture was left to warm up at RT. After overnight stirring, the reaction mixture was treated with 30 ml of ice and stirred for 30 min and then cooled to 0° C. and, to the mixture, Na₂CO₃ was added portion wise to reach pH 9-10. The basic aqueous phase was extracted with EtOAc. The combined organic layers were washed with brine, dried over Na₂SO₄ to afford after filtration and evaporation compound C-I-1 as pale orange powder (1.9 g, 68% yield). UPLC-MS (Method 1): Rt=1.29 min, m/z (ESI)⁺=471 [M+H]⁺

### Step-2: methyl (3S)-3-((2R,3R)-3-(tert-butoxy)-2-((S)-4-phenyl-2-(3-phenylpropanamido)butanamido)butanamido)-2-hydroxy-6-(3-((2,2,4,6,7-pentamethyl-2,3-dihydrobenzofuran-5-yl)sulfonyl)guanidino)hexanoate C-I-2

A mixture of compound C-I-1 (1.9 g, 4.04 mmol) and compound A-I-9 (2.0, 4.24 mmol) in DMF (10 mL) was treated with HATU (1.6 g, 4.24 mmol) and DIPEA (2.11 mL, 12.11 mmol). After overnight stirring, the reaction mixture was diluted with EtOAc and washed with HCl (1N), NaHCO₃ sat. solution, and brine. The organic layer was dried over Na₂SO₄, to afford, after filtration and solvent removal, compound C-I-2 used as such in the next step (3.3 g, 88% yield). UPLC-MS (Method 1): Rt=2.22 min, m/z (ESI)⁺=922 [M+H]⁺

### Step-3: (3S)-3-((2R,3R)-3-(tert-butoxy)-2-((S)-4-phenyl-2-(3-phenylpropanamido)butanamido)butanamido)-2-hydroxy-6-(3-((2,2,4,6,7-pentamethyl-2,3-dihydrobenzofuran-5-yl)sulfonyl)guanidino)hexanoic acid C-I-3

Compound C-I-2 (1170.0 mg, 1.27 mmol) was dissolved in THF (10 mL) and Water (5 mL). To the resulting mixture, lithium hydroxide monohydrate (63.9 mg, 1.52 mmol) was added, and the mixture was stirred for 1 h. The mixture was cooled to 0° C. and acidified with citric acid 10% w/w and then extracted with EtOAc. The combined organic phases were washed with brine and dried over Na₂SO₄, to afford after filtration compound C-I-3 used as such (1000 mg, 86% yield). UPLC-MS (Method 1): Rt=2.11 min, m/z (ESI)⁺=908 [M+H]⁺

### Step-4: (3S)-3-((2R,3R)-3-(tert-butoxy)-2-((S)-4-phenyl-2-(3-phenylpropanamido)butanamido)butanamido)-2-hydroxy-6-(3-((2,2,4,6,7-pentamethyl-2,3-dihydrobenzofuran-5-yl)sulfonyl)guanidino)-N-(pyridin-3-ylmethyl)hexanamide C-I-4

To a stirred solution of compound C-I-3 (300.0 mg, 0.330 mmol) in DMF (1.5 mL) were added 3-pyridylmethylamine (0.07 mL, 0.66 mmol) HATU (226.34 mg, 0.60 mmol) and TEA (0.18 mL, 1.32 mmol). After overnight stirring, the reaction mixture was diluted with EtOAc and washed with citric acid 10% w/w, NaHCO₃ sat. solution, and brine. The organic phase was dried over Na₂SO₄, to afford after filtration and solvent removal the crude product. The crude product was purified by column chromatography using SiO₂-diol as an adsorbent and eluting with PE/Isopropanol to afford compound C-I-4 (200 mg, 30% yield). UPLC-MS (Method 1): Rt=1.88 and 1.91 min, m/z (ESI)⁺=998 [M+H]⁺

### Step-5: (S)-3-((2R,3R)-3-(tert-butoxy)-2-((S)-4-phenyl-2-(3-phenylpropanamido)butanamido)butanamido)-2-oxo-6-(3-((2,2,4,6,7-pentamethyl-2,3-dihydrobenzofuran-5-yl)sulfonyl)guanidino)-N-(pyridin-3-ylmethyl)hexanamide C-I-5

Compound C-I-4 (100.0 mg, 0.10 mmol) was dissolved in DMSO (1.0 mL) and Toluene (1.0 mL) and to the resulting mixture were added EDC HCl (192.23 mg, 1.0 mmol) and 2,2-dichloroacetic acid (0.03 mL, 0.40 mmol). After 30 min of stirring at room temperature, the reaction was diluted with EtOAc and washed with water and brine. The organic phase was dried over Na₂SO₄, to afford after filtration and solvent removal a residue that was purified by column chromatography using C18 as an adsorbent and eluting with H₂O/MeCN+0.1% TFA to afford compound C-I-5 (80.0 mg, yield 80%). UPLC-MS (Method 1): Rt=1.97 min, m/z (ESI)⁺=996 [M+H]⁺

### Step-6: ((S)-6-guanidino-3-((2R,3R)-3-hydroxy-2-((S)-4-phenyl-2-(3-phenylpropanamido)butanamido)butanamido)-2-oxo-N-(pyridin-3-ylmethyl)hexanamide Example 3

Compound C-I-5 (80.0 mg, 0.080 mmol) was treated with 0.5 ml of a solution of TFA:H₂O:TIPS=95:2.5:2.5. After 1 h of stirring at room temperature, the resulting mixture was diluted with toluene and volatiles were removed under vacuum. The resulting crude was purified by column chromatography using C18 as an adsorbent eluting with H₂O/MeCN+0.1% TFA to afford the titled compound Example 3 (19 mg, 34% yield) as white powder, TFA (2×) salt and mixture of diastereoisomers. UPLC-MS (Method 3): Rt=2.04 min, m/z (ESI)⁺=687 [M+H]⁺. 1H NMR (DMSO-d6, 400 MHz) δ 9.3-9.5 (m, 1H), 8.7-8.9 (m, 2H), 8.4-8.5 (m, 1H), 8.1-8.3 (m, 2H), 8.0-8.1 (m, 2H), 7.6-7.7 (m, 1H), 7.4-7.5 (m, 1H), 6.7-7.4 (m, 14H), 4.9-5.1 (m, 1H), 4.4-4.6 (m, 2H), 4.1-4.4 (m, 2H), 3.8-4.1 (m, 1H), 3.0-3.2 (m, 2H), 2.7-2.9 (m, 2H), 2.5 (m, 4H overlap DMSO), 1.7-2.0 (m, 3H), 1.4-1.7 (m, 3H), 0.9-1.1 (m, 3H).

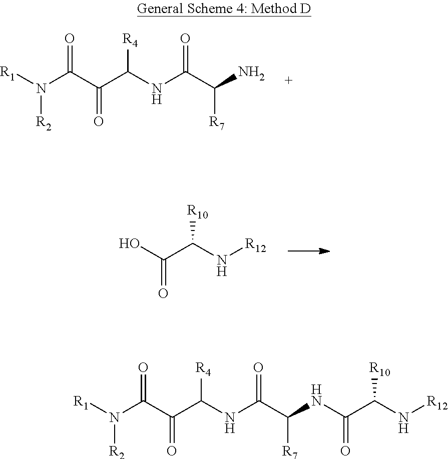

### Example 4: (2S,4R)-4-(benzyloxy)-N—((S)-1-(cyclopropylamino)-6-guanidino-1,2-dioxohexan-3-yl)-1-((S)-4-phenyl-2-(3-phenylpropanamido)butanoyl)pyrrolidine-2-carboxamide

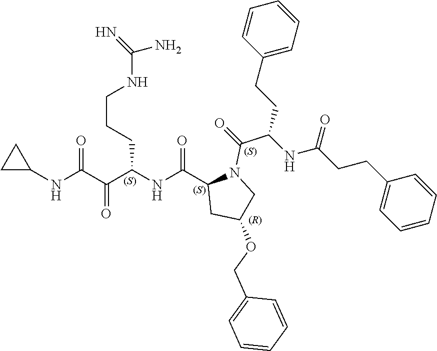

### Example 5: (2S,4R)—N—((S)-1-(cyclopropylamino)-6-guanidino-1,2-dioxohexan-3-yl)-4-hydroxy-1-((S)-4-phenyl-2-(3-phenylpropanamido)butanoyl)pyrrolidine-2-carboxamide

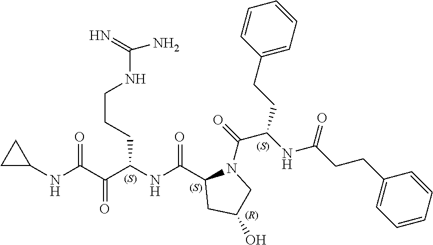

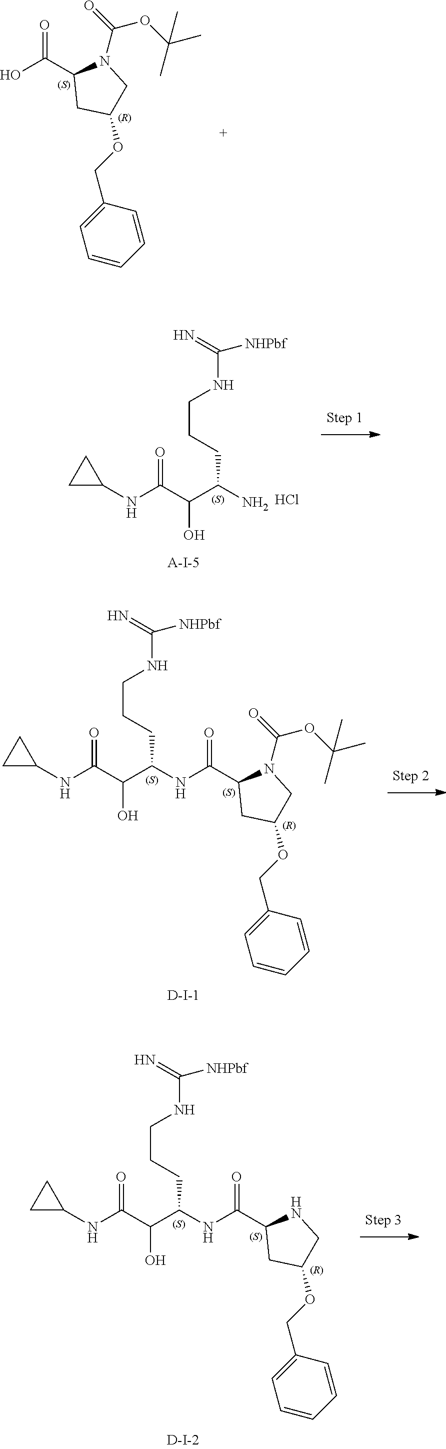

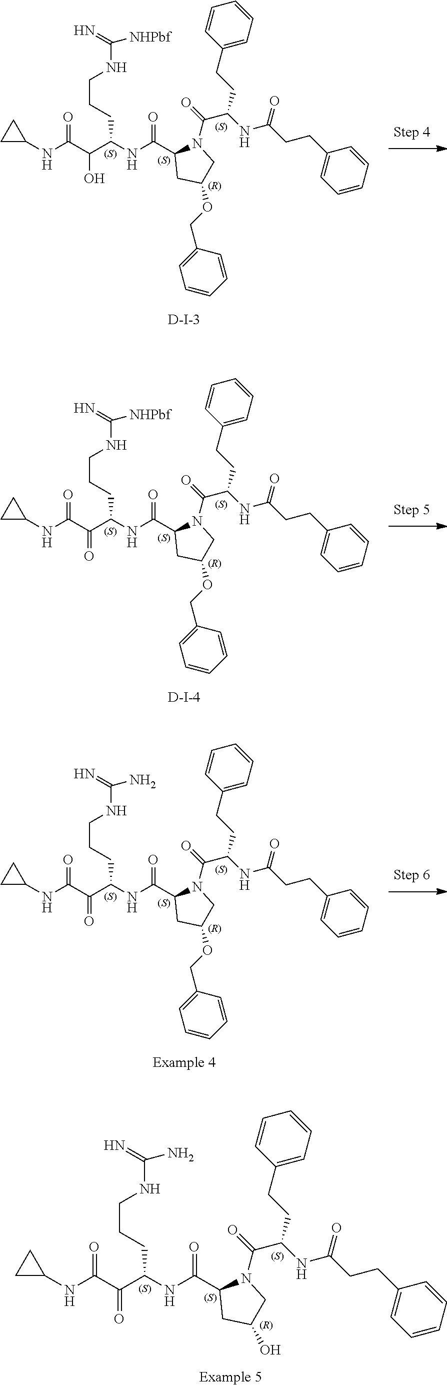

### Step-1: tert-butyl (2S,4R)-4-(benzyloxy)-2-(((3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-yl)carbamoyl)pyrrolidine-1-carboxylate D-I-1

To a stirred solution of (2S,4R)-1-[(2-methylpropan-2-yl)oxycarbonyl]-4-phenylmethoxy-pyrrolidine-2-carboxylic acid (310.88 mg, 0.970 mmol) in DMF (5.5 mL) were added HATU (401.26 mg, 1.06 mmol), DIPEA (0.77 mL, 4.4 mmol) and compound A-I-5 (500.0 mg, 0.88 mmol). After 30 min of stirring at room temperature, the mixture was diluted with EtOAc and washed with HCl (1N), NaHCO₃ sat. solution, and brine. The organic phase was dried over Na₂SO₄, to afford after filtration and solvent removal a residue that was purified by column chromatography using SiO₂ as an adsorbent and eluting with DCM/MeOH to afford compound D-I-1 as white solid (240.0 mg; 34% yield). UPLC-MS (Method 1): Rt=2.03 min, m/z (ESI)⁺=800 [M+H]⁺

### Step-2: (2S,4R)-4-(benzyloxy)-2-(((3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-yl)carbamoyl)pyrrolidin-1-ium chloride D-I-2

Compound D-I-1 (670.0 mg, 0.84 mmol) was dissolved in DCM (4.2 mL) and treated with HCl 2M in Et2O (4.2 mL, 8.39 mmol). The resulting mixture was stirred for 1 h at room temperature. The solvents were removed under reduced pressure to afford compound D-I-2 as dark yellow sticky solid (690.0 mg, 100% yield). UPLC-MS (Method 1): Rt=1.36 min, m/z (ESI)⁺=700 [M+H]⁺

### Step-3: (2S,4R)-4-(benzyloxy)-N-((3S)-1-(cyclopropylamino)-2-hydroxy-1-oxo-6-(3-((2,2,4,6,7-pentamethyl-2,3-dihydrobenzofuran-5-yl)sulfonyl)guanidino)hexan-3-yl)-1-((S)-4-phenyl-2-(3-phenylpropanamido)butanoyl)pyrrolidine-2-carboxamide D-I-3

A solution of (2S)-4-phenyl-2-(3-phenylpropanoylamino)butanoic acid (287.71 mg, 0.920 mmol) in DMF (5.25 mL) was treated with HATU (351.33 mg, 0.920 mmol), DIPEA (325.69 mg, 2.52 mmol) and compound D-I-2 (617.68 mg, 0.840 mmol). After 30 min of stirring at room temperature, the mixture was diluted with EtOAc and washed with HCl (1N), NaHCO₃ sat. solution, and brine. The organic phase was dried over Na₂SO₄, to afford after filtration and solvent removal a residue that was purified by column chromatography using SiO₂ as an adsorbent and eluting with DCM/MeOH to afford compound D-I-3 as pale-yellow solid (570.0 mg; 68% yield). UPLC-MS (Method 1): Rt=2.23 min, m/z (ESI)⁺=993 [M+H]⁺

### Step-4: (2S,4R)-4-(benzyloxy)-N—((S)-1-(cyclopropylamino)-1,2-dioxo-6-(3-((2,2,4,6,7-pentamethyl-2,3-dihydrobenzofuran-5-yl)sulfonyl)guanidino)hexan-3-yl)-1-((S)-4-phenyl-2-(3-phenylpropanamido)butanoyl)pyrrolidine-2-carboxamide D-I-4

Compound D-I-3 (570.0 mg, 0.570 mmol) was dissolved in Toluene (5.7 mL) and DMSO (5.7 mL), then EDC HCl (1101.25 mg, 5.74 mmol) and 2,2-dichloroacetic acid (0.19 mL, 2.3 mmol) were added. After 30 min of stirring at room temperature, the mixture was diluted with EtOAc and washed with citric acid 10% w/w and brine. The organic phase was dried over Na₂SO₄, to afford after filtration and solvent removal a residue that was purified by column chromatography using C18 as an adsorbent and eluting with H₂O/MeCN+0.1% TFA to afford compound D-I-4 as white solid (490.0 mg; 86% yield) and a mixture of diastereoisomers. UPLC-MS (Method 1): Rt=2.23 and 2.32 min, m/z (ESI)⁺=991 [M+H]⁺

### Step-5: ((2S,4R)-4-(benzyloxy)-N—((S)-1-(cyclopropylamino)-6-guanidino-1,2-dioxohexan-3-yl)-1-((S)-4-phenyl-2-(3-phenylpropanamido)butanoyl)pyrrolidine-2-carboxamide Example 4

Compound D-I-4 (490.0 mg, 0.49 mmol) was treated with 5.0 ml of a solution of TFA:H2O:TIPS=95:2.5:2.5. After 1 h of stirring at room temperature, the resulting mixture was diluted with toluene and solvent was removed under vacuum. The resulting crude was purified by column chromatography using C18 as an adsorbent eluting with H₂O/MeCN+0.1% TFA to afford the titled compound Example 4 (266 mg, 55% yield) as white powder, TFA salt and mixture of diastereoisomers. UPLC-MS (Method 1): Rt=1.76 min, m/z (ESI)⁺=738 [M+H]⁺. 1H NMR (DMSO-d6, 400 MHz) δ 8.6-8.8 (m, 1H), 8.4-8.5 (m, 1H), 8.0-8.2 (m, 1H), 7.4-7.5 (m, 1H), 7.1-7.4 (m, 19H), 4.8-5.1 (m, 1H), 4.4-4.5 (m, 4H), 4.1-4.4 (m, 1H), 3.7-3.9 (m, 1H), 3.4-3.5 (m, 1H), 3.0-3.2 (m, 2H), 2.7-2.9 (m, 3H), 2.3-2.5 (m, 4H), 2.2-2.3 (m, 1H), 1.7-1.9 (m, 4H), 1.4-1.7 (m, 3H), 0.5-0.7 (m, 4H)

### Step-6: (2S,4R)—N—((S)-1-(cyclopropylamino)-6-guanidino-1,2-dioxohexan-3-yl)-4-hydroxy-1-((S)-4-phenyl-2-(3-phenylpropanamido)butanoyl)pyrrolidine-2-carboxamide

### Example 5

To stirred solution of compound Example 4 (50.0 mg, 0.070 mmol) in MeOH (1 mL) and HCl (0.01 mL, 1N) was added Pd/C. The resulting mixture was stirred overnight under hydrogen atmosphere at room temperature. The reaction mixture was filtered through a pad of Solka-Floc, then solvent was removed under reduced pressure. The resulting crude product was purified by column chromatography using C18 as an adsorbent eluting with H₂O/MeCN+0.1% TFA to afford the titled compound Example 5 (12.0 mg, 20% yield) as white powder, TFA salt and mixture of diastereoisomers. UPLC-MS (Method 1): Rt=1.35 min, m/z (ESI)⁺=648 [M+H]⁺. 1H NMR (DMSO-d₆+TFA, 400 MHz) δ 8.6-8.8 (m, 1H), 8.3-8.5 (m, 1H), 8.0-8.1 (m, 1H), 7.6-7.7 (m, 1H), 7.4-7.5 (m, 1H), 7.1-7.3 (m, 14H), 4.8-5.0 (m, 1H), 4.2-4.5 (m, 3H), 3.3-3.5 (m, 2H), 3.0-3.2 (m, 2H), 2.6-2.9 (m, 3H), 2.3-2.5 (m, 4H), 2.6-2.6 (m, 1H), 1.7-1.9 (m, 4H), 1.7-1.9 (m, 3H), 0.5-0.7 (m, 4H).

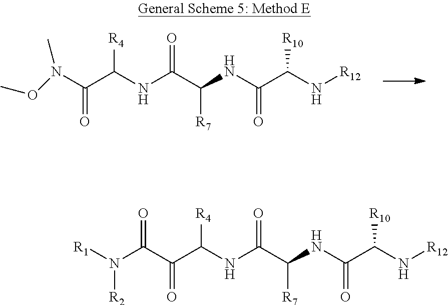

### Example 6: (3S)-4-(1-carbamimidoylpiperidin-4-yl)-N-cyclopropyl-2-oxidanylidene-3-[[(2S,3R)-3-oxidanyl-2-[[(2S)-4-phenyl-2-(3-phenylpropanoylamino)butanoyl]amino]butanoyl]amino]butanamide

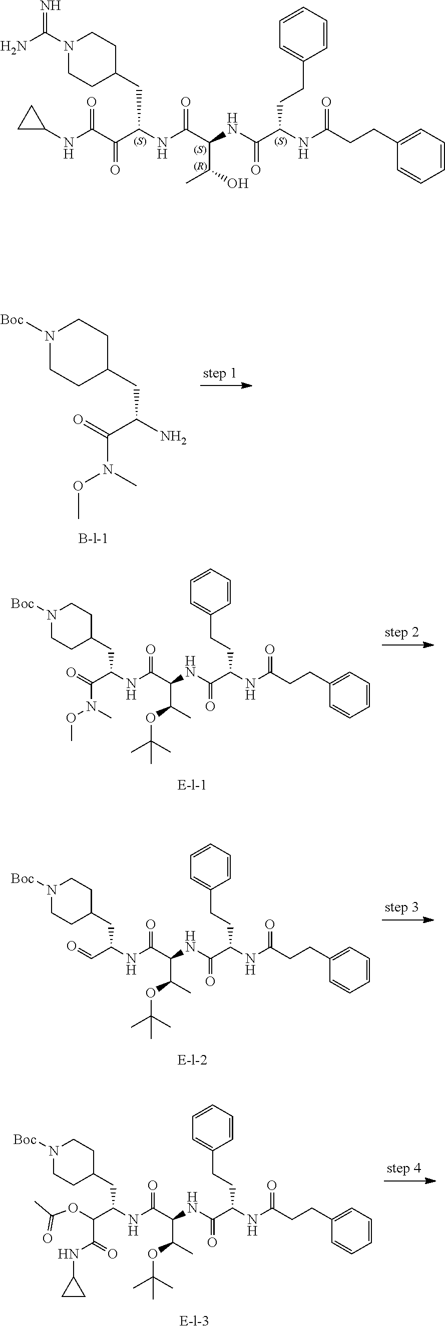

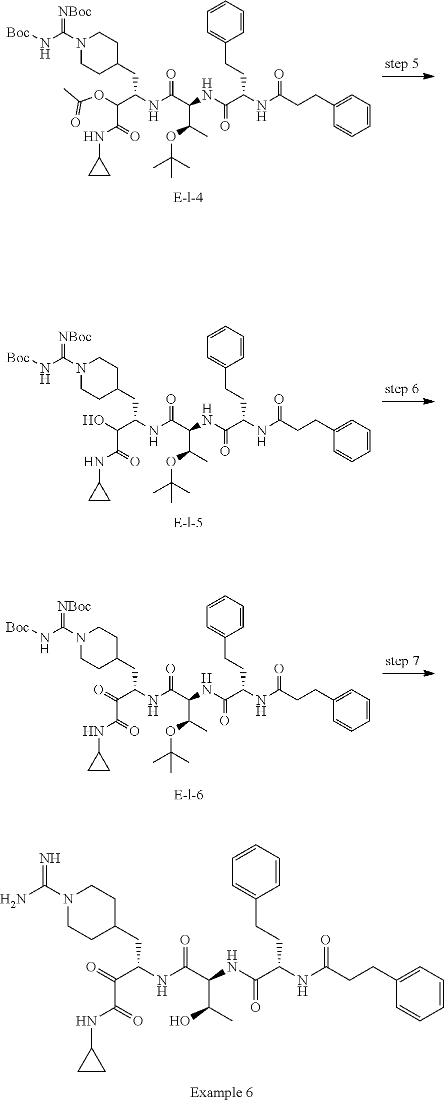

### Step-1: tert-butyl 4-((S)-2-((2S,3R)-3-(tert-butoxy)-2-((S)-4-phenyl-2-(3-phenylpropanamido)butanamido)butanamido)-3-(methoxy(methyl)amino)-3-oxopropyl)piperidine-1-carboxylate E-I-1

To a stirred solution of compound A-I-9 (900.0 mg, 1.92 mmol) in DMF (16 mL) were added HATU (900.0 mg, 2.37 mmol), tert-butyl 4-[(2R)-2-azanyl-3-[methoxy(methyl)amino]-3-oxidanylidene-propyl]piperidine-1-carboxylate (605.79 mg, 1.92 mmol) and DIPEA (1.2 mL, 6.96 mmol). After 2 h of stirring at room temperature, the reaction was diluted with EtOAc and washed with HCl (1N), NaHCO3 sat. solution, and brine. The organic phase was dried over Na₂SO₄, to afford after filtration and solvent removal the titled compound used as such in the following step (1500.0 mg, 100% yield). UPLC-MS (Method 1): Rt=2.34 min, m/z (ESI)⁺=789 [M+Na]⁺

### Step-2: tert-butyl 4-((S)-2-((2S,3R)-3-(tert-butoxy)-2-((S)-4-phenyl-2-(3-phenylpropanamido)butanamido)butanamido)-3-oxopropyl)piperidine-1-carboxylate E-I-2

Compound E-I-1 (900.0 mg, 1.17 mmol) was dissolved in dry THF (19 mL) under nitrogen and cooled to 0° C. LAH (0.82 ml, 1 M in THF) was added dropwise. At the end of the addition, the cooling bath was removed and the reaction mixture was stirred for additional 30 min. During LAH addition the reaction changes from solution to gel like mixture. The reaction was cooled back to 0° C. and EtOAc was added dropwise, followed by KHSO₄ (1 M). The phases were separated, and organic layers were washed with KHSO₄ (1M), Brine/NaHCO₃ sat. solution, =1/1 and brine. The organic layers were dried over Na₂SO₄ and evaporated to dryness to afford the titled compound as a withe solid (670.0 mg, 81% yield) used as such in the next step. UPLC-MS (Method 1): Rt=1.64 min, m/z (ESI)⁺ no ionization.

### Step-3: tert-butyl 4-((2S)-3-acetoxy-2-((2S,3R)-3-(tert-butoxy)-2-((S)-4-phenyl-2-(3-phenylpropanamido)butanamido)butanamido)-4-(cyclopropylamino)-4-oxobutyl)piperidine-1-carboxylate E-I-3

A stirred solution of E-I-2 (670.mg, 0.950 mmol) in EtOAc (2.4 mL) at −10° C., was sequentially treated with isocyanocyclopropane (0.073 mL, 1.13 mmol) and acetic acid (0.12 mL) keeping the temperature below 0° C. The reaction mixture was stirred at room temperature for 12 h. The mixture was diluted with EtOAc and washed with HCl (1N), NaHCO₃ sat. solution, and brine. The organic phase dried over Na₂SO₄, to afford after filtration and solvent removal the crude product that was purified by column chromatography using SiO₂ as an adsorbent and eluted with PE/EtOAc to afford compound E-I-3 as white solid (240.0 mg, 30% yield) and mixture of diastereoisomers. UPLC-MS (Method 1): Rt=2.28 and 2.32 min; m/z (ESI)−=833 [M−H]⁺.

### Step-4: (3S)-4-(1-((E)-N,N′-bis(tert-butoxycarbonyl)carbamimidoyl)piperidin-4-yl)-3-((2S,3R)-3-(tert-butoxy)-2-((S)-4-phenyl-2-(3-phenylpropanamido)butanamido)butanamido)-1-(cyclopropylamino)-1-oxobutan-2-yl acetate E-I-4

To a stirred solution of compound E-I-3 (230.mg, 0.280 mmol) in 2 ml of DCM/TFA=95/5 and stirred at room temperature for 3 h. The solvents were co-evaporated with toluene under vacuum to afford a residue that was dissolved in THF (3.0 mL) and treated with TEA (0.11 mL, 0.820 mmol) followed by N,N′-Di-Boc-1H-pyrazole-1-carboxamidine (126.86 mg, 0.41 mmol). After overnight stirring at room temperature, solvents were evaporated and the resulting residue was purified by column chromatography using SiO₂ as an adsorbent and eluted with PE/EtOAc to afford compound E-I-4 as white foam (130.0 mg, 45% yield). UPLC-MS (Method 1): Rt=1.86 and 1.98 min; m/z (ESI)⁺=977 [M+H]⁺.

### Step-5: tert-butyl ((E)-(4-((2S)-2-((2S,3R)-3-(tert-butoxy)-2-((S)-4-phenyl-2-(3-phenylpropanamido)butanamido)butanamido)-4-(cyclopropylamino)-3-hydroxy-4-oxobutyl)piperidin-1-yl)((tert-butoxycarbonyl)amino)methylene)carbamate E-I-5

A solution of K₂CO₃ (55.13 mg, 0.40 mmol) in H₂O (1.1 mL) was added to a stirred solution of compound E-I-4 (130.0 mg, 0.13 mmol) in MeOH (1.1 mL). After 20 min of stirring at room temperature, the mixture was diluted with EtOAc and washed with citric acid 10% w/w and brine. The organic phase was dried over Na₂SO₄, to afford after filtration and solvent removal compound E-I-5 as yellow solids (125.0 mg, 100% yield) used as such in the following step. UPLC-MS (Method 1): Rt=1.91 and 1.93 min; m/z (ESI)⁺=935 [M+H]⁺.

### Step-6: tert-butyl ((E)-(4-((S)-2-((2S,3R)-3-(tert-butoxy)-2-((S)-4-phenyl-2-(3-phenylpropanamido)butanamido)butanamido)-4-(cyclopropylamino)-3,4-dioxobutyl)piperidin-1-yl)((tert-butoxycarbonyl)amino)methylene)carbamate E-I-6

To a stirred solution of E-I-5 (58.0 mg, 0.06 mmol) in Toluene (0.6 mL) and DMSO (0.6 mL) were added EDC HCl (119.02 mg, 0.620 mmol) and 2,2-dichloroacetic acid (0.02 mL, 0.250 mmol). The reaction mixture was stirred 30 min at room temperature and then diluted with EtOAc, washed with citric acid 10% w/w and brine. The organic phase was dried over Na₂SO₄, filtered, and evaporated to afford compound E-I-6 used as such in the following step (57.0 mg, 97% yield). UPLC-MS (Method 1): Rt=2.03 min; m/z (ESI)⁺=933 [M+H]⁺.

### Step-7: (3S)-4-(1-carbamimidoylpiperidin-4-yl)-N-cyclopropyl-2-oxidanylidene-3-[[(2S,3R)-3-oxidanyl-2-[[(2S)-4-phenyl-2-(3-phenylpropanoylamino)butanoyl]amino]butanoyl]amino]butanamide Example 6

A stirred solution of compound E-I-6 (57.0 mg, 0.06 mmol) in DCM (0.6 mL) was treated with TFA (0.4 mL) and stirred at room temperature for 2 h. After solvent removal, residue was purified by column chromatography using C18 as an adsorbent and eluted with H₂O/MeCN+0.1% TFA to afford the titled compound Example 6 as white powder (11.0 mg, 26% yield). UPLC-MS (Method 3): Rt=2.35 min; m/z (ESI)⁺=676 [M+H]⁺. ¹H NMR (DMSO-d6+TFA, 400 MHz) δ 8.72 (br d, 1H, J=5.0 Hz), 8.19 (br d, 1H, J=7.7 Hz), 8.1-8.1 (m, 1H), 7.67 (br d, 1H, J=8.1 Hz), 7.1-7.3 (m, 14H), 5.1-5.2 (m, 1H), 4.2-4.4 (m, 1H), 4.17 (dd, 1H, J=4.6, 7.9 Hz), 3.9-4.0 (m, 1H), 3.78 (br d, 2H, J=12.9 Hz), 2.8-2.9 (m, 4H), 2.6-2.8 (m, 1H), 2.3-2.5 (m, 6H, overlap DMSO), 1.92 (br dd, 1H, J=5.9, 9.6 Hz), 1.7-1.8 (m, 2H), 1.6-1.7 (m, 3H), 1.3-1.5 (m, 1H), 1.0-1.2 (m, 4H), 0.5-0.7 (m, 4H).

### Biological Assay

Test compounds were evaluated for their potential to inhibit Human Recombinant Matriptase 2 (in-house and commercial protein from Enzo Life Sciences—cat log—ALX-201-752-1) using fluorescence-based assay. The concentration of Recombinant Matriptase 2 and the substrate used in the assay were: 7 nM (commercial) and 15 nM (In-house Matriptase 2) and 100 μM (Boc-Gln-Ala-Arg-7-amido-4-methyl coumarin hydrobromide—Cat log: B4153-Sigma and I-1550 from Bachem) respectively. The assay buffer used comprised 50 mM TRIS pH 7.5, 150 mM NaCl, 0.01% gelatin (G1393 Sigma). The assay was performed by using 384 well black flat bottom plate from Grenier (Cat log: 781076) at 25° C. The enzyme and the test compound were preincubated for 30 mins, and the plate was read at wavelength Ex: 360/Em:480 nm60 mins after substrate addition. The final assay volume of was 20 μl. Stock solution of compounds were initially prepared in DMSO, which were appropriately diluted for screening to determine IC50 determination (final DMSO conc in the assay was 1%). All the measurements were carried out using the Spectramax M5, Molecular devices. The test compounds were screened at 1 and 10 μM concentrations and IC50 was determined for the interested compounds.

Ki is the binding affinity of an inhibitor. Mat-2 Ki as described herein is the binding affinity of an inhibitor such as a compound as described and provided herein to Mat-2. Mat-2 Ki values are derived from the measured IC₅₀. To determine IC₅₀ values, dose-response curves were generated by plotting percentage inhibition as a function of inhibitor concentration and the data was fitted to a sigmoidal non-linear regression equation (variable slope) using Graph Pad Prism software V7. Mat-2 Ki values as described herein were derived from the IC₅₀ using the Cheng-Prusoff equation, which is understood and appreciated by one the skill in the art. The Mat-2 Ki values of tested compounds as described herein are shown in Table A. Accordingly, the compounds can be used to inhibit matriptase 2 and treat diseases and conditions associated with matriptase 2, such as those described herein.

While we have described a number of embodiments of this disclosure, it is apparent that our basic examples may be altered to provide other embodiments that utilize the compounds and methods of the present disclosure. Therefore, it will be appreciated that the scope of the present disclosure is to be defined by the appended claims rather than by the specific embodiments that have been represented by way of example.

1. A compound having a formula of Formula I, or a pharmaceutically acceptable salt thereof: 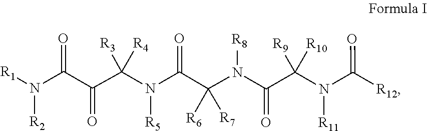
wherein:
R₁, R₂, R₃, R₄, R₅, R₆, R₇, R₈, R₉, R₁₀, R₁₁, and R₁₂ are each, independently, H, D, halo, alkyl, alkoxy, alkenyl, alkynyl, haloalkyl, haloalkoxy, aryl, cycloalkyl, heteroaryl, heterocycloalkyl, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}C(═NOH)NR^{a}R^{a}, NR^{a}C(═NCN)NR^{a}R^{a}, NR^{a}S(O)R^{a}, NR^{a}S(O)₂R^{a}, NR^{a}S(O)₂NR^{a}R^{a}, S(O)R^{a}, S(O)R^{a}NR^{a}R^{a}, S(O)NR^{a}R^{a}, S(O)₂R^{a}, SF₅, P(O)R^{a}R^{a}, P(O)(OR^{a})(OR^{a}), B(OR^{a})₂ and S(O)₂NR^{a}R^{a};
wherein when R₁, R₂, R₃, R₄, R₅, R₆, R₇, R₈, R₉, R₁₀, R₁₁, or R₁₂ is alkyl, alkoxy, alkenyl, alkynyl, haloalkyl, haloalkoxy, aryl, cycloalkyl, heteroaryl, heterocycloalkyl, then R₁, R₂, R₃, R₄, R₅, R₆, R₇, R₈, R₉, R₁₀, R₁₁, or R₁₂ is optionally substituted with at least one R^{b} substituent;
or one or more selected from the group consisting of R₁ and R₂, R₁ and R₄, R₃ and R₄, R₆ and R₇, R₇ and R₈, and R₉ and R₁₀, together with the atom(s) to which they are attached, form a 3-, 4-, 5-, 6-, or 7-membered cycloalkyl or heterocycloalkyl ring optionally substituted with 1, 2, 3, 4 or 5 independently selected R^{b} substituents;
each R^{a} is independently selected from H, D, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl;
wherein when R^{a} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a} is optionally substituted with 1, 2, 3, 4, or 5 independently selected R^{d} substituents;
each R^{b} substituent is independently selected from a bond, H, D, halo, oxo, C₁₋₁₀ alkyl, C₁₋₆ alkoxy, C₁₋₄ haloalkyl, C₁₋₄haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)(═NR^{c})R^{c}, NR^{c}S(O)₂R^{c}, NR^{c}S(O)₂NR^{c}R^{c}, S(O)R^{c}, S(O)NR^{c}R^{c}, S(O)₂R^{c} or S(O)₂NR^{c}R^{c};
wherein when R^{b} is C₁₋₁₀ alkyl, C₁₋₆ alkoxy, C₁₋₄haloalkyl, C₁₋₄haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{b} is optionally substituted with 1, 2, or 3 independently selected R^{d} substituents;
each R^{c} is independently selected from H, D, OH, C₁₋₆ alkyl, C₁₋₆ alkoxy, C₁₋₄ haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl;
wherein when R^{c} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents;
each R^{f} is independently selected from halogen, C₁₋₁₀ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R_{g}, C(O)OR^{g}, OC(O)R^{g}, OC(O)NR^{g}R_{g}, NHR^{g}, NR^{g}R_{g}, NR^{g}C(O)R^{g}, NR^{g}C(O)NR^{g}R_{g}, NR^{g}C(O)OR^{g}, C(═NR^{g})NR^{g}R^{g}, NR^{g}C(═NR^{g})NR^{g}R^{g}, NR^{g}C(═NOH)NR^{g}R^{g}, NR^{g}C(═NCN)NR^{g}R^{g}, SF₅, P(O)R^{g}R_{g}, P(O)(OR^{g})(OR^{g}), S(O)R^{g}, S(O)NR^{g}R_{g}, S(O)₂R₉, NR^{g}S(O)₂R₉, NR^{g}S(O)₂NR^{g}R_{g}, and S(O)₂NR^{g}R_{g};
wherein when R^{f} is C₁₋₄alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents;
each Rⁿ is independently selected from C₁₋₁₀ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, SF₅, P(O)R^{e}R^{e}, P(O)(OR^{e})(OR^{e}), S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e},
wherein when R^{d} is C₁₋₆alkyl, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents;
each R^{e} is independently selected from H, D, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{e} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents;
each R^{g} is independently selected from H, D, C₁₋₆ alkyl, C₁₋₄ haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents;
each R^{P} is independently selected from C₁₋₁₀ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r};
each R^{r} is independently selected from H, D, C₁₋₁₀ alkyl, C₃₋₆cycloalkyl, C₆₋₁₀ aryl, 5 or 6-membered heteroaryl, C₁₋₄ haloalkyl, C₂₋₄alkenyl, and C₂₋₄ alkynyl,
wherein when R^{r} is C₁₋₁₀ alkyl, C₃₋₆ cycloalkyl, C₆₋₁₀ aryl, 5 or 6-membered heteroaryl, C₂₋₄ alkenyl, and C₂₋₄ alkynyl, then R^{r} is optionally substituted with 1, 2 or 3 independently selected R^{q} substituents;
each R^{q} is independently selected from D, OH, CN, —COOH, NH₂, halo, C₁₋₆alkyl, C₁₋₆haloalkyl, C₁₋₆ alkoxy, C₁₋₆haloalkoxy, C₁₋₄ alkylthio, phenyl, 5-6 membered heteroaryl, C₃₋₆ cycloalkyl, 4-6 membered heterocycloalkyl, —CONHR^{t}, —NHC(O)R^{t}, —OC(O)R^{t}, —C(O)OR^{t}, —C(O)R^{t}, —SO₂R^{t}, —NHSO₂R^{t}, —SO₂NHR^{t} and NR^{t}R^{t},
wherein when R^{q} is C₁₋₆ alkyl, phenyl, 4-6 membered heterocycloalkyl or 5-6 membered heteroaryl, then R^{q} is optionally substituted with OH, CN, —COOH, NH₂, C₁₋₆ alkoxy, C₃₋₆cycloalkyl or 4-6 membered heterocycloalkyl; and
each R^{t} is independently C₁₋₆ alkyl.

2. The compound of claim 1, where R₁, R₂, R₃, R₄, R₅, R₆, R₇, R₈, R₉, R₁₀, R₁₁, and R₁₂ are each, independently, H, D, OH, NH₂, NHC(═NH)NH₂, NHR^{a}, optionally substituted C₁-C₆alkyl, optionally substituted C₁-C₆ hydroxyalkyl, optionally substituted C₁-C₆ alkoxy, optionally substituted cycloalkyl, or optionally substituted cycloheteroalkyl, wherein R^{a} is as defined in claim 1, optionally
i. wherein R₅ is H;
ii. wherein R₈ is H;
iii. wherein R₁₁ is H;
iv. wherein R₃ is H;
v. wherein R₆ is H; or
vi. wherein R₉ is H.

3. The compound of claim 1, or a pharmaceutically acceptable salt thereof, wherein
the compound has a formula of 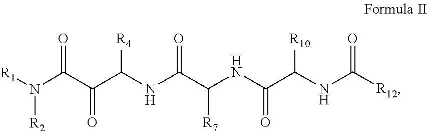
optionally, wherein the compound has a formula of

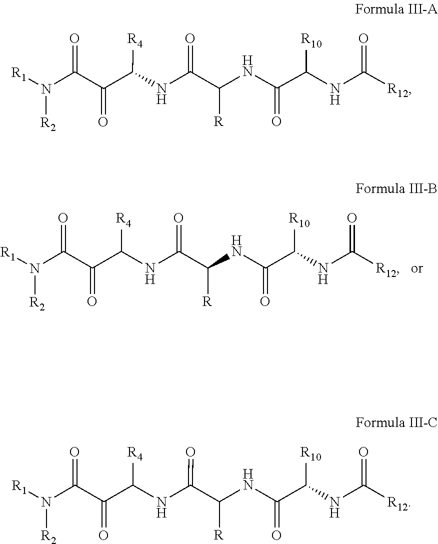

4. The compound of claim 1, or a pharmaceutically acceptable salt thereof, wherein the compound has a formula of

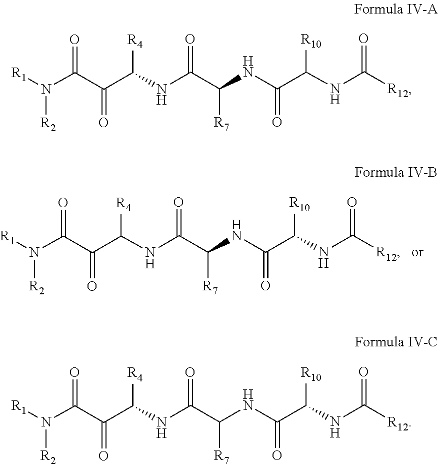

5. The compound of claim 1, or a pharmaceutically acceptable salt thereof, wherein the compound has a formula of 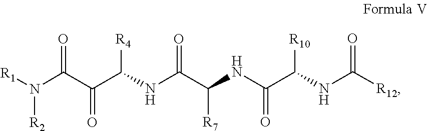
optionally
i. wherein R₁ is optionally substituted C₁-C₆ alkyl;
ii. wherein R₁ is Me;
iii. wherein R₁ is H;
iv. wherein R₂ is Me;
v. wherein R₂ is Et; or
vi. wherein R₂ is 3-pyridyl.

6. The compound of claim 1, or a pharmaceutically acceptable salt thereof, wherein R₂ is H, Me, Et, t-Bu, —CH₂CN, -3-pyridyl, —CH₂C(O)OR₁₇, 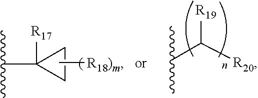
wherein: R₁₇, R₁₈, R₁₉, and R₂₀ are each, independently, H, OH, NH₂, optionally substituted C₁-C₆ alkyl, optionally substituted C₁-C₆ hydroxyalkyl, optionally substituted C₁-C₆ alkoxy, optionally substituted cycloalkyl, optionally substituted pyridyl, optionally substituted heteroaryl, optionally substituted aryl, or optionally substituted cycloheteroalkyl; m is 0-4; and n is 0-10, optionally
i. wherein R₁₇ is Me,
ii. wherein R₁₈ is halo,
iii. wherein R₁₈ is F,
iv. wherein R₁₉ is H,
v. wherein m is 1, or
vi. wherein n is 1;
vii. wherein R₂₀ is 3-pyridyl, —C(O)OR₂₁, or CN, wherein R₂₁. is optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl, optionally wherein R₂₁. is optionally substituted C₁-C₆ alkyl or optionally wherein R₂₁ is ethyl.

7. The compound of claim 1, or a pharmaceutically acceptable salt thereof, wherein R₂ and R₄ together with the atom to which they are attached, form a 3-, 4-, 5-, 6-, or 7-membered cycloalkyl or heterocycloalkyl ring optionally substituted with 1, 2, 3, 4 or 5 independently selected R^{b} substituents, wherein R^{b} is as defined in claim 1.

8. The compound of claim 7, or a pharmaceutically acceptable salt thereof, wherein R₁ and R₄ together with the atom to which they are attached, form a 7-membered heterocycloalkyl ring optionally substituted with 1, 2, 3, 4 or 5 independently selected R^{b} substituents, wherein R^{b} is as defined in claim 1.

9. The compound of claim 1, or a pharmaceutically acceptable salt thereof, wherein R₄ is 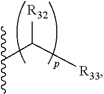
wherein R₃₂ and R₃₃ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; and p is 0-10, optionally
i. wherein R₃₂ is H;
ii. wherein p is 1;
iii. wherein p is 2; or
iv. wherein p is 3.

10. The compound of claim 9, or a pharmaceutically acceptable salt thereof, wherein R₃₃ is H, 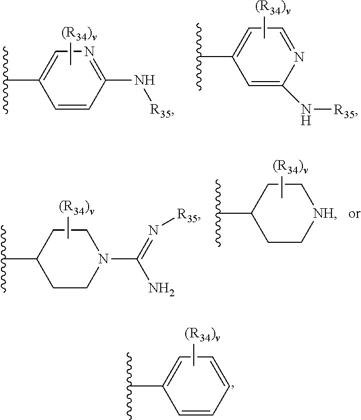
wherein R₃₄ and R₃₅ are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl and v is 0-5, optionally
i. wherein R₃₄ is H.
ii. wherein v is 0;
iii. wherein v is 1;
iv. wherein R₃₅ is H;
v. wherein R₃₃ is 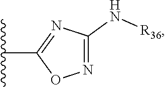
wherein R₃₆ is H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or
vi. wherein R₃₆ is H.

11. The compound of claim 9, or a pharmaceutically acceptable salt thereof, wherein R₃₃ is 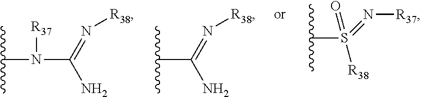
wherein R₃₇ and R₃₈ are each, independently, H, D, NH₂, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl, optionally.
i. wherein R₃₇ is H;
ii. wherein R₃₈ is H or NH₂;
iii. wherein R₃₈ is optionally substituted C₁-C₆ alkyl;
vi. wherein R₃₈ is Me;
v. wherein R₃₈ is optionally substituted C₁-C₆ alkoxy or NH₂; or
v. wherein R₃₈ is MeO.

12. The compound of claim 1, or a pharmaceutically acceptable salt thereof, wherein R₇ is 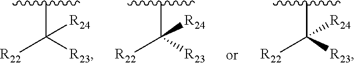
wherein R₂₂, R₂₃, and R₂₄, are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl; or R₂₂ and R₂₃ together with the atom to which they are attached, form an optionally substituted cycloalkyl or optionally substituted cycloheteroalkyl, optionally
i. wherein R₂₂ is Me; wherein R₂₃ is OH, OMe, Me, or Et;
ii. wherein R₂₂ and R₂₃ together with the atom to which they are attached, form an optionally substituted cyclobutane;
iii. wherein R₂₂ and R₂₃ together with the atom to which they are attached, form an optionally substituted cyclopetane or cyclohexane;
iv. wherein R₂₄ is Me; or
v. wherein R₂₄ is H.

13. The compound of claim 1, or a pharmaceutically acceptable salt thereof, wherein R₇ and R₈ are together with the atoms to which they are attached, form a 3-, 4-, 5-, 6-, or 7-membered cycloalkyl or heterocycloalkyl ring optionally substituted with 1, 2, 3, 4 or 5 independently selected R^{b} substituents, optionally wherein R₇ and R₈ together are 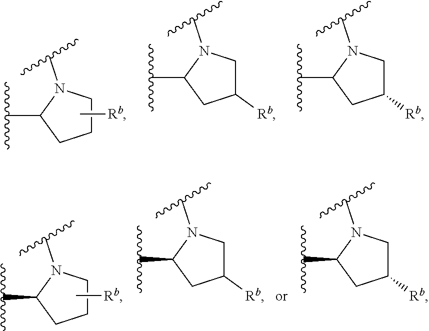
optionally wherein R^{b} is H or optionally wherein R^{b} is OH or CN.

14. The compound of claim 1, or a pharmaceutically acceptable salt thereof, R₁₀ is 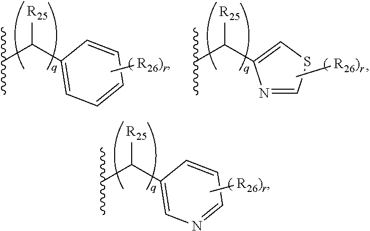
wherein:
R₂₅ and R₂₆, are each, independently, H, D, optionally substituted C₁-C₆ alkyl, optionally substituted C₁-C₆ hydroxyalkyl, optionally substituted C₁-C₆ alkoxy, optionally substituted cycloalkyl, or optionally substituted cycloheteroalkyl;
q is 0-10; and
r is 0-5, optionally.
i. wherein R₂₅ is H;
ii. wherein q is 1;
iii. wherein q is 2, 3, or 4;
iv. wherein r is 0;
v. wherein r is 1;
vi. wherein R₂₆ is Me; or
vi. wherein R₂₆ is halo.

15. The compound of claim 1, or a pharmaceutically acceptable salt thereof, wherein R₁₂ is 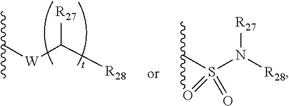
wherein:
W is O, S, NR₂₉, S(O)₂NR₂₉, S(O)₂, S(O)₂NH, or CR₃₀R₃₁;
R₂₇, R₂₈, R₂₉, R₃₀, and R₃₁ are each, independently, H, D, optionally substituted C₁-C₆ alkyl, optionally substituted C₁-C₆ hydroxyalkyl, optionally substituted C₁-C₆ alkoxy, optionally substituted cycloalkyl, optionally substituted aryl, optionally substituted heteroaryl, or optionally substituted cycloheteroalkyl; and t is 0-10, optionally
i. wherein W is O;
ii. wherein W is CR₃₀R₃₁; or
iii. wherein W is CH₂.

16. The compound of claim 15, or a pharmaceutically acceptable salt thereof, wherein W is NR₂₉, 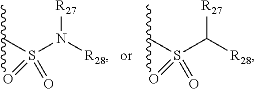
optionally
i. wherein R₂₉ is H;
ii. wherein R₂₇ is H;
iii. wherein t is 1-4;
iv. wherein R₂₈ is methyl, ethyl, cyclopropyl, or phenyl;
v. wherein R₂₈ is optionally substituted C₁-C₆ alkyl;
vi. wherein R₂₈ is Me;
vi. wherein R₂₈ is Et; or
v. wherein R₂₈ is isopropyl.

17. (canceled)

18. A compound having a formula of: 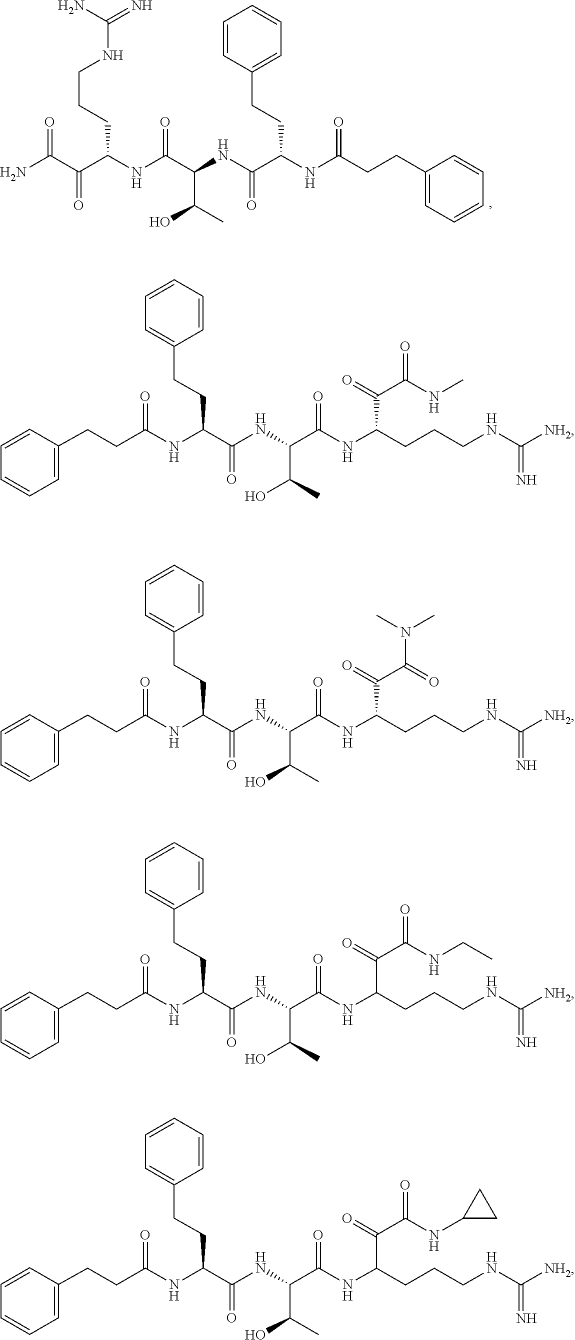 [SMILES: C[C@@H](O)[C@H](NC(=O)[C@H](CCc1ccccc1)NC(=O)CCc1ccccc1)C(=O)N[C@@H](CCCNC(=N)N)C(=O)C(N)=O ; CNC(=O)C(=O)[C@H](CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)N[C@@H](CCCNC(=N)N)C(=O)C(=O)N(C)C ; CCNC(=O)C(=O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NC1CC1] 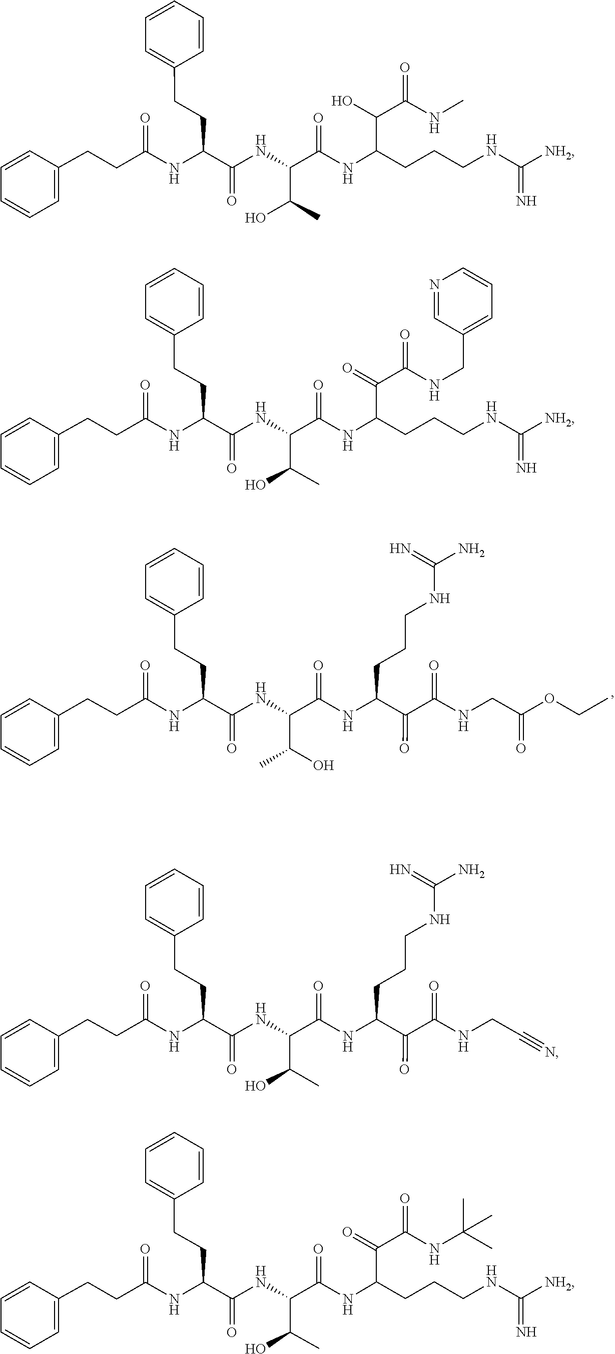 [SMILES: CNC(=O)C(O)C(CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)NC(CCCNC(=N)N)C(=O)C(=O)NCc1cccnc1 ; CCOC(=O)CNC(=O)C(=O)[C@H](CCCNC(=N)N)NC(=O)[C@@H](NC(=O)[C@H](CCc1ccccc1)NC(=O)CCc1ccccc1)[C@@H](C)O ; C[C@@H](O)[C@H](NC(=O)[C@H](CCc1ccccc1)NC(=O)CCc1ccccc1)C(=O)N[C@@H](CCCNC(=N)N)C(=O)C(=O)NCC#N ; C[C@@H](O)[C@H](NC(=O)[C@H](CCc1ccccc1)NC(=O)CCc1ccccc1)C(=O)NC(CCCNC(=N)N)C(=O)C(=O)NC(C)(C)C] 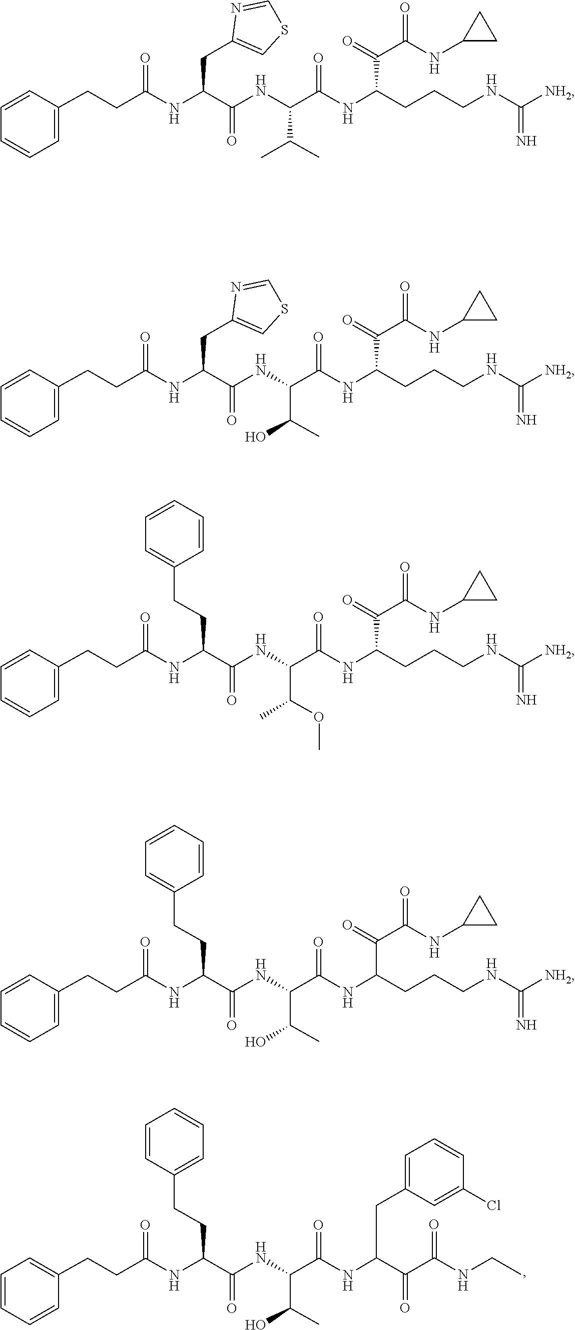 [SMILES: CC(C)[C@H](NC(=O)[C@H](Cc1cscn1)NC(=O)CCc1ccccc1)C(=O)N[C@@H](CCCNC(=N)N)C(=O)C(=O)NC1CC1 ; C[C@@H](O)[C@H](NC(=O)[C@H](Cc1cscn1)NC(=O)CCc1ccccc1)C(=O)N[C@@H](CCCNC(=N)N)C(=O)C(=O)NC1CC1 ; CO[C@H](C)[C@H](NC(=O)[C@H](CCc1ccccc1)NC(=O)CCc1ccccc1)C(=O)N[C@@H](CCCNC(=N)N)C(=O)C(=O)NC1CC1 ; C[C@H](O)[C@H](NC(=O)[C@H](CCc1ccccc1)NC(=O)CCc1ccccc1)C(=O)NC(CCCNC(=N)N)C(=O)C(=O)NC1CC1 ; CCNC(=O)C(=O)C(Cc1cccc(Cl)c1)NC(=O)[C@@H](NC(=O)[C@H](CCc1ccccc1)NC(=O)CCc1ccccc1)[C@@H](C)O] 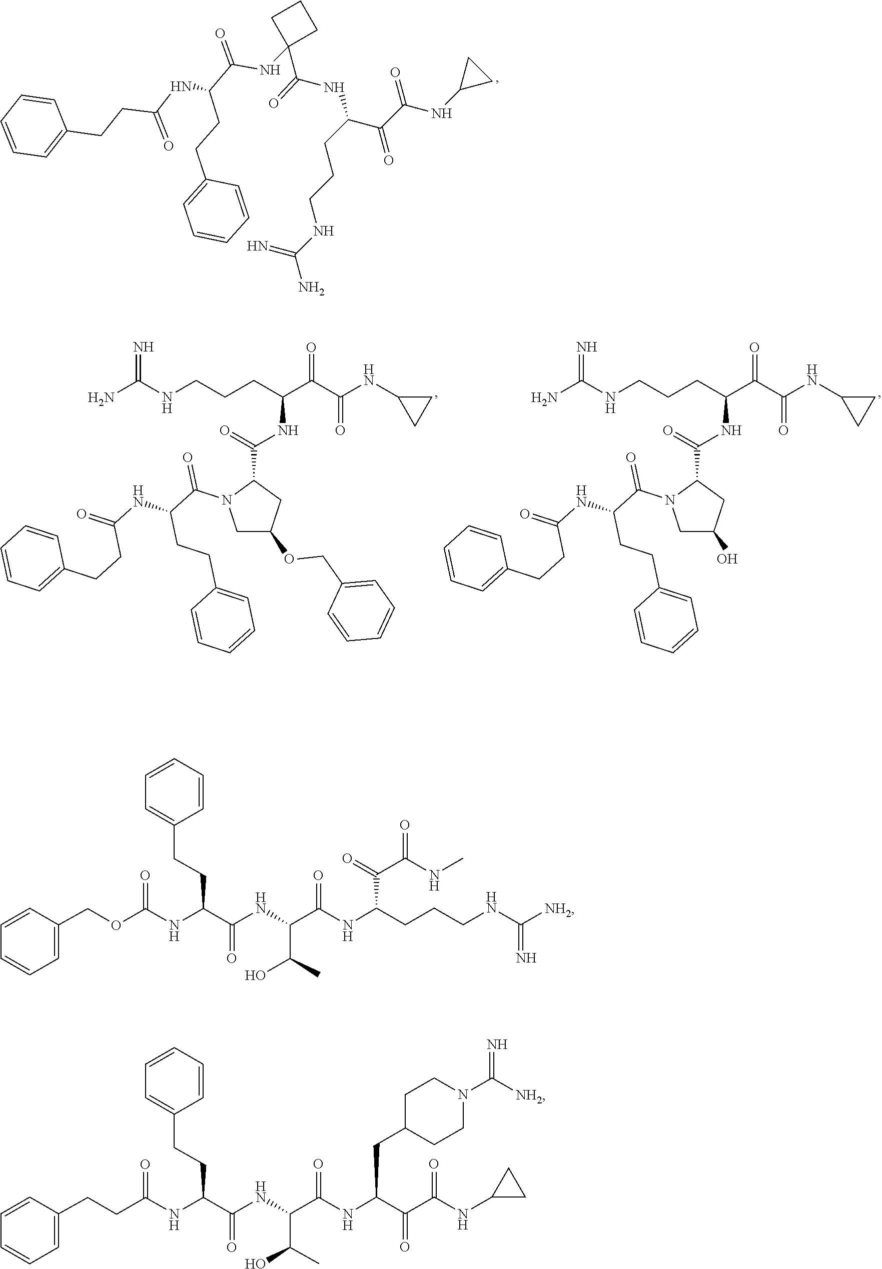 [SMILES: N=C(N)NCCC[C@H](NC(=O)C1(NC(=O)[C@H](CCc2ccccc2)NC(=O)CCc2ccccc2)CCC1)C(=O)C(=O)NC1CC1 ; N=C(N)NCCC[C@H](NC(=O)[C@@H]1C[C@@H](OCc2ccccc2)CN1C(=O)[C@H](CCc1ccccc1)NC(=O)CCc1ccccc1)C(=O)C(=O)NC1CC1 ; N=C(N)NCCC[C@H](NC(=O)[C@@H]1C[C@@H](O)CN1C(=O)[C@H](CCc1ccccc1)NC(=O)CCc1ccccc1)C(=O)C(=O)NC1CC1 ; CNC(=O)C(=O)[C@H](CCCNC(=N)N)NC(=O)[C@@H](NC(=O)[C@H](CCc1ccccc1)NC(=O)OCc1ccccc1)[C@@H](C)O ; C[C@@H](O)[C@H](NC(=O)[C@H](CCc1ccccc1)NC(=O)CCc1ccccc1)C(=O)N[C@@H](CC1CCN(C(=N)N)CC1)C(=O)C(=O)NC1CC1] 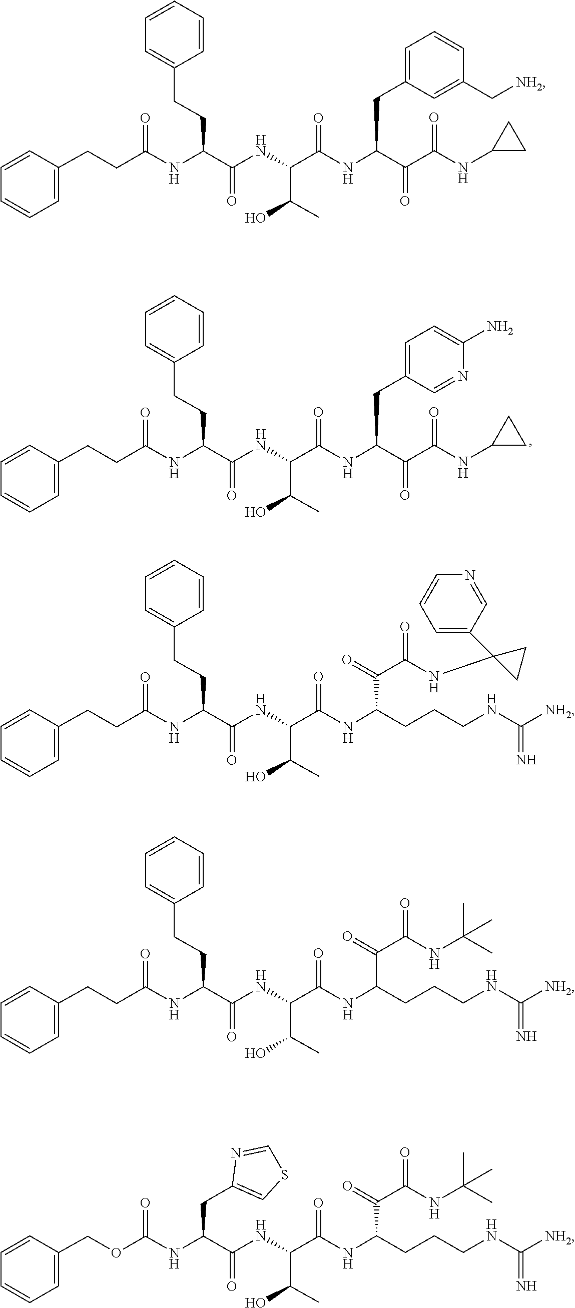 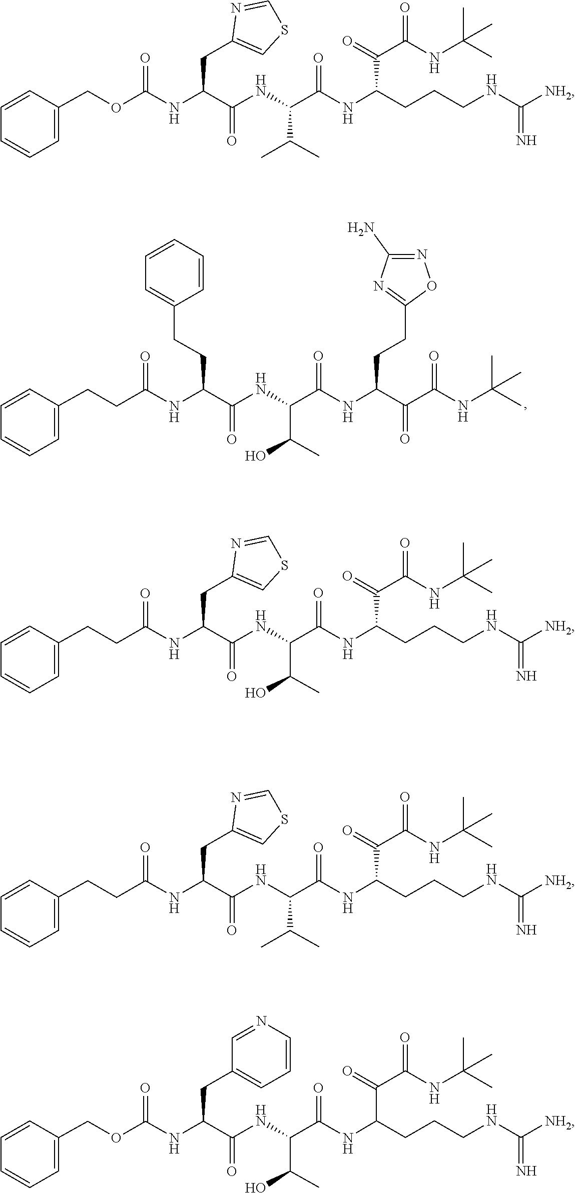 [SMILES: CC(C)[C@H](NC(=O)[C@H](Cc1cscn1)NC(=O)OCc1ccccc1)C(=O)N[C@@H](CCCNC(=N)N)C(=O)C(=O)NC(C)(C)C ; C[C@@H](O)[C@H](NC(=O)[C@H](CCc1ccccc1)NC(=O)CCc1ccccc1)C(=O)N[C@@H](CCc1nc(N)no1)C(=O)C(=O)NC(C)(C)C ; C[C@@H](O)[C@H](NC(=O)[C@H](Cc1cscn1)NC(=O)CCc1ccccc1)C(=O)N[C@@H](CCCNC(=N)N)C(=O)C(=O)NC(C)(C)C ; CC(C)[C@H](NC(=O)[C@H](Cc1cscn1)NC(=O)CCc1ccccc1)C(=O)N[C@@H](CCCNC(=N)N)C(=O)C(=O)NC(C)(C)C ; C[C@@H](O)[C@H](NC(=O)[C@H](Cc1cccnc1)NC(=O)OCc1ccccc1)C(=O)NC(CCCNC(=N)N)C(=O)C(=O)NC(C)(C)C] 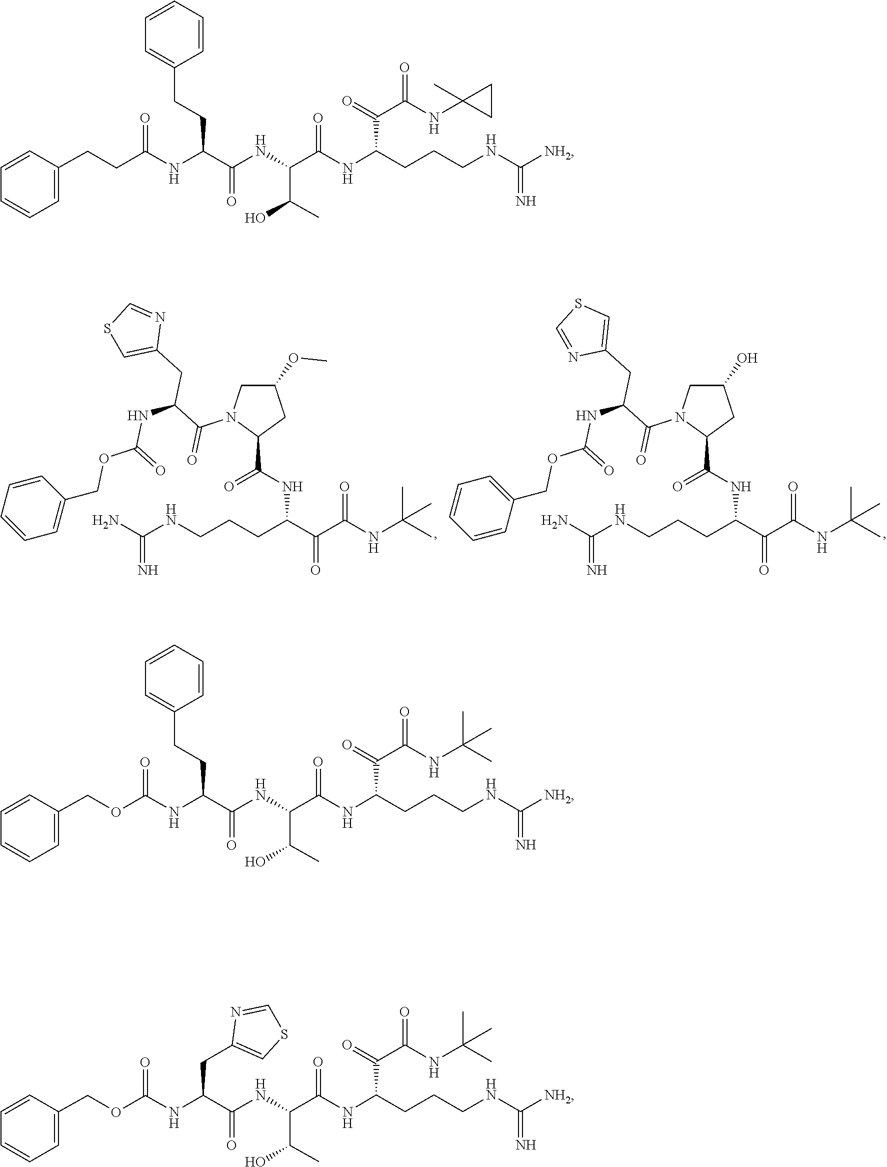 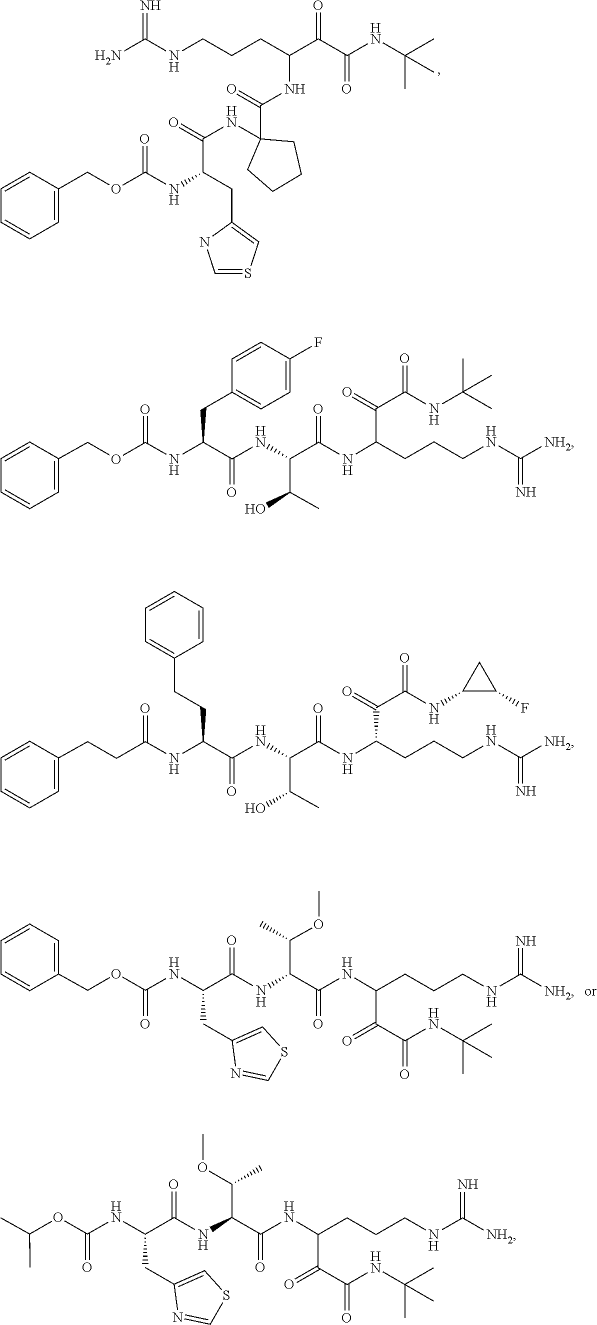 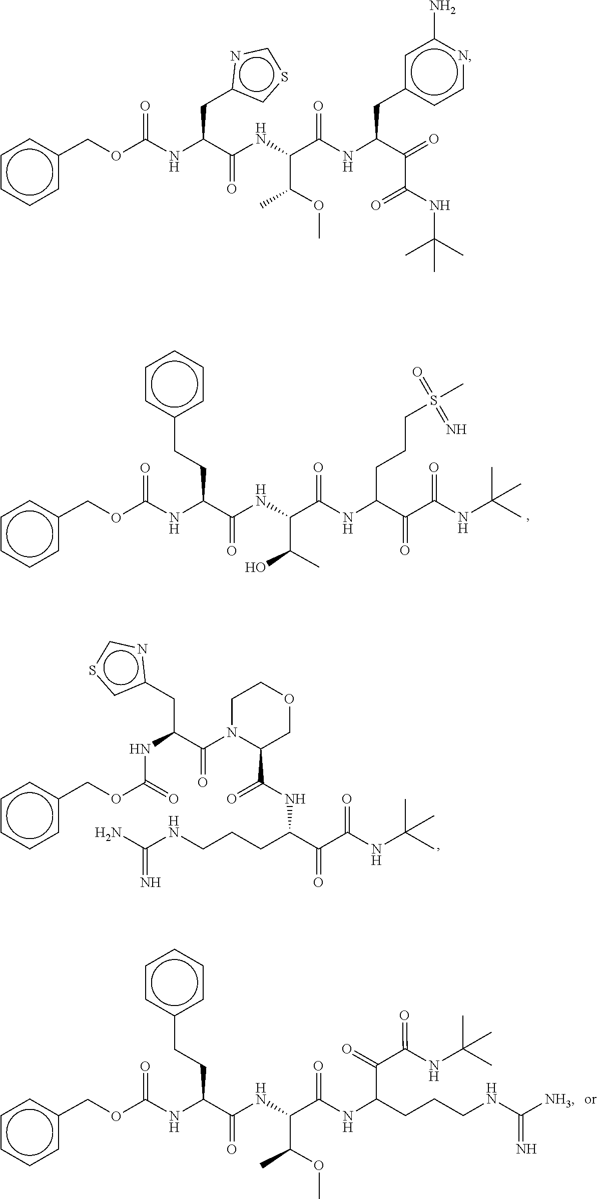 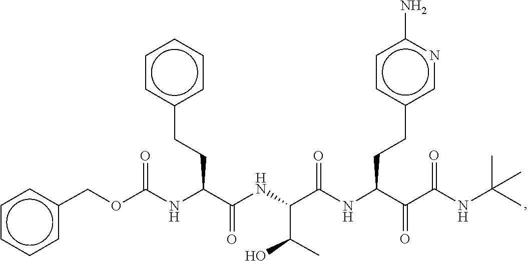
or a pharmaceutically acceptable salt thereof.

19. A pharmaceutical composition comprising a compound of claim 1, or a pharmaceutically acceptable salt thereof.

20. A pharmaceutical composition comprising the compound of claim 1, or a pharmaceutically acceptable salt thereof, and a pharmaceutically acceptable carrier, adjuvant, or vehicle.

21. A method for treating a low hepcidin disorder, disease, and/or condition in a subject, comprising administering to the subject the compound of claim 1, or a pharmaceutically acceptable salt thereof, or a pharmaceutical composition thereof.

22. A method for increasing hepcidin production by the liver in a subject, comprising administering to the subject the compound of claim 1, or a pharmaceutically acceptable salt thereof, or a pharmaceutical composition thereof.

23. A method for treating an iron overload disorder, disease, and/or condition in a subject, comprising administering to the subject the compound of claim 1, or a pharmaceutically acceptable salt thereof, or a pharmaceutical composition thereof.

24. The method of claim 23, wherein the iron overload disorder, disease, and/or condition is selected from the group consisting of hemochromatosis Type 1, 2a, 2b and 3 (hemochromatosis, Hfe hemochromatosis (Type 1), juvenile hemochromatosis (types 2a and 2b)), hepcidin deficiency, transfusional iron overload, African iron overload, and iron overload cardiomyopathy.

25. A method for treating an iron loading anemia in a subject, comprising administering to the subject the compound of claim 1, or a pharmaceutically acceptable salt thereof, or a pharmaceutical composition thereof.

26. The method of claim 25, wherein the iron loading anemia is selected from the group consisting of beta thalassemia, HbE/thalassemia (thalassemia major, thalassemia intermedia, thalassemia minor, non-transfusion dependent thalassemia, transfusion-dependent thalassemia), alpha thalassemia, congenital dyserythropoietic anemias (Type I and Type II), pyruvate kinase deficiency, myelodysplasia, myelodysplastic syndrome, and RARS SF3B1 associated MDS.

27. A method for treating a hematological disease, disorder, and/or condition in a subject, comprising administering to the subject the compound of claim 1, or a pharmaceutically acceptable salt thereof, or a pharmaceutical composition thereof.

28. The method of claim 27, wherein the hematological disease, disorder, and/or condition is selected from the group consisting of sickle cell disease, sickle cell anemia, polycythemia vera, sideroblastic anemia, and bone marrow transplantation.

29. A method for treating a liver disease in a subject, comprising administering to the subject the compound of claim 1, or a pharmaceutically acceptable salt thereof, or a pharmaceutical composition thereof.

30. The method of claim 29, wherein the liver disease is selected from the group consisting of Hepatitis B, Hepatitis C, alcoholic liver disease, cirrhosis of the liver, hepatocellular carcinoma, and non-alcoholic steatohepatitis (NASH).

31. A method of treating a metabolic disease in a subject, comprising administering to the subject the compound of claim 1, or a pharmaceutically acceptable salt thereof, or a pharmaceutical composition thereof.

32. The method of claim 31, wherein the metabolic disease is selected from the group consisting of metabolic syndrome, insulin resistance, Type II diabetes, porphyria, porphyria cutanea tarda, Wilson's Disease, and acute iron overdose.

33. A method for treating a neurodegenerative disorder in a subject, comprising administering to the subject the compound of claim 1, or a pharmaceutically acceptable salt thereof, or a pharmaceutical composition thereof.

34. A method for treating an infectious disease in a subject, comprising administering to the subject the compound of claim 1, or a pharmaceutically acceptable salt thereof, or a pharmaceutical composition thereof.

35. The method of claim 34, wherein the infectious disease is a siderophilic infection.

36. The method of claim 21, wherein the subject is a subject in need thereof.

37. A method of inhibiting matriptase 2, or a mutant thereof, in a biological sample, comprising contacting the sample with the compound of claim 1, or a pharmaceutically acceptable salt thereof, or a pharmaceutical composition thereof.

38. The method of claim 21, wherein the compound, or a pharmaceutically acceptable salt thereof, or a pharmaceutical composition thereof is administered in an effective amount.
